(19) United States
(12) Patent Application Publication
(10) Pub. No.: US 2024/0081146 A1
(43) Pub. Date: Mar. 7, 2024

(54) ORGANIC LIGHT EMITTING DIODE AND ORGANIC LIGHT EMITTING DEVICE HAVING THEREOF

(71) Applicant: LG Display Co., Ltd., Seoul (KR)

(72) Inventors: Yu-Jeong LEE, Paju-si (KR); Jung-Keun KIM, Paju-si (KR); Mi-Young HAN, Paju-si (KR); Min-Hyeong HWANG, Paju-si (KR)

(21) Appl. No.: 18/218,597

(22) Filed: Jul. 6, 2023

(30) Foreign Application Priority Data
Aug. 16, 2022 (KR) 10-2022-0102086

(51) Int. Cl. H10K 85/60; C07D 235/08; C07D 471/10; C09K 11/06

(52) U.S. Cl. CPC H10K 85/6572; C07D 235/08; C07D 471/10; C09K 11/06; H10K 85/615; H10K 85/626; H10K 50/166

(57) ABSTRACT

An organic light emitting diode (OLED) includes an emissive layer with at least one emitting part includes at least one emitting material layer, a first electron transport layer and a second electron transport layer disposed sequentially between two facing electrodes, wherein the first electron transport layer includes a first electron transporting material of a benzimidazole-based compound substituted with at least one spiro-structured fluorenyl group and the second electron transport layer includes a second electron transporting material of a benzimidazole-based compound substituted with at least one anthracenyl group. The first electron transport including the first electron transport material with excellent thermal stability is disposed adjacently to the emitting material layer so that the OLED can maintain good luminescent intensity in an environment of high temperature and implement beneficial luminous properties.

### CROSS-REFERENCE TO RELATED APPLICATIONS

This application claims the benefit of and the priority of Korean Patent Application No. 10-2022-0102086, filed in the Republic of Korea on Aug. 16, 2022, which is expressly incorporated hereby in its entirety into the present application.

### BACKGROUND

### Technical Field

The present disclosure relates to an organic light emitting diode, and more particularly to, an organic light emitting diode that may have improved luminous efficiency and luminous lifespan and an organic light emitting device including thereof.

### Discussion of the Related Art

A flat display device including an organic light emitting diode (OLED) has attracted attention as a display device that can replace a liquid crystal display device (LCD). The OLED can be formed as a thin organic film less than 2000 Å and the electrode configurations can implement unidirectional or bidirectional images. Also, the OLED can be formed even on a flexible transparent substrate such as a plastic substrate so that a flexible or a foldable display device can be realized with ease using the OLED. In addition, the OLED can be driven at a lower voltage and the OLED has advantageous high color purity compared to the LCD.

Since fluorescent material uses only singlet excitons in the luminous process, the related art fluorescent material shows low luminous efficiency. On the contrary, phosphorescent material can show high luminous efficiency since it uses triplet exciton as well as singlet excitons in the luminous process. However, examples of phosphorescent material include metal complexes, which have a short luminous lifespan for commercial use.

### SUMMARY

Accordingly, embodiments of the present disclosure are directed to an organic light emitting diode and an organic light emitting device that substantially obviate one or more of the problems due to the limitations and disadvantages of the related art.

An aspect of the present disclosure is to provide an organic light emitting diode that may have enhanced thermal stability and an organic light emitting device including the organic light emitting diode. Another aspect of the present disclosure is to provide an organic light emitting diode that may have improved luminous properties and an organic light emitting device including the organic light emitting diode.

Additional features and aspects will be set forth in the description that follows, and in part will be apparent from the description, or may be learned by practice of the disclosed concepts provided herein. Other features and aspects of the disclosed concept may be realized and attained by the structure particularly pointed out in the written description, or derivable therefrom, and the claims hereof as well as the appended drawings.

To achieve these and other aspects of the inventive concepts, as embodied and broadly described, in one aspect, the present disclosure provides an organic light emitting diode includes a first electrode; a second electrode facing the first electrode; and an emissive layer disposed between the first and second electrode, and including at least one emitting part, wherein an emitting part located adjacently to the second electrode among the at least one emitting part includes at least one emitting material layer; and an electron transport layer disposed between the at least one emitting material layer and the second electrode, wherein the electron transport layer includes a first electron transport layer disposed between the at least one emitting material layer and the second electrode; and a second electron transport layer disposed between the first electron transport layer and the second electrode, wherein the first electron transport layer includes a first electron transporting material represented by a structure of Chemical Formula 1, and wherein the second electron transport layer includes a second electron transporting material represented by a structure of Chemical Formula 4:

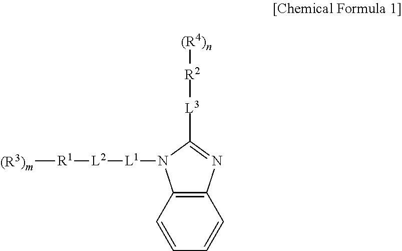

wherein, in the Chemical Formula 1,
each of R¹ and R² is independently an unsubstituted or substituted C₁-C₁₀ alkyl group, an unsubstitued or substituted C₆-C₃₀ aryl group or an unsubstituted or substituted C₃-C₃₀ hetero aryl group, and at least one of R¹ and R² is an unsubstituted or substituted spiro-fluorenyl group;
each of R³ and R⁴ is independently an unsubstituted or substituted C₁-C₁₀ alkyl group, an unsubstitued or substituted C₆-C₃₀ aryl group or an unsubstituted or substituted C₃-C₃₀ hetero aryl group, where each R³ is identical to or different form each other when m is an integer of 2 or more, and where each R⁴ is identical to or different from each other when n is an integer of 2 or more;
each of L¹, L² and L³ is independently a single bond, an unsubstituted or substituted C₆-C₃₀ arylene group or an unsubstituted or substituted C₃-C₃₀ hetero arylene group; and
each of m and n is independently an integer of 0 to 7,

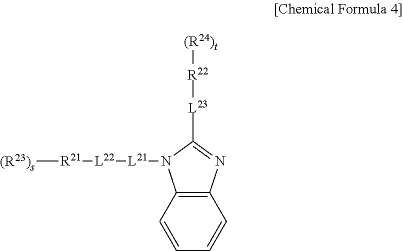

wherein, in the Chemical Formula 4,
each of R²¹ and R²² is independently an unsubstituted or substituted C₁-C₁₀ alkyl group, an unsubstituted or substituted C₆-C₃₀ aryl group or an unsubstitued or substituted C₃-C₃₀ hetero aryl group, and at least one of R²¹ and R²² is an unsubstitued or substituted anthracenyl group;
each of R²³ and R²⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R²³ is identical to or different form each other when s is an integer of 2 or more, and where each R²⁴ is identical to or different from each other when t is an integer of 2 or more;
each of L²¹, L²² and L²³ is independently a single bond, an unsubstituted or substituted C₆-C₃₀ arylene group or an unsubstituted or substituted C₃-C₃₀ hetero aryl group; and
each of s and t is independently an integer of 0 to 9.

The first electron transporting material can have a structure of Chemical Formula 2:

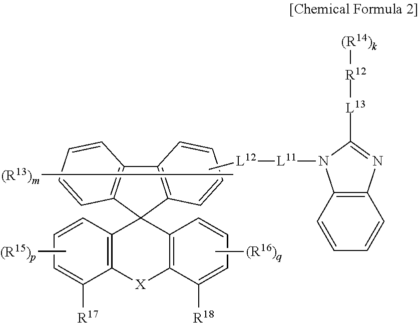

wherein, in the Chemical Formula 2,
R¹² is an unsubstituted or substituted C₁-C₁₀ alkyl group or an unsubstituted or substituted C₆-C₃₀ aryl group;
R¹³ is an or substituted C₁-C₁₀ alkyl group or an unsubstituted or substituted C₆-C₃₀ aryl group, and where each R¹³ is identical to or different from each other when m is an integer of 2 or more;
R¹⁴ is an unsubstituted or substituted C₁-C₁₀ alkyl group or an unsubstituted or substituted C₆-C₃₀ aryl group, and where each R¹⁴ is identical to or different from each other when k is an integer of 2 or more;
each of R¹⁵ and R¹⁶ is independently an C₁-C₁₀ alkyl group or an C₆-C₃₀ aryl group, where each R¹⁵ is identical to or different from each other when p is an integer of 2 or more, and where each R¹⁶ is identical to or different from each other when q is an integer of 2 or more;
each of R¹⁷ and R¹⁸ is independently hydrogen, an C₁-C₁₀ alkyl group or an C₆-C₃₀ aryl group, when X is a single bond, or one of R¹⁷ and R¹⁸, and X are further linked together to form an unsubstituted or substituted fused ring and the other of R¹⁷ and R¹⁸ is hydrogen, an C₁-C₁₀ alkyl group or an C₆-C₃₀ aryl group when X is nitrogen;
X is a single bond or nitrogen;
each of L¹¹, L¹² and L¹³ is independently a single bond or an unsubstituted or substituted C₆-C₃₀ arylene group;
m is a same as defined in Formula 1;
k is an integer of 0 to 3; and
each of p and q is an integer of 0 to 3.

The second electron transporting material can have a structure of Chemical Formula 5:

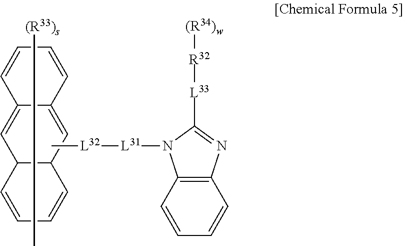

wherein, in the Chemical Formula 5,
R³² is an unsubstituted or substituted C₁-C₁₀ alkyl group or an unsubstituted or substituted C₆-C₃₀ aryl group;
R³³ is an unsubstituted or substituted C₁-C₁₀ alkyl group or an unsubstituted or substituted C₆-C₃₀ aryl group, and where each R³³ is identical to or different from each other when s is an integer of 2 or more;
R³⁴ is an unsubstituted or substituted C₆-C₃₀ aryl group, and where each R³⁴ is identical to or different from each other when w is an integer of 2 or more;
each of L³¹, L³² and L³³ is independently a single bond or an unsubstituted or substituted C₆-C₃₀ arylene group;
s is a same as defined in Formula 4; and
w is 0 or 1.

In one example embodiment, the emissive layer can have a single emitting part.

The at least emitting material layer can emit at least one of a red color, a green color, a blue color and a yellow-green color.

Alternatively, the emissive layer can include a first emitting part disposed between the first and second electrodes; a second emitting part disposed between the first emitting part and the second electrode; and a charge generation layer disposed between the first and second emitting parts, and wherein the second emitting part can include the at least one emitting material layer and the electron transport layer.

The at least one emitting material layer can include a first layer disposed between the charge generation layer and the second electrode; and a second layer disposed between the first layer and the second electrode. Optionally, the at least one emitting material layer can further include a third layer disposed between the first layer and the second layer.

In another example embodiment, the emissive layer can include a first emitting part disposed between the first and second electrodes; a second emitting part disposed between the first emitting part and the second electrode; a third emitting part disposed between the second emitting part and the second electrode; a first charge generation layer disposed between the first and second emitting parts; and a second charge generation layer disposed between the second and third emitting parts, and wherein the third emitting part can include the at least one emitting material layer and the electron transport layer.

In another aspect, the present disclosure provides an organic light emitting diode includes a first electrode; a second electrode facing the first electrode; and an emissive layer disposed between the first and second electrodes, and including at least one emitting part, wherein an emitting part located adjacently to the second electrode among the at least one emitting part includes a blue emitting material layer; and an electron transport layer disposed between the blue emitting material layer and the second electrode, wherein the electron transport layer includes a first electron transport layer and a second electron transport layer disposed sequentially between the at least one emitting material layer and the second electrode, wherein the first electron transport layer includes a first electron transporting material represented by the structure of Chemical Formula 1, and wherein the second electron transport layer includes a second electron transporting material represented by the structure of Chemical Formula 4.

The emissive layer can have a single emitting part.

Alternatively, the emissive layer can include a first emitting part disposed between the first and second electrodes; a second emitting part disposed between the first emitting part and the second electrode; a third emitting part disposed between the second emitting part and the second electrode; a first charge generation layer disposed between the first and second emitting parts; and a second charge generation layer disposed between the second and third emitting parts, and wherein the third emitting part can include the blue emitting material layer and the electron transport layer.

The second emitting part can include at least one emitting material layer, and wherein the at least one emitting material layer can include a first layer disposed between the first charge generation layer and the second charge generation layer; and a second layer disposed between the first layer and the second charge generation layer. One of the first and second layers can be a red emitting material layer and the other of the first and second layers can be a green emitting material layer.

Optionally, the at least one emitting material layer can further include a third layer of a yellow-green emitting material layer disposed between the first and second layers.

In yet another aspect, the present disclosure provides an organic light emitting device, for example, an organic light emitting display device or an organic light emitting illumination device, includes a substrate and the organic light emitting diode over the substrate.

The emitting part located adjacently to the second electrode includes a first electron transport layer including a first electron transporting material of a benzimidazole-based Compound substituted with at least one spiro-structured fluorenyl group and a second electron transport layer including a second electron transporting material of a benzimidazole-based Compound substituted with at least one anthracenyl group between the at least one emitting material layer and the second electrode.

The first electron transport layer including the first electron transporting material substituted with spiro-structured fluorenyl group with beneficial thermal resistance is disposed adjacently to the emitting material layer so that the organic light emitting diode improves its high temperature stability. The light intensity emitted from the emitting material is maintained stably so that the luminance and luminous lifespan of the organic light emitting diode and organic light emitting device can be improved.

The electron transport layer includes the benzimidazole-based electron transporting material that combines with electrons generated from the cathode and transfers the electrons to the emitting material. The energy level of the emitting material layer and the energy level of the electron transport material including the benzimidazole-based Compound are adjusted so that the electron transport layer can transfer electrons efficiently to the emitting material layer.

Electrons and holes are injected to the emitting material layer in balance so that the organic light emitting diode and organic light emitting device with beneficial luminous properties can be implemented.

It is to be understood that both the foregoing general description and the following detailed description are exemplary and explanatory, and are intended to provide further explanation of the inventive concepts as claimed.

### BRIEF DESCRIPTION OF THE DRAWINGS

The accompanying drawings, which are included to provide a further understanding of the disclosure, are incorporated in and constitute a part of this application, illustrate embodiments of the disclosure and together with the description serve to explain principles of the disclosure.

FIG. 1 illustrates a schematic circuit diagram of an organic light emitting display device in accordance with the present disclosure.

FIG. 2 illustrates a cross-sectional view of an organic light emitting display device as an example of an organic light emitting device in accordance with an example embodiment of the present disclosure.

FIG. 3 illustrates a cross-sectional view of an organic light emitting diode having a single emitting part in accordance with an example embodiment of the present disclosure.

FIG. 4 illustrates a cross-sectional view of an organic light emitting display device in accordance with another example embodiment of the present disclosure.

FIG. 5 illustrates a cross-sectional view of an organic light emitting diode having a double-stack structure in accordance with another example embodiment of the present disclosure.

FIG. 6 illustrates a cross-sectional view of an organic light emitting diode having a triple-stack structure in accordance with another example embodiment of the present disclosure.

### DETAILED DESCRIPTION

Reference will now be made in detail to aspects of the disclosure, examples of which are illustrated in the accompanying drawings. Wherever possible, the same reference numbers will be used throughout the drawings to refer to the same or like parts.

The present disclosure relates to an organic emitting diode and/or an organic light emitting device where a first electron transport layer including a first electron transporting material substituted with at least one spiro-structured fluorene moiety with beneficial thermal resistant property is disposed adjacently to an emitting material layer and a second electron layer including a second electron transporting material substituted with at least one anthracene moiety with beneficial electron mobility property is disposed between the first electron transport layer and a second electrode, so that the diode and/or the device can improve their high temperature stability and maximize their luminous intensity and luminous properties. The organic light emitting diode can be applied to an organic light emitting device such as an organic light emitting display device or an organic light emitting illumination device.

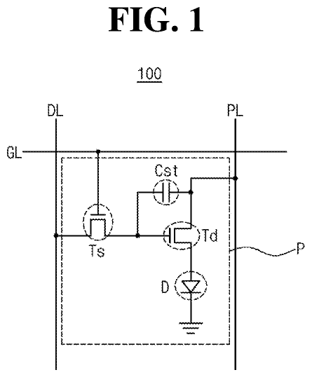

FIG. 1 illustrates a schematic circuit diagram of an organic light emitting display device in accordance with the present disclosure. As illustrated in FIG. 1, a gate line GL, a data line DL and power line PL, each of which crosses each other to define a pixel region P, in an organic light emitting display device 100. A switching thin film transistor Ts, a driving thin film transistor Td, a storage capacitor Cst and an organic light emitting diode D are disposed within the pixel region P. The pixel region P may include a red (R) pixel region, a green (G) pixel region and a blue (B) pixel region. However, embodiments of the present disclosure are not limited to such examples.

The switching thin film transistor Ts is connected to the gate line GL and the data line DL. The driving thin film transistor Td and the storage capacitor Cst are connected between the switching thin film transistor Ts and the power line PL. The organic light emitting diode D is connected to the driving thin film transistor Td. When the switching thin film transistor Ts is turned on by a gate signal applied to the gate line GL, a data signal applied to the data line DL is applied to a gate electrode of the driving thin film transistor Td and one electrode of the storage capacitor Cst through the switching thin film transistor Ts.

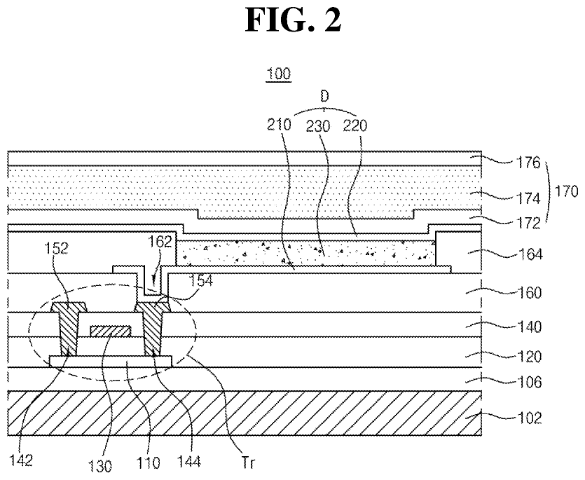

The driving thin film transistor Td is turned on by the data signal applied to the gate electrode 130 (FIG. 2) so that a current proportional to the data signal is supplied from the power line PL to the organic light emitting diode D through the driving thin film transistor Td. And then, the organic light emitting diode D emits light having a luminance proportional to the current flowing through the driving thin film transistor Td. In this case, the storage capacitor Cst is charged with a voltage proportional to the data signal so that the voltage of the gate electrode in the driving thin film transistor Td is kept constant during one frame. Therefore, the organic light emitting display device can display a desired image.

FIG. 2 illustrates a schematic cross-sectional view of an organic light emitting display device in accordance with an example embodiment of the present disclosure. As illustrated in FIG. 2, the organic light emitting display device 100 includes a substrate 102, a thin-film transistor Tr on the substrate 102, and an organic light emitting diode D connected to the thin film transistor Tr. As an example, the substrate 102 can include a red pixel region, a green pixel region and a blue pixel region and an organic light emitting diode D can be located in each pixel region. Each of the organic light emitting diodes D emitting red, green and blue light, respectively, is located correspondingly in the red pixel region, the green pixel region and the blue pixel region.

The substrate 102 can include, but is not limited to, glass, thin flexible material and/or polymer plastics. For example, the flexible material can be selected from, but is not limited to, the group consisting of polyimide (PI), polyethersulfone (PES), polyethylenenaphthalate (PEN), polyethylene terephthalate (PET), polycarbonate (PC) and/or combinations thereof. The substrate 102, on which the thin film transistor Tr and the organic light emitting diode D are arranged, forms an array substrate.

A buffer layer 106 can be disposed on the substrate 102. The thin film transistor Tr can be disposed on the buffer layer 106. The buffer layer 106 can be omitted.

A semiconductor layer 110 is disposed on the buffer layer 106. In one example embodiment, the semiconductor layer 110 can include, but is not limited to, oxide semiconductor materials. In this case, a light-shield pattern may be disposed under the semiconductor layer 110, and the light-shield pattern can prevent light from being incident toward the semiconductor layer 110, and thereby, preventing or reducing the semiconductor layer 110 from being degraded by the light. Alternatively, the semiconductor layer 110 can include polycrystalline silicon. In this case, opposite edges of the semiconductor layer 110 can be doped with impurities.

A gate insulating layer 120 including an insulating material is disposed on the semiconductor layer 110. The gate insulating layer 120 can include, but is not limited to, an inorganic insulating material such as silicon oxide (SiOₓ, wherein 0<x≤2) or silicon nitride (SiNₓ, wherein 0<x≤2).

A gate electrode 130 made of a conductive material such as a metal is disposed on the gate insulating layer 120 so as to correspond to a center of the semiconductor layer 110. While the gate insulating layer 120 is disposed on a whole area of the substrate 102 as shown in FIG. 2, the gate insulating layer 120 may be patterned identically as the gate electrode 130.

An interlayer insulating layer 140 including an insulating material is disposed on the gate electrode 130 with and covers an entire surface of the substrate 102. The interlayer insulating layer 140 can include, but is not limited to, an inorganic insulating material such as silicon oxide (SiOₓ) or silicon nitride (SiNₓ), or an organic insulating material such as benzocyclobutene or photo-acryl.

The interlayer insulating layer 140 has first and second semiconductor layer contact holes 142 and 144 that expose or do not cover a portion of the surface nearer to the opposing ends than to a center of the semiconductor layer 110. The first and second semiconductor layer contact holes 142 and 144 are disposed on opposite sides of the gate electrode 130 and spaced apart from the gate electrode 130. The first and second semiconductor layer contact holes 142 and 144 are formed within the gate insulating layer 120 in FIG. 2. Alternatively, the first and second semiconductor layer contact holes 142 and 144 can be formed only within the interlayer insulating layer 140 when the gate insulating layer 120 is patterned identically as the gate electrode 130.

A source electrode 152 and a drain electrode 154, which are made of conductive material such as a metal, are disposed on the interlayer insulating layer 140. The source electrode 152 and the drain electrode 154 are spaced apart from each other on opposing sides of the gate electrode 130, and contact both sides of the semiconductor layer 110 through the first and second semiconductor layer contact holes 142 and 144, respectively.

The semiconductor layer 110, the gate electrode 130, the source electrode 152 and the drain electrode 154 constitute the thin film transistor Tr, which acts as a driving element. The thin film transistor Tr in FIG. 2 has a coplanar structure in which the gate electrode 130, the source electrode 152 and the drain electrode 154 are disposed on the semiconductor layer 110. Alternatively, the thin film transistor Tr can have an inverted staggered structure in which a gate electrode is disposed under a semiconductor layer and a source and drain electrodes are disposed on the semiconductor layer. In this case, the semiconductor layer can include amorphous silicon.

The gate line GL and the data line DL, which cross each other to define a pixel region P, and a switching element Ts, which is connected to the gate line GL and the data line DL, can be further formed in the pixel region P. The switching element Ts is connected to the thin film transistor Tr, which is a driving element. In addition, the power line PL is spaced apart in parallel from the gate line GL or the data line DL. The thin film transistor Tr may further include a storage capacitor Cst configured to constantly keep a voltage of the gate electrode 130 for one frame.

A passivation layer 160 is disposed on the source and drain electrodes 152 and 154. The passivation layer 160 covers the thin film transistor Tr on the whole substrate 102. The passivation layer 160 has a flat top surface and a drain contact hole 162 that exposes or does not cover the drain electrode 154 of the thin film transistor Tr. While the drain contact hole 162 is disposed on the second semiconductor layer contact hole 144, it may be spaced apart from the second semiconductor layer contact hole 144.

The organic light emitting diode (OLED) D includes a first electrode 210 that is disposed on the passivation layer 160 and connected to the drain electrode 154 of the thin film transistor Tr. The OLED D further includes an emissive layer 230 and a second electrode 220 each of which is disposed sequentially on the first electrode 210.

The first electrode 210 is disposed in each pixel region. The first electrode 210 can be an anode and include conductive material having relatively high work function value. For example, the first electrode 210 can include a transparent conductive oxide (TCO). More particularly, the first electrode 210 can include, but is not limited to, indium tin oxide (ITO), indium zinc oxide (IZO), indium tin zinc oxide (ITZO), tin oxide (SnO), zinc oxide (ZnO), indium cerium oxide (ICO), aluminum doped zinc oxide (AZO), and/or the like.

In one example embodiment, when the organic light emitting display device 100 is a bottom-emission type, the first electrode 210 can have a single-layered structure of the TCO. Alternatively, when the organic light emitting display device 100 is a top-emission type, a reflective electrode or a reflective layer may be disposed under the first electrode 210. For example, the reflective electrode or the reflective layer can include, but is not limited to, silver (Ag) or aluminum-palladium-copper (APC) alloy. In the OLED D of the top-emission type, the first electrode 210 can have a triple-layered structure of ITO/Ag/ITO or ITO/APC/ITO.

In addition, a bank layer 164 is disposed on the passivation layer 160 in order to cover edges of the first electrode 210. The bank layer 164 exposes or does not cover a center of the first electrode 210 corresponding to each pixel region. The bank layer 164 may be omitted.

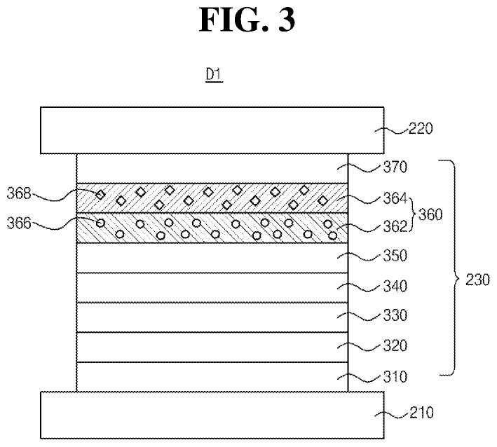

An emissive layer 230 is disposed on the first electrode 210. In one example embodiment, the emissive layer 230 can have a single-layered structure of an emitting material layer (EML). Alternatively, the emissive layer 230 can have a multiple-layered structure of a hole injection layer (HIL), a hole transport layer (HTL), an electron blocking layer (EBL), an EML, a hole blocking layer (HBL), an electron transport layer (ETL), an electron injection layer (EIL) and/or a charge generation layer (CGL) (FIGS. 3, 5 and 6). In one aspect, the emissive layer 230 can have a single emitting part. Alternatively, the emissive layer 230 can have multiple emitting parts to form a tandem structure.

The emissive layer 230 can include a first electron transport layer disposed adjacently to the EML and a second electron transport layer disposed between the first electron transport layer and the second electrode 220 so that the OLED can improve its high temperature stability and maintain light intensity emitted from the EML.

The second electrode 220 is disposed on the substrate 102 above which the emissive layer 230 is disposed. The second electrode 220 can be disposed on a whole display area. The second electrode 220 can include a conductive material with a relatively low work function value compared to the first electrode 210. The second electrode 220 can be a cathode providing electrons. For example, the second electrode 220 can include at least one of, but is not limited to, aluminum (Al), magnesium (Mg), calcium (Ca), silver (Ag), alloy thereof and/or combinations thereof such as aluminum-magnesium alloy (Al—Mg). When the organic light emitting display device 100 is a top-emission type, the second electrode 220 is thin so as to have light-transmissive (semi-transmissive) property.

In addition, an encapsulation film 170 can be disposed on the second electrode 220 in order to prevent or reduce outer moisture from penetrating into the OLED D. The encapsulation film 170 can have, but is not limited to, a laminated structure of a first inorganic insulating film 172, an organic insulating film 174 and a second inorganic insulating film 176. The encapsulation film 170 can be omitted.

A polarizing plate can be attached onto the encapsulation film 170 to reduce reflection of external light. For example, the polarizing plate may be a circular polarizing plate. When the organic light emitting display device 100 is a bottom-emission type, the polarizer can be disposed under the substrate 102. Alternatively, when the organic light emitting display device 100 is a top-emission type, the polarizer can be disposed on the encapsulation film 170. In addition, a cover window can be attached to the encapsulation film 170 or the polarizing plate. In this case, the substrate 102 and the cover window may have a flexible property, thus the organic light emitting display device 100 may be a flexible display device.

The OLED D is described in more detail. FIG. 3 illustrates a schematic cross-sectional view of an organic light emitting diode having a single emitting part in accordance with an example embodiment of the present disclosure. As illustrated in FIG. 3, the organic light emitting diode (OLED) D1 in accordance with the present disclosure includes a first electrode 210 and a second electrode 220 facing each other and an emissive layer 230 disposed between the first electrode 210 and the second electrode 220. The organic light emitting display device 100 includes a red pixel region, a green pixel region and a blue pixel region, and the OLED D1 can be disposed in the red pixel region, the green pixel region and/or the blue pixel region.

In an example embodiment, the emissive layer 230 includes an emitting material layer (EML) 340 disposed between the first and second electrodes 210 and 220. Also, the emissive layer 230 can include at least one of an HTL 320 disposed between the first electrode 210 and the EML 340 and an ETL 360 disposed between the second electrode 220 and the EML 340. In addition, the emissive layer 230 can further include at least one of an HIL 310 disposed between the first electrode 210 and the HTL 320 and an EIL 370 disposed between the second electrode 220 and the ETL 360. Alternatively, the emissive layer 230 can further comprise a first exciton blocking layer, i.e. an EBL 330 disposed between the HTL 320 and the EML 340 and/or a second exciton blocking layer, i.e. a HBL 350 disposed between the EML 340 and the ETL 360.

The first electrode 210 can be an anode that provides a hole into the EML 340. The first electrode 210 can include a conductive material having a relatively high work function value, for example, a transparent conductive oxide (TCO). In an example embodiment, the first electrode 210 can include, but is not limited to, ITO, IZO, ITZO, SnO, ZnO, ICO, AZO, and the like.

The second electrode 220 can be a cathode that provides an electron into the EML 340. The second electrode 220 can include a conductive material having a relatively low work function values, i.e., a highly reflective material such as Al, Mg, Ca, Ag, and/or alloy thereof and/or combinations thereof such as Al—Mg. For example, each of the first electrode 210 and the second electrode 220 can have, but is not limited to, a thickness of about 10 nm to about 300 nm.

The HIL 310 is disposed between the first electrode 210 and the HTL 320 and can improve an interface property between the inorganic first electrode 210 and the organic HTL 320. In one example embodiment, the HIL 310 can include, but is not limited to, 4,4′,4″-Tris(3-methylphenylamino)triphenylamine (MTDATA), 4,4′ ,4″-Tris(N,N-diphenyl-amino)triphenylamine (NATA), 4,4′,4″-Tris(N-(naphthalene-1-yl)-N-phenyl-amino)triphenylamine (1T-NATA), 4,4′,4″-Tris(N-(naphthalene-2-yl)-N-phenyl-amino)triphenylamine (2T-NATA), Copper phthalocyanine (CuPc), Tris(4-carbazoyl-9-yl-phenyl)amine (TCTA), N,N′-Diphenyl-N,N′-bis(1-naphthyl)-1,1′-biphenyl-4,4″-diamine (NPB; NPD), N,N′-Bis{4-[bis(3-methylphenyl)amino]phenyl}-N,N′-diphenyl-4,4′-biphenyldiamine (DNTPD) 1,4,5,8,9,11-Hexaazatriphenylenehexacarbonitrile (Dipyrazino [2,3-f:2′3′-h]quinoxaline-2,3,6,7,10,11-hexacarbonitrile; HAT-CN), 1,3,5-tris[4-(diphenylamino)phenyl]benzene (TDAPB), poly(3,4-ethylenedioxythiphene)polystyrene sulfonate (PEDOT/PSS), N-(biphenyl-4-yl)-9,9-dimethyl-N-(4-(9-phenyl-9H-carbazol-3-yl)phenyl)-9H-fluoren-2-amine, N,N′-diphenyl-N,N′-di[4-(N,N′-diphenyl-amino)phenyl]benzidine (NPNPB), MgF₂, CaF₂ and/or combinations thereof.

In an alternative embodiment, the HIL 310 can include a host of the above hole injecting material and/or hole transporting material below, and a P-type dopant. The P-type dopant can include, but is not limited to, HAT-CN, 2,3,5,6-tetrafluoro-7,7,8,8-tetracyanoquinodimethane (F4-TCNQ), 1,3,4,5,7,8-hexafluorotetracyanonaphthoquinodimethane (F6-TCNNQ), NPD9 and/or combinations thereof. The contents of the P-type dopant in the HIL 310 can be, but is not limited to, about 1 wt % to about 10 wt %. As an example, the HIL 310 can have a thickness of, but is not limited to, about 1 nm to about 100 nm. The HIL 310 can be omitted in compliance of the OLED D1 property.

The HTL 320 is disposed between the first electrode 210 and the EML 340. In one example embodiment, the HTL 320 can include, but is not limited to, N,N′-Diphenyl-N,N′-bis(3-methylphenyl)-1,1′-biphenyl-4,4′-diamine (TPD), NPB(NPD), DNTPD, N4,N4,N4′,N4*-Tetra[(1,1′-biphenyl)-4-yl]-(1,1′-biphenyl)-4,4′-diamine (BPBPA), 4,4′-bis(N-carbazolyl)-1,1′-biphenyl (CBP), Poly[N,N′-bis(4-butylphenyl)-N,N′-bis(phenyl)-benzidine](Poly-TPD), Poly [(9,9-dioctylfluorenyl-2,7-diyl)-co-(4,4′-(N-(4-sec-butylphenyl)diphenylamine))](TFB), Di-[4-(N,N-di-p-tolyl-amino)-phenyl]cyclohexane (TAPC), 3,5-Di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine), N-([1,1′-Biphenyl]-4-yl)-9,9-dimethyl-N-(4-(9-phenl-9H-carbazol-3-yl)phenyl)-9H-fluoren-2-amine and/or combinations thereof. As an example, the HTL 320 can have a thickness of, but is not limited to, about 20 nm to about 200 nm.

The EML 340 can include a host and a dopant (or an emitter). For example, the host can include a P-type host (hole-type host) and/or an N-type host (electron-type host).

In one example embodiment, the EML 340 can include a blue host and a blue dopant. As an example, the blue host can include, but is not limited to, 1,3-Bis(carbazol-9-yl)benzene (mCP), 9-(3-(9H-carbazol-9-yl)phenyl)-9H-carbazole-3-carbonitrile (mCP-CN), 3,3-Di(9H-carbazol-9-yl)biphenyl (mCBP), CBP-CN, 9-(3-(9H-Carbazol-9-yl)phenyl)-3-(diphenylphosphoryl)-9H-carbazole (mCPPO1) 3,5-Di(9H-carb azol-9-yl)biphenyl (Ph-mCP), Diphenyl-4-triphenylsilyl-phenylphosphine oxide (TSPO1), 9-(3′-(9H-carbazol-9-yl)[1,1′-biphenyl]-3-yl)-9H-pyrido [2,3-b]indole (CzBPCb), Bis(2-methylphenyl)diphenylsilane (UGH-1), 1,4-Bis(triphenylsilyl)benzene (UGH-2), 1,3-Bis(triphenylsilyl)benzene (UGH-3), 9,9-Spiorobifluoren-2-yl-diphenyl-phosphine oxide (SPPO1), 9,9′-(5-(Triphenylsilyl)-1,3-phenylene)bis(9H-carbazole) (SimCP), 2-methyl-9,10-bis(naphthalen-2-yl)anthracene (MADN) and/or combinations thereof.

The blue dopant can include at least one of blue phosphorescent material, blue fluorescent material and blue delayed fluorescent material. As an example, the blue dopant can include, but is not limited to, perylene, 4,4′-Bis[4-(di-p-tolylamino)styryl]biphenyl (DPAVBi), 4-(Di-p-tolylamino)-4-4′-[(di-p-tolylamino)styryl]stilbene (DPAVB), 4,4′-Bis[4-(diphenylamino) styryl]biphenyl (BDAVBi), 2,7-Bis(4-diphenylamino)s tyryl)-9,9-spirofluorene (spiro-DPVBi), [1,4-bis[2-[4-[N,N-di(p-tolyl)amino]phenyl]vinyl]benzene (DSB), 1-4-di-[4-(N,N-diphenyl)amino]styryl-benzene (DSA), 2,5,8,11-Tetra-tert-butylperylene (TB Pe), Bis(2-hydroxylphenyl)-pyridine)beryllium (Bepp₂), 9-(9-Phenylcarbazol-3-yl)-10-(naphthalen-1-yl)anthracene (PCAN), mer-Tris(1-phenyl-3-methylimidazolin-2-ylidene-C,C(2)'iridium(III) (mer-Ir(pmi)₃), fac-Tris(1,3-diphenyl-benzimidazolin-2-ylidene-C,C(2)′iridium(III) (fac-Ir(dpbic)₃), Bis(3,4,5-trifluoro-2-(2-pyridyl)pheny 1-(2-carboxypyridyl)iridium(III) (Ir(tfpd)₂pic), Tris(2-(4,6-difluorophenyl)pyridine))iridium(III) (Ir(Fppy)₃), Bis[2-(4,6-difluorophenyl)pyridinato-C², N](picolinato)iridium(III) (FIrpic), DABNA-1, DABNA-2, t-DABNA, v-DABNA and combinations thereof.

In another example embodiment, the EML³⁴⁰ can include a red host and a red dopant. As an example, the red host can include, but is not limited to, mCP-CN, CBP, mCBP, mCP, Bis[2-(diphenylphosphino)phenyl]ether oxide (DPEPO), 2,8-bis(diphenylphosphoryl)dibenzothiophene (PPT), 1,3,5-Tri [(3-pyridyl)-phen-3-yl]benzene (TmPyPB), 2,6-Di(9H-carbazol-9-yl)pyridine (PYD-2Cz), 2,8-di(9H-carbazol-9-yl)dibenzothiophene (DCzDBT), 3,5′-Di(carbazol-9-yl)-[1,1′-bipheyl]-3,5-dicarbonitrile (DCzTPA), 4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), TSPO1, 9-(9-phenyl-9H-carbazol-6-yl)-9H-carbazole (CCP), 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicarbazole, 9,9′-Diphenyl-9H,9′H-3,3′-bicarbazole (BCzPh), 1,3,5-Tris(carbazol-9-yl)benzene (TCP), TCTA, 4,4′-Bis(carbazole-9-yl)-2,2′-dimethylbipheyl (CDBP), 2,7-Bis(carbazole-9-yl)-9,9-dimethylfluorene (DMFL-CBP), 2,2′,7,7′-Tetrakis(carbazol-9-yl)-9,9-spirofluorene (Spiro-CBP), 3,6-Bis(carbazole-9-yl)-9-(2-ethyl-hexyl)-9H-carbazole (TCz1), BPBPA, 1,3,5-Tris(N-phenylbenzimidazol-2-yl)benzene (TPBi) and/or combinations thereof.

The red dopant can include at least one of red phosphorescent material, red fluorescent material and red delayed fluorescent material. As an example, the red dopant can include, but is not limited to, Bis[2-(4,6-dimethyl)phenylquinoline)](2,2,6,6-tetramethylheptane-3,5-dionate)iridium(III), Bis[2-(4-n-hexylphenyl)quinoline](acetylacetonate)iridium(III) (Hex-Ir(phq)₂(acac)), Tris[2-(4-n-hexylphenyl)quinoline]iridium(III) (Hex-Ir(phq)₃), Tris[2-phenyl-4-methylquinoline]iridium(III) (Ir(Mphq)₃), Bis(2-phenylquinoline)(2,2,6,6-tetramethylheptene-3,5-dionate)iridium(III) (Ir(dpm)PQ2), Bi (phenylisoquinoline)(2,2,6,6-tetramethylheptene-3,5-dionate)iridium(III) (Ir(dpm)(piq)₂), Bis(1-phenylisoquinoline)(acetylacetonate)iridium(III) (Ir(piq)₂(acac)), Bis[(4-n-hexylphenyl)is oquinoline](acetylacetonate)iridium(III) (Hex-Ir(piq)₂(acac)), Tris[2-(4-n-hexylphenyl)quinoline]iridium(III) (Hex-Ir(piq)₃), Tris(2-(3-methylphenyl)-7-methyl-quinolato)iridium (Ir(dmpq)₃), Bis[2-(2-methylphenyl)-7-methyl-quinoline](acetylacetonate)iridium(III) (Ir(dmpq)₂(acac)), Bis[2-(3,5-dimethylphenyl)-4-methyl-quinoline](acetylacetonate)iridium(III) (Ir(mphmq)₂(acac)), Tris(dibenzoylmethane)mono(1,10-phenanthroline)europium(III) (Eu(dbm)₃(phen)) and/or combinations thereof.

In another example embodiment, the EML 340 can include a green host and a green dopant. For example, the green host can include the blue host and/or the red host above. The green dopant can include at least one of green phosphorescent material, green fluorescent material and green delayed fluorescent material. As an example, the green dopant can include, but is not limited to, [Bis(2-phenylpyridine)](pyridyl-2-benzofuro [2,3-b]pyridine)iridium, Tris[2-phenylpyridine]iridium(III) (Ir(ppy)₃), fac-Tris(2-phenylpyridine)iridium(III) (fac-Ir(ppy)₃), Bis(2-phenylpyridine)(acetylacetonate)iridium(III) (Ir(Ppy)₂(acac)), Tris[2-(p-tolyl)pyridine]iridium(III) (Ir(mppy)₃), Bis(2-(naphthalene-2-yl)pyridine)(acetylacetonate)iridium(III) (Ir(npy)₂acac), Tris(2-phenyl-3-methyl-pyridine)iridium (Ir(3mppy)₃), fac-Tris(2-(3-p-xylyl)phenyl)pyridine iridium(III) (TEG) and/or combinations thereof.

The contents of the host including the P-type host and the N-type host in the EML 340 can be about 50 wt % to about 99 wt %, for example, about 80 wt % to about 95 wt %, and the contents of the dopant in the EML 340 can be about 1 wt % to about 50 wt %, for example, about 5 wt % to about 20 wt %, but is not limited thereto. When the EML 340 includes both the P-type host and the N-type host, the P-type host and the N-type host can be mixed, but is not limited to, with a weight ratio of about 4:1 to about 1:4, for example about 3:1 to about 1:3. As an example, the EML340 can have a thickness, but is not limited to, about 10 nm to about 200 nm.

The ETL 360 and the EIL 370 can be laminated sequentially between the EML 340 and the second electrode 220. An electron transporting material 366 or 368 included in the ETL 360 has high electron moibity so as to provide electrons stably with the EML 340 by fast electron transportation.

The ETL 360 can include a benzimidazole-based organic compound that can bind electrons transferred from the seocnd electrode 220 via the EIL 370 and that can inject electrons into the EML 340. The energy levels (e.g. LUMO energy level) between the ETL 360 including the benzimidazole-based organic compound and the EML 340 can be adjusted so as to inject electrons rapidly to the EML 340.

The ETL 360 includes a first electron transport layer (ETL1) 362 and a second electron transport layer (ETL2) 364 disposed sequentially between the EML 340 and the seocnd electrode. In other words, the ETL1 362 is disposed betwen the EML 340 and the second electrode 220 and the ETL2 364 is disposed between the ETL1 362 and the second electrode 220.

The ETL1 362 can include a first electron transporting material (ETM1) 366 of a benzimidazole-based organic compoind including a spiro fluorene strucutre with beneficial thermal resistant property. The ETML1 366 can be a benzimidazole-based organic compound having a structure of the following Chemical Formula 1:

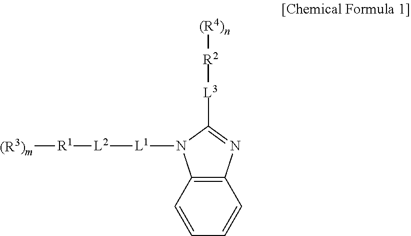

wherein, in the Chemical Formula 1,
each of R¹ and R² is independently an unsubstituted or substituted C₁-C₁₀ alkyl group, an unsubstituted or substituted C₆-C₃₀ aryl group or an unsubstituted or substituted C₃-C₃₀ hetero aryl group, and at least one of R¹ and R² is an unsubstituted or substituted spiro-fluorenyl group;
each of R³ and R⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R³ is identical to or different form each other when m is an integer of 2 or more, and where each R⁴ is identical to or different from each other when n is an integer of 2 or more;
each of L¹, L² and L³ is independently a single bond, an unsubstituted or substituted C₆-C₃₀ arylene group or an unsubstituted or substituted C₃-C₃₀ hetero arylene group; and
each of m and n is independently an integer of 0 to 7.

As used herein, the term “unsubstituted” means that hydrogen is directly linked to a carbon atom. “Hydrogen,” as used herein, may refer to protium, deuterium and tritium.

As used herein, “substituted” means that the hydrogen is replaced with a substituent. The substituent can comprise, but is not limited to, an unsubstituted or halogen-substituted C1-C₂₀ alkyl group, an unsubstituted or halogen-substituted C₁-C₂₀ alkoxy, halogen, a cyano group, a hydroxyl group, a carboxylic group, a carbonyl group, an amino group, a C₁-C₁₀ alkyl amino group, a C₆-C₃₀ aryl amino group, a C₃- C₃₀ hetero aryl amino group, a nitro group, a hydrazyl group, a sulfonate group, a C₁-C₁₀ alkyl silyl group, a C₁-C₁₀ alkoxy silyl group, a C₃-C₂₀ cyclo alkyl silyl group, a C₆-C₃₀ aryl silyl group, a C₃-C₃₀ hetero aryl silyl group, an unsubstituted or substituted C₆-C₃₀ aryl group, an unsubstituted or substituted C₃-C₃₀ hetero aryl group.

As used herein, the term “hetero” in terms such as “a hetero aryl group”, and “a hetero arylene group” and the likes means that at least one carbon atom, for example 1 to 5 carbons atoms, constituting an aliphatic chain, an alicyclic group or ring or an aromatic group or ring is substituted with at least one hetero atom selected from the group consisting of N, O, S and P.

The aryl group can independently include, but is not limited to, an unfused or fused aryl group such as phenyl, biphenyl, terphenyl, naphthyl, anthracenyl, pentalenyl, indenyl, indeno-indenyl, heptalenyl, biphenylenyl, indacenyl, phenalenyl, phenanthrenyl, benzo-phenanthrenyl, dibenzo-phenanthrenyl, azulenyl, pyrenyl, fluoranthenyl, triphenylenyl, chrysenyl, tetraphenylenyl, tetracenyl, pleiadenyl, picenyl, pentaphenylenyl, pentacenyl, fluorenyl, indeno-fluorenyl or spiro-fluorenyl.

The hetero aryl group can independently include, but is not limited to, an unfused or fused hetero aryl group such as pyrrolyl, pyridinyl, pyrimidinyl, pyrazinyl, pyridazinyl, triazinyl, tetrazinyl, imidazolyl, pyrazolyl, indolyl, iso-indolyl, indazolyl, indolizinyl, pyrrolizinyl, carbazolyl, benzo-carbazolyl, dibenzo-carbazolyl, indolo-carbazolyl, indeno-carbazolyl, benzo-furo-carbazolyl, benzo-thieno-carbazolyl, carbolinyl, quinolinyl, iso-quinolinyl, phthlazinyl, quinoxalinyl, cinnolinyl, quinazolinyl, quinolizinyl, purinyl, benzo-quinolinyl, benzo-iso-quinolinyl, benzo-quinazolinyl, benzo-quinoxalinyl, acridinyl, phenazinyl, phenoxazinyl, phenothiazinyl, phenanthrolinyl, perimidinyl, phenanthridinyl, pteridinyl, naphthyridinyl, furanyl, pyranyl, oxazinyl, oxazolyl, oxadiazolyl, triazolyl, dioxinyl, benzo-furanyl, dibenzo-furanyl, thiopyranyl, xanthenyl, chromenyl, iso-chromenyl, thioazinyl, thiophenyl, benzo-thiophenyl, dibenzo-thiophenyl, difuro-pyrazinyl, benzofuro-dibenzo-furanyl, benzothieno-benzo-thiophenyl, benzothieno-dibenzo-thiophenyl, benzothieno-benzo-furanyl, benzothieno-dibenzo-furanyl, xanthene-linked spiro acridinyl, dihydroacridinyl substituted with at least one C₁-C₁₀ alkyl and N-substituted spiro fluorenyl.

As an example, each of the C₁-C₁₀ alkyl group, the C₆-C₃₀ aryl group and the C₃-C₃₀ hetero aryl group of R¹ to R⁴ in Chemical Formula 1 can be independently unsubstituted or substituted with at least one of a C₁-C₁₀ alkyl group, a C₆-C₂₀ aryl group and a C₃-C₂₀ hetero aryl group, and each of the C₆-C₃₀ arylene group and the C₃-C₃₀ hetero arylene group of L¹ to L³ in Chemical Formula 1 can be independently unsubstituted or substituted with at least one C₁-C₁₀ alkyl group.

In one example embodiment, the ETM1 366 can include an organic compound where the Spiro fluorenyl group is substituted to the nitrogen atom constituting the benzimidazole moiety, or L¹ or L² that can be linked to the nitrogen atom. The ETM1 366 with such a linkage can have a structure of the following Chemical Formula 2:

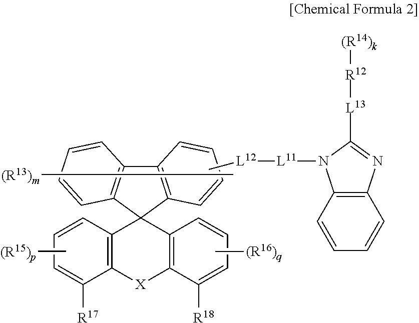

wherein, in the Chemical Formula 2,
R¹² is an unsubstituted or substituted C₁-C₁₀ alkyl group or an unsubstituted or substituted C₆-C₃₀ aryl group;
R¹³ is an unsubstituted or substituted C₁-C₁₀ alkyl group or an unsubstituted or substituted C₆-C₃₀ aryl group, and where each R¹³ is identical to or different from each other when m is an integer of 2 or more;
R¹⁴ is an unsubstituted or substituted C₁-C₁₀ alkyl group or an unsubstituted or substituted C₆-C₃₀ aryl group, and where each R¹⁴ is identical to or different from each other when k is an integer of 2 or more;
each of R¹⁵ and R¹⁶ is independently an C₁-C₁₀ alkyl group or an C₆-C₃₀ aryl group, where each R¹⁵ is identical to or different from each other when p is an integer of 2 or more, and where each R¹⁶ is identical to or different from each other when q is an integer of 2 or more;
each of R¹⁷ and R¹⁸ is independently hydrogen, an C₁-C₁₀ alkyl group or an C₆-C₃₀ aryl group, when X is a single bond; or one of R¹⁷ and R¹⁸, and X are further linked together to form an unsubstituted or substituted fused ring and the other of R¹⁷ and R¹⁸ is hydrogen, an C₁-C₁₀ alkyl group or an C₆-C₃₀ aryl group when X is nitrogen;
X is a single bond or nitrogen;
each of L¹¹, L¹² and L¹³ is independently a single bond or an unsubstituted or substituted C₆-C₃₀ arylene group;
m is a same as defined in Formula 1;
k is an integer of 0 to 3; and
each of p and q is an integer of 0 to 3.

As an example, each of C₁-C₁₀ alkyl group and the C₆-C₃₀ aryl group of R¹² to R¹⁸ in Chemical Formula 2 can be independently unsubstituted or substituted with at least one of a C₁-C₁₀ alkyl group and a C₆-C₂₀ aryl group, and the C₆-C₃₀ arylene group of L¹¹ to L¹³ in Chemical Formula 2 can be independently unsubstituted or substituted with at least one C₁-C₁₀ alkyl group. For example, when X in Chemical Formula 2 is nitrogen, the fluorene ring can form a spiro structure with an acridine moiety. In this case, the fused ring that can be formed by linking one of R¹⁷ and R¹⁸ to the nitrogen atom of X can include, but is not limited to, a pyrrole ring and an indole ring each of which can be independently unsubstituted or substituted with at least one C₁-C₅ alkyl group.

As an example, the C₁-C₁₀ alkyl group of R¹ to R⁴ in Chemical Formula 1 and R¹² to R¹⁸ in Chemical Formula 2 can be a C₁-05 alkyl group, and the C₆-C₃₀ aryl group of R¹ to R⁴ in Chemical Formula 1 and R¹² to R¹⁸ in Chemical Formula 2 can be selected from phenyl, biphenyl and naphthyl each of which can be independently unsubstituted or substituted with at least one of a C₁-C₁₀ alkyl group, a C₆-C₂₀ aryl group and a C₃-C₂₀ hetero aryl group. In addition, the C₆-C₃o arylene group of L¹ to L³ in Chemical Formula 1 and L¹¹ to L¹³ in Chemical Formula 2 can be selected from the group consisting of phenylene, biphenylene and a naphthylene each of which can be independently unsubstituted or substituted at least one (e.g., 1-2) C₁-C₅ alkyl group.

More particularly, the ETM1 366 can include at least one of, or can be selected from, but is not limited to, any benzimidazole-based organic compound of the following Chemical Formula 3:

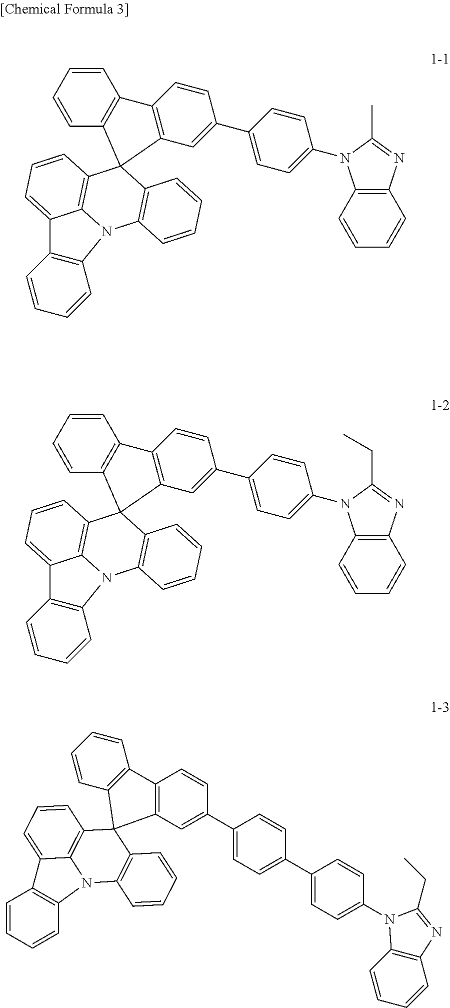 [SMILES: Cc1nc2ccccc2n1-c1ccc(-c2ccc3c(c2)C2(c4ccccc4-3)c3ccccc3-n3c4ccccc4c4cccc2c43)cc1 ; CCc1nc2ccccc2n1-c1ccc(-c2ccc3c(c2)C2(c4ccccc4-3)c3ccccc3-n3c4ccccc4c4cccc2c43)cc1 ; CCc1nc2ccccc2n1-c1ccc(-c2ccc(-c3ccc4c(c3)C3(c5ccccc5-4)c4ccccc4-n4c5ccccc5c5cccc3c54)cc2)cc1]

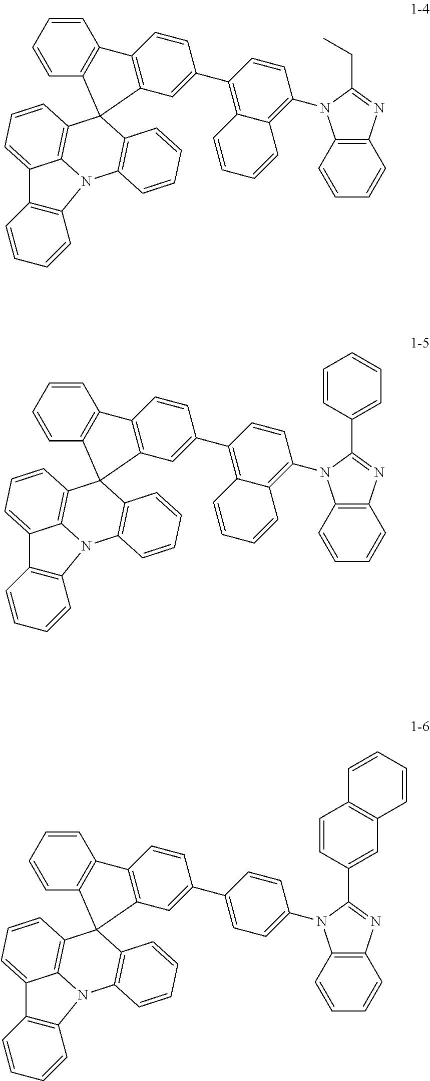 [SMILES: CCc1nc2ccccc2n1-c1ccc(-c2ccc3c(c2)C2(c4ccccc4-3)c3ccccc3-n3c4ccccc4c4cccc2c43)c2ccccc12 ; c1ccc(-c2nc3ccccc3n2-c2ccc(-c3ccc4c(c3)C3(c5ccccc5-4)c4ccccc4-n4c5ccccc5c5cccc3c54)c3ccccc23)cc1 ; c1ccc2c(c1)-c1ccc(-c3ccc(-n4c(-c5ccc6ccccc6c5)nc5ccccc54)cc3)cc1C21c2ccccc2-n2c3ccccc3c3cccc1c32]

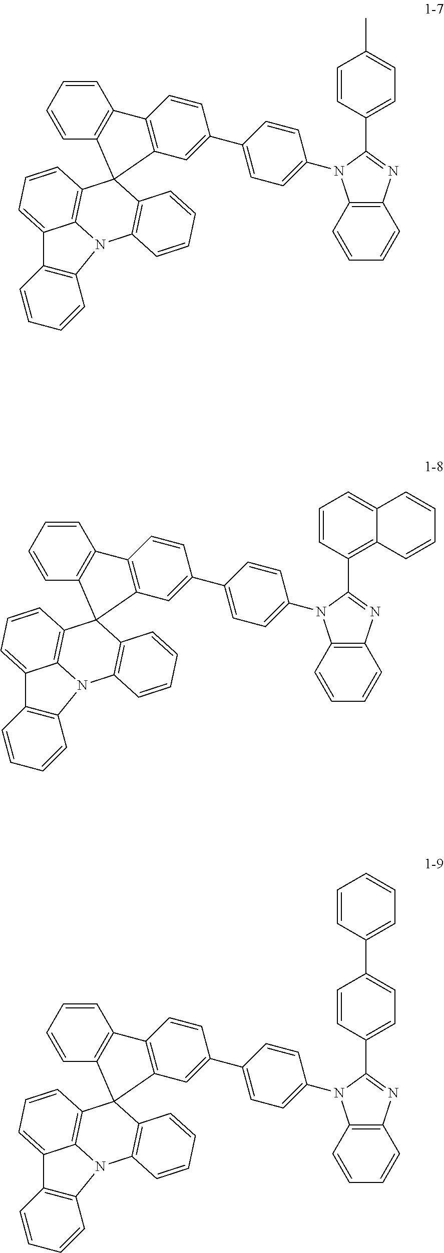 [SMILES: Cc1ccc(-c2nc3ccccc3n2-c2ccc(-c3ccc4c(c3)C3(c5ccccc5-4)c4ccccc4-n4c5ccccc5c5cccc3c54)cc2)cc1 ; c1ccc2c(c1)-c1ccc(-c3ccc(-n4c(-c5cccc6ccccc56)nc5ccccc54)cc3)cc1C21c2ccccc2-n2c3ccccc3c3cccc1c32 ; c1ccc(-c2ccc(-c3nc4ccccc4n3-c3ccc(-c4ccc5c(c4)C4(c6ccccc6-5)c5ccccc5-n5c6ccccc6c6cccc4c65)cc3)cc2)cc1]

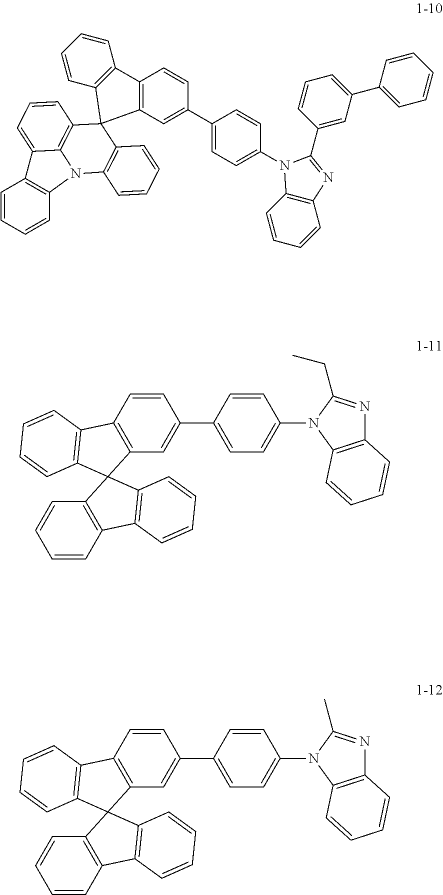 [SMILES: c1ccc(-c2cccc(-c3nc4ccccc4n3-c3ccc(-c4ccc5c(c4)C4(c6ccccc6-5)c5ccccc5-n5c6ccccc6c6cccc4c65)cc3)c2)cc1 ; CCc1nc2ccccc2n1-c1ccc(-c2ccc3c(c2)C2(c4ccccc4-c4ccccc42)c2ccccc2-3)cc1 ; Cc1nc2ccccc2n1-c1ccc(-c2ccc3c(c2)C2(c4ccccc4-c4ccccc42)c2ccccc2-3)cc1]

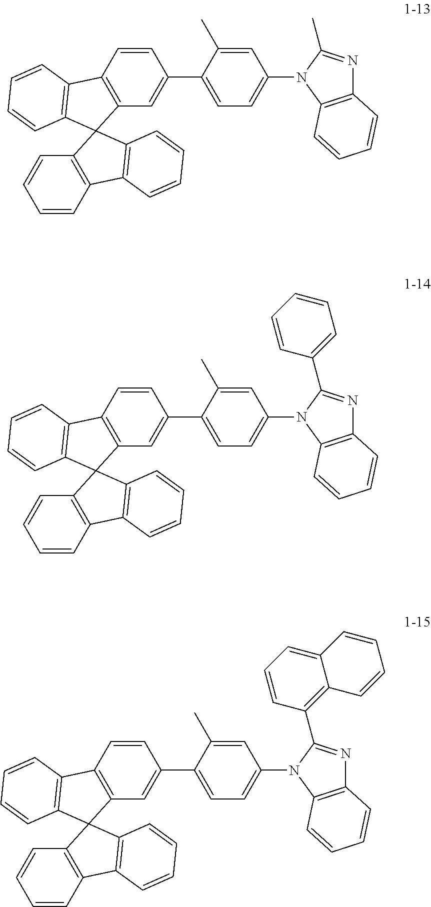 [SMILES: Cc1cc(-n2c(C)nc3ccccc32)ccc1-c1ccc2c(c1)C1(c3ccccc3-c3ccccc31)c1ccccc1-2 ; Cc1cc(-n2c(-c3ccccc3)nc3ccccc32)ccc1-c1ccc2c(c1)C1(c3ccccc3-c3ccccc31)c1ccccc1-2 ; Cc1cc(-n2c(-c3cccc4ccccc34)nc3ccccc32)ccc1-c1ccc2c(c1)C1(c3ccccc3-c3ccccc31)c1ccccc1-2]

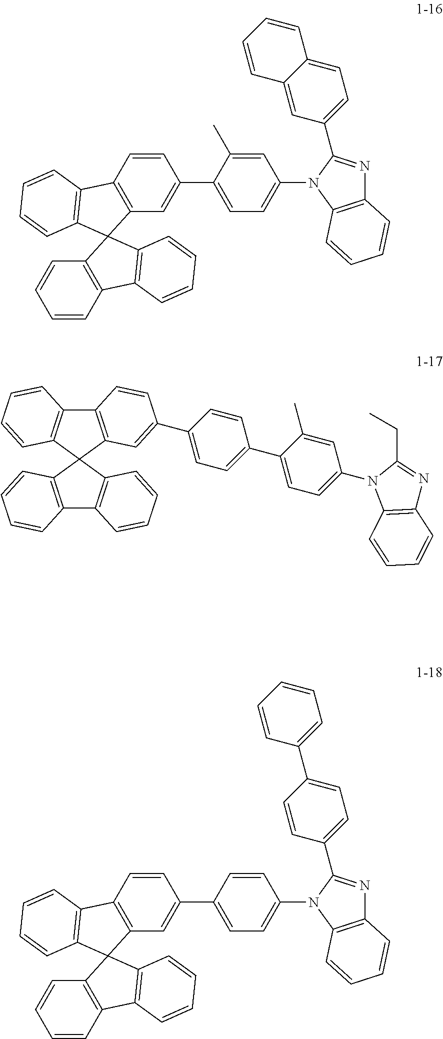 [SMILES: Cc1cc(-n2c(-c3ccc4ccccc4c3)nc3ccccc32)ccc1-c1ccc2c(c1)C1(c3ccccc3-c3ccccc31)c1ccccc1-2 ; CCc1nc2ccccc2n1-c1ccc(-c2ccc(-c3ccc4c(c3)C3(c5ccccc5-c5ccccc53)c3ccccc3-4)cc2)c(C)c1 ; c1ccc(-c2ccc(-c3nc4ccccc4n3-c3ccc(-c4ccc5c(c4)C4(c6ccccc6-c6ccccc64)c4ccccc4-5)cc3)cc2)cc1]

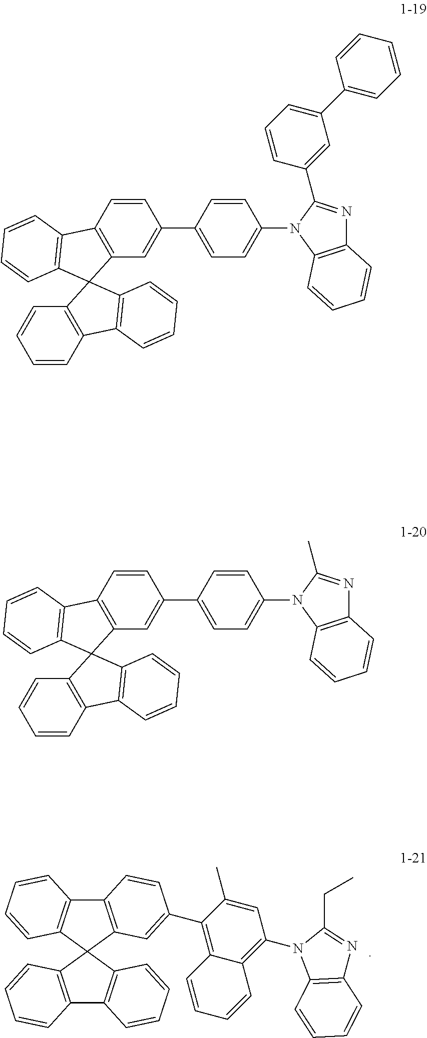 [SMILES: c1ccc(-c2cccc(-c3nc4ccccc4n3-c3ccc(-c4ccc5c(c4)C4(c6ccccc6-c6ccccc64)c4ccccc4-5)cc3)c2)cc1 ; Cc1nc2ccccc2n1-c1ccc(-c2ccc3c(c2)C2(c4ccccc4-c4ccccc42)c2ccccc2-3)cc1 ; CCc1nc2ccccc2n1-c1cc(C)c(-c2ccc3c(c2)C2(c4ccccc4-c4ccccc42)c2ccccc2-3)c2ccccc12]

The ETM1 366 having the structure of Chemical Formulae 1 to 3 is a benzimidazole-based organic compound substituted with bulky spiro-structured fluorenyl group. Compared to the below ETM2 368, the ETM1 366 has beneficial thermal resistant property due to the bulky group with identical or similar molecular weight. The interface between the EML 340 and the adjacently disposed ETL can be collapsed or the materials included in such layers can be degraded due to the high temperature heat generated in driving the OLED D1. However, it is possible to ensure high temperature stability by applying the ETM1 366 with excellent thermal resistance into the ETL1 362 disposed adjacently to the EML 340. It is possible to maintain light intensity emitted from the EML 340 even if the OLED D1 is driven for a long time. Accordingly, it is possible to realize OLED D1 with improved luminance and luminous lifespan.

The ETL2 364 can include the ETM2 368 including the anthracenyl group and having beneficial electron affinity and electron transporting property. The ETM2 368 can be a benzimidazole-based organic compound having a structure of the following Chemical Formula 4:

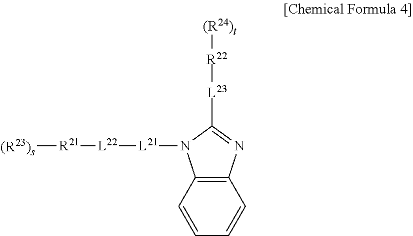

wherein, in the Chemical Formula 4,
each of R²¹ and R²² is independently an unsubstituted or substituted C₁-C₁₀ alkyl group, an unsubstituted or substituted C₆-C₃₀ aryl group or an unsubstituted or substituted C₃-C₃₀ hetero aryl group, and at least one of R²¹ and R²² is an unsubstituted or substituted anthracenyl group;
each of R²³ and R²⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R²³ is identical to or different form each other when s is an integer of 2 or more, and where each R²⁴ is identical to or different from each other when t is an integer of 2 or more;
each of L²¹, L²² and L²³ is independently a single bond, an unsubstituted or substituted C₆-C₃₀ arylene group or an unsubstituted or substituted C₃-C₃₀ hetero arylene group; and each of s and t is independently an integer of 0 to 9.

As an example, each of the C₁-C₁₀ alkyl group, the C₆-C₃₀ aryl group and the C₃-C₃₀ hetero aryl group of R²¹ to R²⁴ in Chemical Formula 4 can be independently unsubstituted or substituted with at least one of a C₁-C₁₀ alkyl group, a C₆-C₂₀ aryl group and a C₃-C₂₀ hetero aryl group, and each of the C₆-C₃₀ arylene group and the C₃-C₃₀ hetero arylene group of L²¹ to L²³ in Chemical Formula 4 can be independently unsubstituted or substituted with at least one C₁-C₁₀ alkyl group.

In one example embodiment, the ETM2 368 can include an organic compound where the anthracenyl group is substituted to the nitrogen atom constituting the benzimidazole moiety, or L²¹ or L²² that can be linked to the nitrogen atom. The ETM2 368 with such a linkage can have a structure of the following Chemical Formula 5:

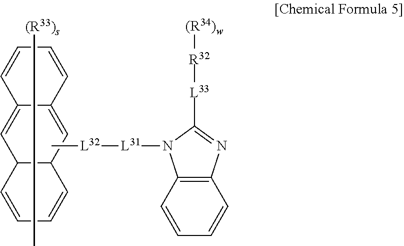

wherein, in the Chemical Formula 5,
R³² is an unsubstituted or substituted C₁-C₁₀ alkyl group or an unsubstitued or substituted C₆-C₃₀ aryl group;
R³³ is an unsubstituted or substituted C₁-C₁₀ alkyl group or an unsubstitued or substituted C₆-C₃₀ aryl group, and where each R³³ is identical to or different from each other when s is an integer of 2 or more;
R³⁴ is an unsubstituted or substituted C₆-C₃₀ aryl group, and where each R³⁴ is identical to or different from each other when w is an integer of 2 or more;
each of L³¹, L³² and L³³ is independently a single bond or an unsubstituted or substituted C₆-C₃₀ arylene group;
s is a same as defined in Formula 4; and
w is 0 or 1.

As an example, each of C₁-C₁₀ alkyl group and the C₆-C₃₀ aryl group of R³² to R³⁴ in Chemical Formula 5 can be independently unsubstituted or substituted with at least one of a C₁-C₁₀ alkyl group and a C₆-C₂₀ aryl group, and the C₆-C₃₀ arylene group of L³¹ to L³³ in Chemical Formula 5 can be independently unsubstituted or substituted with at least one C₁-C₁₀ alkyl group.

For example, the C₁-C₁₀ alkyl group of R²¹ to R²⁴ in Chemical Formula 4 and R³² to R³⁴ in Chemical Formula 5 can be a Cl-05 alkyl group, and the C₆-C₃₀ aryl group of R²¹ to R²⁴ in Chemical Formula 4 and R³² to R³⁴ in Chemical Formula 5 can be selected from phenyl, biphenyl and naphthyl each of which can be independently unsubstituted or substituted with at least one of a C₁-C₁₀ alkyl group, a C₆-C₂₀ aryl group and a C₃-C₂₀ hetero aryl group. In addition, the C₆-C₃o arylene group of L²¹ to L²³ in Chemical Formula 4 and L³¹ to L³³ in Chemical Formula 5 can be selected from the group consisting of phenylene, biphenylene and a naphthylene each of which can be independently unsubstituted or substituted at least one (e.g., 1-2) C₁-C₅ alkyl group.

More particularly, the ETM2 368 can include at least one of, or can be selected from, but is not limited to, any benzimidazole-based organic compound of the following Chemical Formula 6:

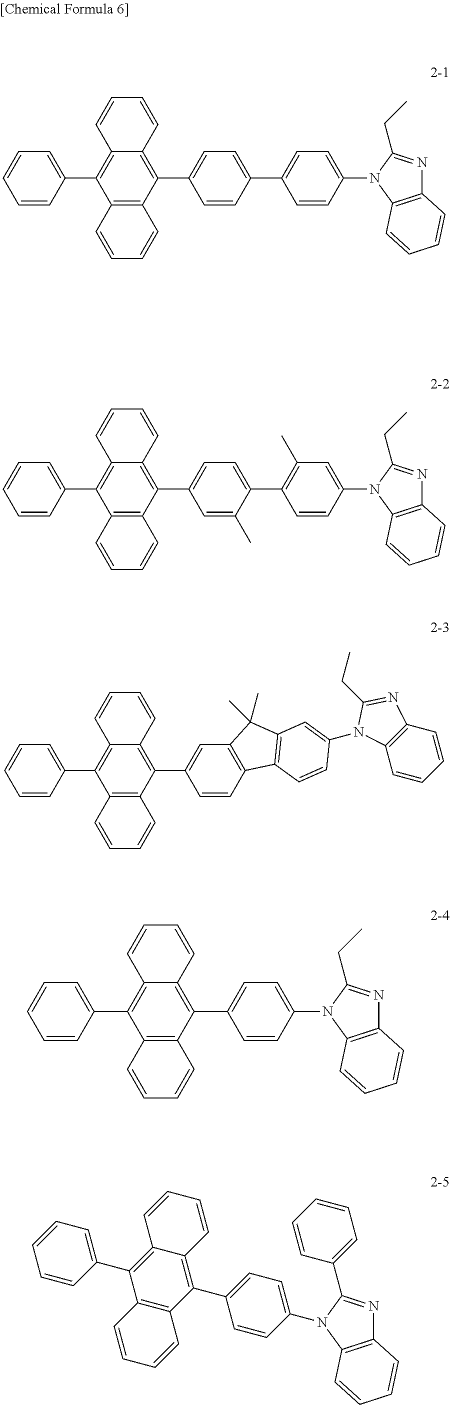 [SMILES: CCc1nc2ccccc2n1-c1ccc(-c2ccc(-c3c4ccccc4c(-c4ccccc4)c4ccccc34)cc2)cc1 ; CCc1nc2ccccc2n1-c1ccc(-c2ccc(-c3c4ccccc4c(-c4ccccc4)c4ccccc34)cc2C)c(C)c1 ; CCc1nc2ccccc2n1-c1ccc2c(c1)C(C)(C)c1cc(-c3c4ccccc4c(-c4ccccc4)c4ccccc34)ccc1-2 ; CCc1nc2ccccc2n1-c1ccc(-c2c3ccccc3c(-c3ccccc3)c3ccccc23)cc1 ; c1ccc(-c2c3ccccc3c(-c3ccc(-n4c(-c5ccccc5)nc5ccccc54)cc3)c3ccccc23)cc1]

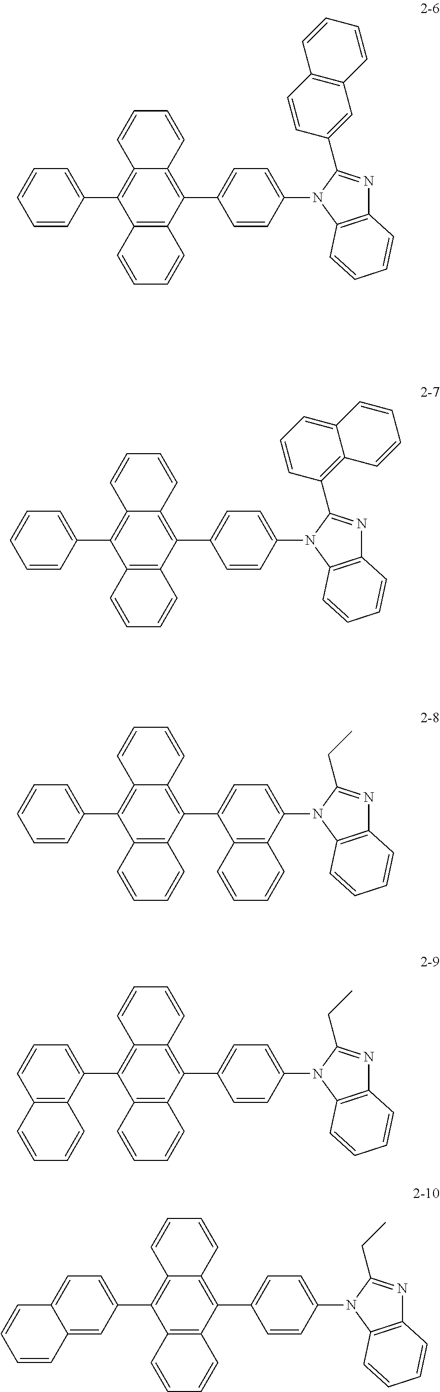 [SMILES: c1ccc(-c2c3ccccc3c(-c3ccc(-n4c(-c5ccc6ccccc6c5)nc5ccccc54)cc3)c3ccccc23)cc1 ; c1ccc(-c2c3ccccc3c(-c3ccc(-n4c(-c5cccc6ccccc56)nc5ccccc54)cc3)c3ccccc23)cc1 ; CCc1nc2ccccc2n1-c1ccc(-c2c3ccccc3c(-c3ccccc3)c3ccccc23)c2ccccc12 ; CCc1nc2ccccc2n1-c1ccc(-c2c3ccccc3c(-c3cccc4ccccc34)c3ccccc23)cc1 ; CCc1nc2ccccc2n1-c1ccc(-c2c3ccccc3c(-c3ccc4ccccc4c3)c3ccccc23)cc1]

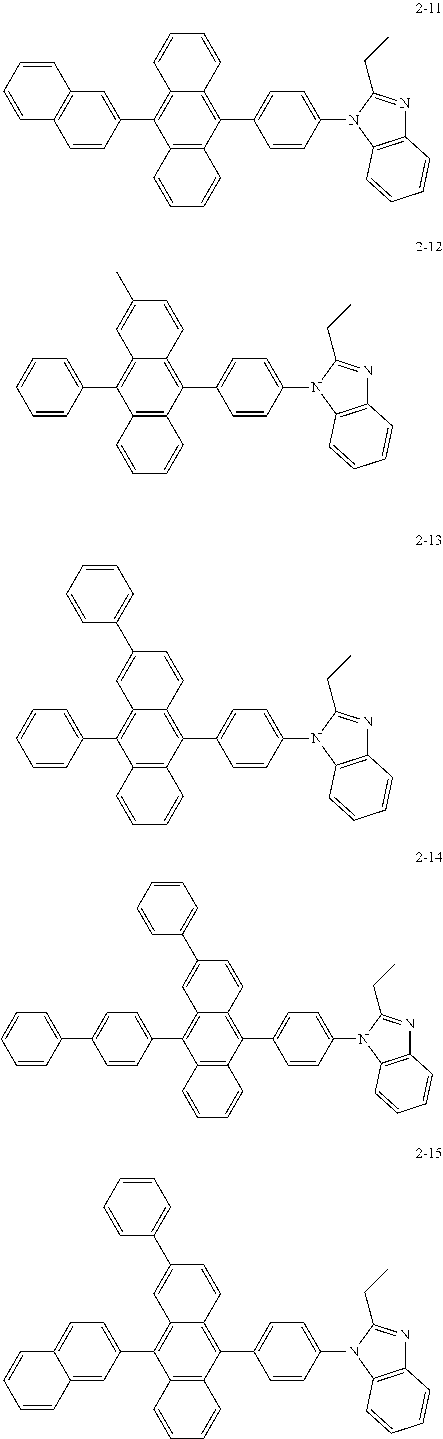 [SMILES: CCc1nc2ccccc2n1-c1ccc(-c2c3ccccc3c(-c3ccc4ccccc4c3)c3ccccc23)cc1 ; CCc1nc2ccccc2n1-c1ccc(-c2c3ccccc3c(-c3ccccc3)c3cc(C)ccc23)cc1 ; CCc1nc2ccccc2n1-c1ccc(-c2c3ccccc3c(-c3ccccc3)c3cc(-c4ccccc4)ccc23)cc1 ; CCc1nc2ccccc2n1-c1ccc(-c2c3ccccc3c(-c3ccc(-c4ccccc4)cc3)c3cc(-c4ccccc4)ccc23)cc1 ; CCc1nc2ccccc2n1-c1ccc(-c2c3ccccc3c(-c3ccc4ccccc4c3)c3cc(-c4ccccc4)ccc23)cc1]

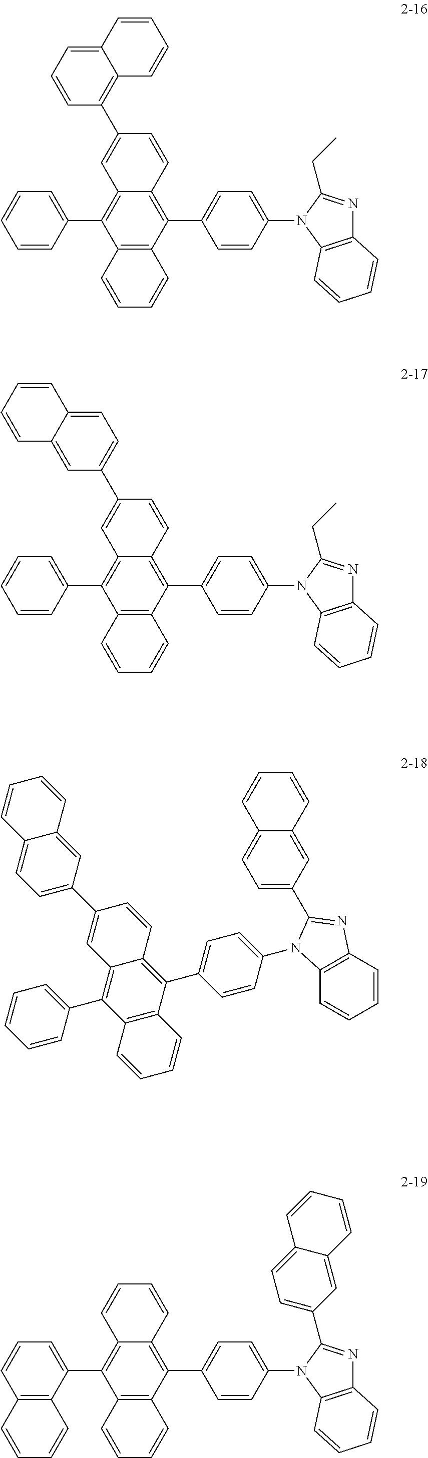 [SMILES: CCc1nc2ccccc2n1-c1ccc(-c2c3ccccc3c(-c3ccccc3)c3cc(-c4cccc5ccccc45)ccc23)cc1 ; CCc1nc2ccccc2n1-c1ccc(-c2c3ccccc3c(-c3ccccc3)c3cc(-c4ccc5ccccc5c4)ccc23)cc1 ; c1ccc(-c2c3ccccc3c(-c3ccc(-n4c(-c5ccc6ccccc6c5)nc5ccccc54)cc3)c3ccc(-c4ccc5ccccc5c4)cc23)cc1 ; c1ccc2cc(-c3nc4ccccc4n3-c3ccc(-c4c5ccccc5c(-c5cccc6ccccc56)c5ccccc45)cc3)ccc2c1]

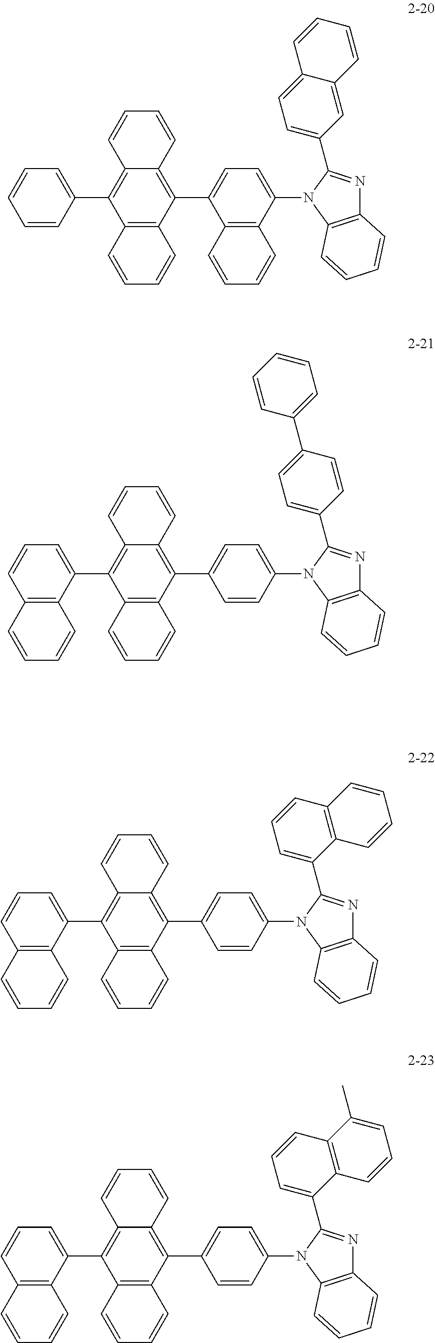 [SMILES: c1ccc(-c2c3ccccc3c(-c3ccc(-n4c(-c5ccc6ccccc6c5)nc5ccccc54)c4ccccc34)c3ccccc23)cc1 ; c1ccc(-c2ccc(-c3nc4ccccc4n3-c3ccc(-c4c5ccccc5c(-c5cccc6ccccc56)c5ccccc45)cc3)cc2)cc1 ; c1ccc2c(-c3c4ccccc4c(-c4ccc(-n5c(-c6cccc7ccccc67)nc6ccccc65)cc4)c4ccccc34)cccc2c1 ; Cc1cccc2c(-c3nc4ccccc4n3-c3ccc(-c4c5ccccc5c(-c5cccc6ccccc56)c5ccccc45)cc3)cccc12]

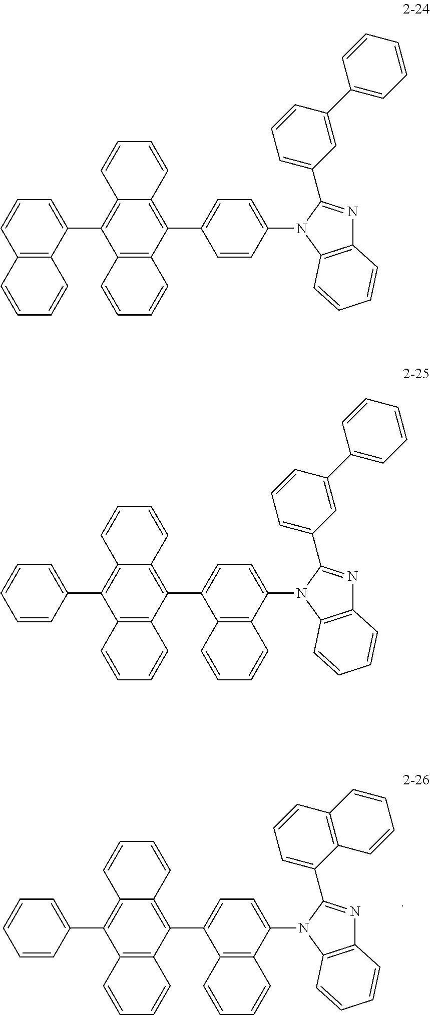 [SMILES: c1ccc(-c2cccc(-c3nc4ccccc4n3-c3ccc(-c4c5ccccc5c(-c5cccc6ccccc56)c5ccccc45)cc3)c2)cc1 ; c1ccc(-c2cccc(-c3nc4ccccc4n3-c3ccc(-c4c5ccccc5c(-c5ccccc5)c5ccccc45)c4ccccc34)c2)cc1 ; c1ccc(-c2c3ccccc3c(-c3ccc(-n4c(-c5cccc6ccccc56)nc5ccccc54)c4ccccc34)c3ccccc23)cc1]

The ETM2 368 having the structure of Chemical Formulae 4 to 6 includes the benzimidazole moiety with beneficial electron affinity. The ETM2 368 includes at least one anthracenyl group so that it can have appropriate energy level (e.g., LUMO energy level) for injecting electrons efficiently to the EML 340, and thus, electrons are injected rapidly to the EML 340. As holes via the HTL 320 and electrons via the ETL 360 are injected into the EML 340 in balance, the luminous properties of the OLED D1 can be improved.

The EIL 370 is disposed between the second electrode 220 and the ETL 360, and can improve physical properties of the second electrode 220 and therefore, can enhance the lifespan of the OLED D1. In one example embodiment, the EIL 370 may include, but is not limited to, an alkali metal halide or an alkaline earth metal halide such as LiF, CsF, NaF, BaF₂ and the like, and/or an organometallic compound such as Liq, lithium benzoate, sodium stearate, and the like.

As an example, each of the ETL 360 and the EIL 370 can have a thickness of, but is not limited to, about 1 nm to about 100 nm. Alternatively, the EIL 370 can be omitted.

In an alternative aspect, the electron transporting materials 366 and/or 368 and the electron injection material can be admixed to form a single-layered ETL-EIL. The electron transporting materials 366 and/or 368 and the electron injection material can be admixed with, but is not limited to, about 4:1 to about 1:4 by weight, for example, about 2:1 to about 1:2 by weight.

When holes are transferred to the second electrode 220 via the EML 340 and/or electrons are transferred to the first electrode 210 via the EML 340, the OLED D1 may have short lifespan and reduced luminous efficiency. In order to prevent those phenomena, the OLED D1 in accordance with this aspect of the present disclosure can have at least one exciton blocking layer adjacent to the EML 340.

For example, the OLED D1 can include the EBL 330 between the HTL 320 and the EML 340 so as to control and prevent electron transfers. In one example embodiment, the EBL 330 can include, but is not limited to, TCTA, Tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB, DCDPA, 2,8-bis(9-phenyl-9H-carbazol-3-yl)dibenzo[b,d]thiophene and/or combinations thereof.

In addition, the OLED D1 can further include the HBL 350 as a second exciton blocking layer between the EML 340 and the ETL 360 so that holes cannot be transferred from the EML 340 to the ETL 360. In one example embodiment, the HBL 350 can include, but is not limited to, at least one of oxadiazole-based compounds, triazole-based compounds, phenanthroline-based compounds, benzoxazole-based compounds, benzothiazole-based compounds, benzimidazole-based compounds, and triazine-based compounds.

For example, the HBL 350 can include material having a relatively low HOMO energy level compared to the luminescent materials in EML 340. The HBL 350 can include, but is not limited to, 2,9-Dimethyl-4,7-diphenyl-1,10-phenaathroline (BCP), Bis(2-methyl-8-quinolinolato-N1,O8)-(1,1′-biphenyl-4-olato)aluminum (B Alq), Tris-(8-hydroxyquinolinato) aluminum (Alq₃), 2-biphenyl-4-yl-5-(4-t-butylphenyl)-1,3,4-oxadiazole (PBD), spiro-PBD, Liq, Bis-4,5-(3,5-di-3-pyridylphenyl)-2-methylpyrimidine (B3PYMPM), DPEPO, 9-(6-(9H-carbazol-9-yl)pyridine-3-yl)-9H-3,9′-bicarbazole, TSPO1 and/or combinations thereof.

The HBL 350 can be omitted. As an example, each of the EBL 330 and the HBL 350 can have a thickness of, but is not limited to, about 1 nm to about 100 nm.

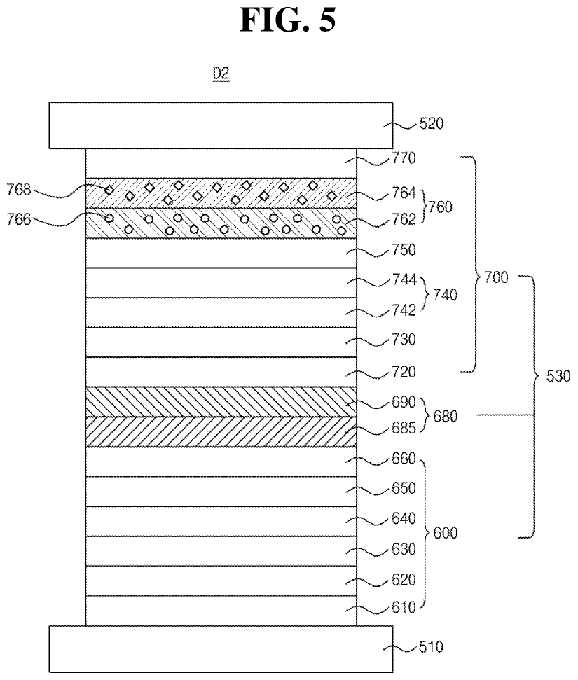

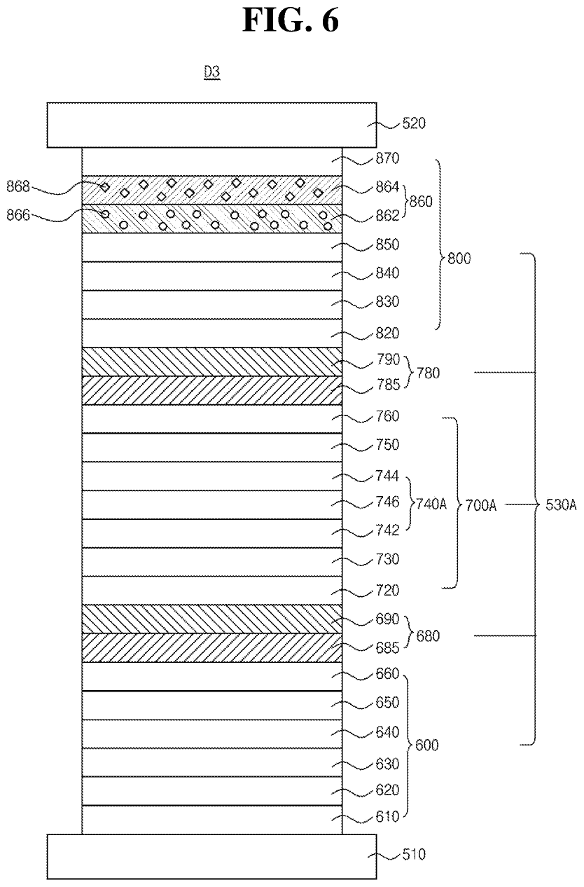

The OLED D1 with a single emitting part is shown in FIG. 3. However, the OLED can include multiple emitting parts (FIGS. 5 and 6) each of which can include an emitting material layer with identical or similar luminous peak ranges. As an example, each of the multiple emitting parts can emit red color, green color, or blue color.

The emitting part disposed adjacently to the second electrode 220 among the multiple emitting part has a structure where the ETL1 including the ETM1 and the ETL2 including the ETM2 are disposed sequentially between the emitting material layer and the second electrode. Accordingly, it is possible to realize a tandem-structured OLED with improving its high temperature stability, luminance and luminous lifespan.

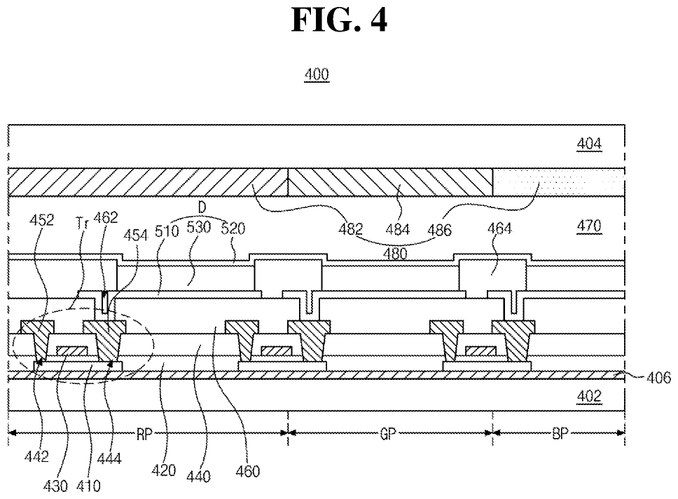

In another example embodiment, an organic light emitting display device can implement full-color including white color. FIG. 4 illustrates a schematic cross-sectional view of an organic light emitting display device in accordance with another example embodiment of the present disclosure.

As illustrated in FIG. 4, the organic light emitting display device 400 comprises a first substrate 402 that defines each of a red pixel region RP, a green pixel region GP and a blue pixel region BP, a second substrate 404 facing the first substrate 402, a thin film transistor Tr on the first substrate 402, an OLED D disposed between the first and second substrates 402 and 404 and emitting white (W) light and a color filter layer 480 disposed between the OLED D and the second substrate 404.

Each of the first and second substrates 402 and 404 can include, but is not limited to, glass, flexible material and/or polymer plastics. For example, each of the first substrate 402 and the second substrate 404 can be made of PI, PES, PEN, PET, PC and/or combinations thereof. The first substrate 402, on which a thin film transistor Tr and the OLED D are arranged, forms an array substrate.

A buffer layer 406 can be disposed on the first substrate 402. The thin film transistor Tr is disposed on the buffer layer 406 correspondingly to each of the red pixel region RP, the green pixel region GP and the blue pixel region BP. The buffer layer 406 can be omitted.

A semiconductor layer 410 is disposed on the buffer layer 406. The semiconductor layer 410 can be made of or include oxide semiconductor material or polycrystalline silicon.

A gate insulating layer 420 including an insulating material, for example, inorganic insulating material such as silicon oxide (SiOₓ, wherein 0<x≤2) or silicon nitride (SiNₓ, wherein 0<x≤2) is disposed on the semiconductor layer 410.

A gate electrode 430 made of a conductive material such as a metal is disposed over the gate insulating layer 420 so as to correspond to a center of the semiconductor layer 410. An interlayer insulating layer 440 including an insulating material, for example, inorganic insulating material such as SiOₓ or SiNₓ, or an organic insulating material such as benzocyclobutene or photo-acryl, is disposed on the gate electrode 430.

The interlayer insulating layer 440 has first and second semiconductor layer contact holes 442 and 444 that expose or do not cover a portion of the surface nearer to the opposing ends than to a center of the semiconductor layer 410. The first and second semiconductor layer contact holes 442 and 444 are disposed on opposite sides of the gate electrode 430 with spacing apart from the gate electrode 430.

A source electrode 452 and a drain electrode 454, which are made of or include a conductive material such as a metal, are disposed on the interlayer insulating layer 440. The source electrode 452 and the drain electrode 454 are spaced apart from each other with respect to the gate electrode 430. The source electrode 452 and the drain electrode 454 contact both sides of the semiconductor layer 410 through the first and second semiconductor layer contact holes 442 and 444, respectively.

The semiconductor layer 410, the gate electrode 430, the source electrode 452 and the drain electrode 454 constitute the thin film transistor Tr, which acts as a driving element.

Although not shown in FIG. 4, the gate line GL and the data line DL, which cross each other to define the pixel region P, and a switching element Ts, which is connected to the gate line GL and the data line DL, can be further formed in the pixel region P. The switching element Ts is connected to the thin film transistor Tr, which is a driving element. In addition, the power line PL is spaced apart in parallel from the gate line GL or the data line DL, and the thin film transistor Tr can further include the storage capacitor Cst configured to constantly keep a voltage of the gate electrode 430 for one frame.

A passivation layer 460 is disposed on the source electrode 452 and the drain electrode 454 and covers the thin film transistor Tr over the whole first substrate 402. The passivation layer 460 has a drain contact hole 462 that exposes or does not cover the drain electrode 454 of the thin film transistor Tr.

The OLED D is located on the passivation layer 460. The OLED D includes a first electrode 510 that is connected to the drain electrode 454 of the thin film transistor Tr, a second electrode 520 facing the first electrode 510 and an emissive layer 530 disposed between the first electrode 510 and the second electrode 520.

The first electrode 510 formed for each pixel region RP, GP, or BP can be an anode and can include a conductive material having relatively high work function value. For example, the first electrode 510 can include, but is not limited to, ITO, IZO, ITZO, SnO, ZnO, ICO, AZO, and/or the like. Alternatively, a reflective electrode or a reflective layer can be disposed under the first electrode 510. For example, the reflective electrode or the reflective layer can include, but is not limited to, Ag or APC alloy.

A bank layer 464 is disposed on the passivation layer 460 in order to cover edges of the first electrode 510. The bank layer 464 exposes or does not cover a center of the first electrode 510 corresponding to each of the red pixel RP, the green pixel GP and the blue pixel BP. The bank layer 464 can be omitted.

An emissive layer 530 that can include multiple emitting parts is disposed on the first electrode 510. As illustrated in FIGS. 5 and 6, the emissive layer 530 can include multiple emitting parts 600, 700, 700A, and 800 and at least one charge generation layer 680 and 780. Each of the emitting parts 600, 700, 700A and 800 includes at least one emitting material layer and can further include an HIL, an HTL, an EBL, an HBL, an ETL and/or an EIL.

The second electrode 520 can be disposed on the first substrate 402 above which the emissive layer 530 can be disposed. The second electrode 520 can be disposed over a whole display area, can include a conductive material with a relatively low work function value compared to the first electrode 510, and can be a cathode. For example, the second electrode 520 can include, but is not limited to, Al, Mg, Ca, Ag, alloy thereof, and/or combinations thereof such as Al—Mg.

Since the light emitted from the emissive layer 530 is incident to the color filter layer 480 through the second electrode 520 in the organic light emitting display device 400 in accordance with the second embodiment of the present disclosure, the second electrode 520 has a thin thickness so that the light can be transmitted.

The color filter layer 480 is disposed on the OLED D and includes a red color filter pattern 482, a green color filter pattern 484 and a blue color filter pattern 486 each of which is disposed correspondingly to the red pixel RP, the green pixel GP and the blue pixel BP, respectively. Although not shown in FIG. 4, the color filter layer 480 can be attached to the OLED D through an adhesive layer. Alternatively, the color filter layer 480 can be disposed directly on the OLED D.

In addition, an encapsulation film 470 can be disposed on the second electrode 520 in order to prevent or reduce outer moisture from penetrating into the OLED D. The encapsulation film 470 can have, but is not limited to, a laminated structure including a first inorganic insulating film, an organic insulating film and a second inorganic insulating film (170 in FIG. 2). In addition, a polarizing plate can be attached onto the second substrate 404 to reduce reflection of external light. For example, the polarizing plate can be a circular polarizing plate.

In FIG. 4, the light emitted from the OLED D is transmitted through the second electrode 520 and the color filter layer 480 is disposed on the OLED D. Alternatively, the light emitted from the OLED D is transmitted through the first electrode 510 and the color filter layer 480 can be disposed between the OLED D and the first substrate 402.

In addition, a color conversion layer may be formed or disposed between the OLED D and the color filter layer 480. The color conversion layer may include a red color conversion layer, a green color conversion layer and a blue color conversion layer each of which is disposed correspondingly to each pixel (RP, GP and BP), respectively, so as to convert the white (W) color light to each of a red, green and blue color lights, respectively. Alternatively, the organic light emitting display device 400 can comprise the color conversion film instead of the color filter layer 480.

As described above, the white (W) color light emitted from the OLED D is transmitted through the red color filter pattern 482, the green color filter pattern 484 and the blue color filter pattern 486 each of which is disposed correspondingly to the red pixel region RP, the green pixel region GP and the blue pixel region BP, respectively, so that red, green and blue color lights are displayed in the red pixel region RP, the green pixel region GP and the blue pixel region BP.

An OLED that can be applied into the organic light emitting display device will be described in more detail. FIG. 5 illustrates a schematic cross-sectional view of an organic light emitting diode having a tandem structure of two emitting parts. As illustrated in FIG. 5, the OLED D2 in accordance with the example embodiment of the present disclosure includes first and second electrodes 510 and 520 and an emissive layer 530 disposed between the first and second electrodes 510 and 520. The emissive layer 530 includes a first emitting part 600 disposed between the first and second electrodes 510 and 520, a second emitting part 700 disposed between the first emitting part 600 and the second electrode 520 and a charge generation layer (CGL) 680 disposed between the first and second emitting parts 600 and 700.

The first electrode 510 can be an anode and can include a conductive material having relatively high work function value such as TCO. For example, the first electrode 510 can include, but is not limited to, ITO, IZO, ITZO, SnO, ZnO, ICO, AZO, and/or the like. The second electrode 520 can be a cathode and can include a conductive material with a relatively low work function value. For example, the second electrode 520 can include, but is not limited to, Al, Mg, Ca, Ag, alloy thereof and/or combination thereof such as Al—Mg.

The first emitting part 600 includes a first EML (lower EML, EML1) 640. The first emitting part 600 can further include at least one of an HIL 610 disposed between the first electrode 510 and the EML1 640, a first HTL (bottom HTL, HTL1) 620 disposed between the HIL 610 and the EML1 640, and a bottom ETL (B-ETL) 660 disposed between the EML1 640 and the CGL 680. Alternatively, the first emitting part 600 can further include a first EBL (bottom EBL, EBL1) 630 disposed between the HTL1 620 and the EML1 640 and/or a first HBL (bottom HBL, HBL1) 650 disposed between the EML1 640 and the B-ETL 660.

The second emitting part 700 includes a second EML (upper EML, EML2) 740. The second emitting part 700 can further include at least one of a second HTL (upper HTL, HTL2) 720 disposed between the CGL 680 and the EML2 740, an upper ETL (U-ETL) 760 disposed between the second electrode 520 and the EML2 740 and an EIL 770 disposed between the second electrode 520 and the U-ETL 760. Alternatively, the second emitting part 700 can further include a second EBL (upper EBL, EBL2) 730 disposed between the HTL2 720 and the EML2 740 and/or a second HBL (upper HBL, HBL2) 750 disposed between the EML2 740 and the U-ETL 760.

One of the EML1 640 and the EML2 740 can emit blue color light, and the other of the EML1 640 and the EML2 740 can emit any colors with longer wavelength ranges than the blue color light, for example, red-green color light so that the OLED D2 can realize white (W) emission. Hereinafter, the OLED D2 where the EML1 640 emits blue color light and the EML2 740 emits red-green color light will be described in detail.

The HIL 610 is disposed between the first electrode 510 and the HTL1 620 and improves an interface property between the inorganic first electrode 510 and the organic HTL1 620. In one exemplary embodiment, the HIL 610 can include, but is not limited to, MTDATA, NATA, 1T-NATA, 2T-NATA, CuPc, TCTA, NPB (NPD), DNTPD, HAT-CN, TDAPB, PEDOT/PSS, N-(biphenyl-4-yl)-9,9-dimethyl-N-(4-(9-phenyl-9H-carbazol-3-yl)phenyl)-9H-fluoren-2-amine, NPNPB, MgF₂, CaF₂ and/or combinations thereof. Alternatively, the HIL 610 can include a host of the hole injecting material and/or the hole transporting material and a P-type dopant that can be HAT-CN, F4-TCNQ, F6-TCNNQ, NPD9 and/or combinations thereof. The HIL 610 can be omitted in compliance of the OLED D2 property.

In one example embodiment, each of the HTL1 620 and the HTL2 720 can include, but is not limited to, TPD, NPB (NPD), DNTPD, BPBPA, CBP, Poly-TPD, TFB, TAPC, DCDPA, N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4- amine, N-([1,1′-Biphenyl]-4-yl)-9,9-dimethyl-N-(4-(9-phen1-9H-carbazol-3-yl)phenyl)-9H-fluoren-2- amine and/or combination thereof.

Each of the B-ETL 660 and the U-ETL 760 facilitates electron transportation in each of the first emitting part 600 and the second emitting part 700, respectively. As an example, the B-ETL 660 can include at least one of oxadiazole-based compounds, triazole-based compounds, phenanthroline-based compounds, benzoxazole-based compounds, benzothiazole-based compounds, benzimidazole-based compound and triazine-based compounds.

For example, the B-ETL 660 can include, but is not limited to, Alq₃, PBD, spiro-PBD, Liq, TPBi, B Alq, 4,7-diphenyl-1,10-phenanthroline (Bphen), 2,9-Bis(naphthalene-2-yl)4,7-diphenyl-1,10-phenanthroline (NB phen), BCP, 3-(4-Biphenyl)-4-phenyl-5-tert-butylphenyl-1,2,4-triazole (TAZ), 4-(Naphthalen-l-yl)-3,5-diphenyl-4H-1,2,4-triazole (NTAZ), 1,3,5-Tri(p-pyrid-3-yl-phenyl)benzene (TpPyPB), 2,4,6-Tris(3′-(pyridin-3-yl)biphenyl-3-yl)1,3,5-triazine (TmPPPyTz), Poly[9,9-bis(3′-(N,N-dimethyl)-N-ethylammonium)-propyl)-2,7-fluorene]-alt-2,7-(9,9-dioctylfluorene)](PFNBr), Tris(phenylquinoxaline) (TPQ), TSPO1, 2-[4-(9,10-di-2-naphthalen-2-yl-2-anthracen-2-yl)phenyl]1-phenyl-1H-benzimidazole (ZADN) and/or combinations thereof.

The U-ETL 760 includes a first ETL (ETL1) 762 and a second ETL (ETL2) 764 that are located sequentially between the EML2 740 and the second electrode 520. The ETL1 762 disposed adjacently to the EML2 740 includes a first electron transporting material (ETM1) 766 of a benzimidazole-based organic compound having a structure of Chemical Formulae 1 to 3 and substituted with at least one spiro-structured fluorenyl group. The ETL2 764 disposed on the ETL1 762 includes a second electron transporting material (ETML2) 768 of a benzimidazole-based organic compound having a structure of Chemical Formulae 4 to 6 and substituted with at least one anthracenyl group. The ETL1 762 including the ETM1 766 with beneficial thermal resistant property is disposed adjacently to the EML2 740 and the ETL2 764 including the ETM2 768 with beneficial electron transporting property is disposed adjacently to the second electrode, so that the high temperature stability, light intensity, luminance and luminous lifespan of the OLED D2 can be improved.

The EIL 770 is disposed between the second electrode 520 and the U-ETL 760, and can improve physical properties of the second electrode 520 and therefore, can enhance the lifespan of the OLED D2. In one example embodiment, the EIL 770 can include, but is not limited to, an alkali metal halide or an alkaline earth metal halide such as LiF, CsF, NaF, BaF₂ and the like, and/or an organometallic Compound such as Liq, lithium benzoate, sodium stearate, and the like.

Each of the EBL1 630 and the EBL2 730 can independently include, but is not limited to, TCTA, Tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB , DCDPA, 2,8-bis (9-phenyl-9H-carbazol-3-yl)dibenzo[b,d]thiophene and/or combinations thereof, respectively.

Each of the HBL1 650 and the HBL2 750 can include, but is not limited to, at least one of oxadiazole-based compounds, triazole-based compounds, phenanthroline-based compounds, benzoxazole-based compounds, benzothiazole-based compounds, benzimidazole-based compounds, and triazine-based compounds. For example, each of the HBL1 650 and the HBL2 750 can independently include, but is not limited to, BCP, BAlq, Alq₃, PBD, spiro-PBD, Liq, B3PYMPM, DPEPO, 9-(6-(9H-carbazol-9-yl)pyridine-3-yl)-9H-3,9′-bicarbazole, TSPO1 and/or combinations thereof, respectively.

The CGL 680 is disposed between the first emitting part 600 and the second emitting part 700. The CGL 680 includes an N-type CGL (N-CGL) 685 disposed adjacently to the first emitting part 600 and a P-type CGL (P-CGL) 690 disposed adjacently to the second emitting part 700. The N-CGL 685 injects electrons to the EML1 640 of the first emitting part 600 and the P-CGL 690 injects holes to the EML2 740 of the second emitting part 700.

The N-CGL 685 can be an organic layer doped with an alkali metal such as Li, Na, K and Cs and/or an alkaline earth metal such as Mg, Sr, Ba and Ra. For example, the host in the N-CGL 685 can include, but is not limited to, Bphen and MTDATA. The contents of the alkali metal or the alkaline earth metal in the N-CGL 685 can be between about 0.01 wt % and about 30 wt %.

The P-CGL 690 can include, but is not limited to, inorganic material selected from the group consisting of WOₓ, MoOₓ, Be₂O_{3,} V₂O₅ and combinations thereof and/or organic material selected from the group consisting of NPD, DNTPD, HAT-CN, F4-TCNQ, F6-TCNNQ, TPD, N,N,N′,N′-tetranaphthalenyl-benzidine (TNB), TCTA, N,N′-dioctyl-3,4,9,10-perylenedicarboximide (PTCDI-C8) and/or combinations thereof. Alternatively, the P-CGL 690 can include a host of NPD, DNTPD, TPD, TNB, TCTA and combinations thereof, and a P-type dopant of F4-TCNQ, F6-TCNNQ, NPD-9 and combinations thereof. The contents of the P-type dopant in the P-CGL 690 can be, but is not limited to, about 1 wt % to about 30 wt %, for example, about 3 wt % to about 25 wt %.

The EML1 640 can be a blue EML. In this case, the EML1 640 can be a blue EML, a sky-blue EML or a deep-blue EML. The EML1 640 can include a blue host and a blue dopant. The blue dopant can include at least one of blue phosphorescent material, blue fluorescent material and blue delayed fluorescent material. The blue host and the blue dopant can be identical to materials with referring to FIG. 3.

The EML2 740 can include a first layer 742 disposed between the EBL2 730 and the HBL2 750 and a second layer 744 disposed between the first layer 742 and the HBL2 750. One of the first layer 742 and the second layer 744 can emit red color light and the other of the first layer 742 and the second layer 744 can emit green color light. Hereinafter, the EML2 740 where the first layer 742 emits a red color light and the second layer 744 emits a green color light will be described in detail.

The first layer 742 can include a red host and a red dopant. The red dopant can include at least one of red phosphorescent material, red fluorescent material and red delayed fluorescent material. The red host and the red dopant can be identical to materials with referring to FIG. 3.

The second layer 744 can include a green host and a green dopant. The green dopant can include at least one of green phosphorescent material, green florescent material and green delayed fluorescent material. The green host and the green dopant can be identical to materials with referring to FIG. 3.

As an example, the contents of the host in each of the EML1 640 and the EML2 740 can be, but is not limited to, between about 50 wt % and about 99 wt %, for example, about 80 wt % and about 95 wt %, and the contents of the dopant in each of the EML1 640 and the EML2 740 can be, but is not limited to, between about 1 wt % and about 50 wt %, for example, about 5 wt % and about 20 wt %. When each of the EML1 640 and the EML2 740 includes both the P-type host and the N-type host, the P-type host and the N-type host can be admixed, but is not limited to, with a weight ratio from about 4:1 to about 1:4, for example, about 3:1 to about 1:3. Alternatively, the EML2 740 can further include a third layer (746 in FIG. 6) that can emit yellow-green color light disposed between the first layer 742 and the second layer 744.

An OLED can have three or more emitting parts to form a tandem structure. FIG. 6 is a schematic cross-sectional view illustrating an organic light emitting diode in accordance with yet another example embodiment of the present disclosure.

As illustrated in FIG. 6, the OLED D3 includes first and second electrodes 510 and 520 facing each other and an emissive layer 530A disposed between the first and second electrodes 510 and 520. The emissive layer 530A includes a first emitting part 600 disposed between the first and second electrodes 510 and 520, a second emitting part 700A disposed between the first emitting part 600 and the second electrode 520, a third emitting part 800 disposed between the second emitting part 700A and the second electrode 520, a first charge generation layer (CGL1) 680 disposed between the first and second emitting parts 600 and 700A, and a second charge generation layer (CGL2) 780 disposed between the second and third emitting parts 700A and 800.

The first emitting part 600 includes a first EML (lower EML, EML1) 640. The first emitting part 600 can further include at least one of an HIL 610 disposed between the first electrode 510 and the EML1 640, a first HTL (lower HTL, HTL1) 620 disposed between the HIL 610 and the EML1 640, a bottom ETL (B-ETL) 660 disposed between the EML1 640 and the CGL 680. Alternatively, the first emitting part 600 can further comprise a first EBL (bottom EBL, EBL1) 630 disposed between the HTL1 620 and the EML1 640 and/or a first HBL (bottom HBL, HBL1) 650 disposed between the EML1 640 and the B-ETL 660.

The second emitting part 700A includes a second EML (middle EML, EML2) 740A. The second emitting part 700A can further include at least one of a second HTL (middle HTL, HTL2) 720 disposed between the CGL1 680 and the EML2 740A and a middle ETL (M-ETL) 760 disposed between the EML2 740A and the CGL2 780. Alternatively, the second emitting part 700A can further include a second EBL (middle EBL, EBL2) 730 disposed between the HTL2 720 and the EML2 740A and/or a second HBL (middle HBL, HBL2) 750 disposed between the EML2 740A and the M-ETL 760.

The third emitting part 800 includes a third EML (upper EML, EML3) 840. The third emitting part 800 can further include at least one of a third HTL (upper HTL, HTL3) 820 disposed between the CGL2 780 and the EML3 840, an upper ETL (U-ETL) 860 disposed between the second electrode 520 and the EML3 840 and an EIL 870 disposed between the second electrode 520 and the U-ETL 860. Alternatively, the third emitting part 800 can further comprise a third EBL (upper EBL, EBL3) 830 disposed between the HTL3 820 and the EML3 840 and/or a third HBL (upper HBL, HBL3) 850 disposed between the EML3 840 and the U-ETL 860.

At least one of the EML1 640, the EML2 740A and the EML3 840 can emit red-green color light, and another of the EML1 640, the EML2 740A and the EML3 840 can emit a blue color so that the OLED D3 can realize white emission. Hereinafter, the OLED where the EML2 740A emits red-green color will be described in detail.

Each of the HTL1 620, the HTL2 720 and the HTL3 820 can include independently, but is not limited to, TPD, NPB (NPD), DNTPD, BPBPA, CBP, Poly-TPD, TFB, TAPC, DCDPA, N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine, N-([1,1′-Biphenyl]-4-yl)-9,9-dimethyl-N-(4-(9-phen1-9H-carbazol-3-yl)phenyl)-9H-fluoren-2- amine and/or combinations thereof.

Each of the B-ETL 660, the M-ETL 760 and the U-ETL 760 provides electron transportation in each of the first emitting part 600, the second emitting part 700A and the third emitting part 800, respectively. As an example, each of the B-ETL 660 and the M-ELT 760 can include independently, but is not limited to, at least one of oxadiazole-based compounds, triazole-based compounds, phenanthroline-based compounds, benzoxazole-based compounds, benzothiazole-based compounds, benzimidazole-based compounds, triazine-based compounds.

For example, each of the B-ETL 660 and the M-ETL 760 can independently include, but is not limited to, Alq₃, PBD, spiro-PBD, Liq, TPBi, BAlq, Bphen, NBphen, BCP, TAZ, NTAZ, TpPyPB, TmPPPyTz, PFNBr, TPQ, TSPO1, ZADN and/or combinations thereof.

The U-ETL 860 includes a first ETL (ETL1) 862 and a second ETL (ETL2) 864 that are located sequentially between the EML3 840 and the second electrode 520. The ETL1 862 disposed adjacently to the EML3 840 includes a first electron transporting material (ETM1) 866 of a benzimidazole-based organic compound having a structure of Chemical Formulae 1 to 3 and substituted with at least one spiro-structured fluorenyl group. The ETL2 864 disposed on the ETL1 862 includes a second electron transporting material (ETML2) 868 of a benzimidazole-based organic compound having a structure of Chemical Formulae 4 to 6 and substituted with at least one anthracenyl group. The ETL1 862 including the ETM1 866 with beneficial thermal resistant property is disposed adjacently to the EML3 840 and the ETL2 864 including the ETM2 868 with beneficial electron transporting property is disposed adjacently to the second electrode, so that the high temperature stability, light intensity, luminance and luminous lifespan of the OLED D3 can be improved.

Each of the EBL1 630, the EBL2 730 and the EBL3 830 can independently include, but is not limited to, TCTA, Tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB, DCDPA, 2,8-bis(9-phenyl-9H-carbazol-3-yl)dibenzo[b,d]thiophene and/or combinations thereof, respectively.

Each of the HBL1 650, the HBL2 750 and the HBL3 850 can include independently at least one of oxadiazole-based compounds, triazole-based compounds, phenanthroline-based compounds, benzoxazole-based compounds, benzothiazole-based compounds, benzimidazole-based compounds, triazine-based compounds.

The CGL1 680 is disposed between the first emitting part 600 and the second emitting part 700A and the CGL2 780 is disposed between the second emitting part 700A and the third emitting part 800. The CGL1 680 includes a first N-type CGL (N-CGL1) 685 disposed adjacently to the first emitting part 600 and a first P-type CGL (P-CGL1) 690 disposed adjacently to the second emitting part 700A. The CGL2 780 includes a second N-type CGL (N-CGL2) 785 disposed adjacently to the second emitting part 700A and a second P-type CGL (P-CGL2) 790 disposed adjacently to the third emitting part 800. Each of the N-CGL1 685 and the N-CGL2 785 injects electrons to the EML1 640 of the first emitting part 600 and the EML2 740A of the second emitting part 700A, respectively, and each of the P-CGL1 690 and the P-CGL2 790 injects holes to the EML2 740A of the second emitting part 700A and the EML3 840 of the third emitting part 800, respectively.

Each of the EML1 640 and the EML3 840 can be independently a blue EML. In this case, each of the EML1 640 and the EML3 840 can be independently a blue EML, a sky-blue EML or a deep-blue EML. Each of the EML1 640 and the EML3 840 can independently include a blue host and a blue dopant. Each of the blue host and the blue dopant can be identical to each of the blue host and the blue dopant with referring to FIG. 3. For example, the blue dopant can include at least one of the blue phosphorescent materials, the blue fluorescent materials and the blue delayed fluorescent materials. Alternatively, the blue dopant in the EML1 640 can be identical to or different from the blue dopant in the EML3 840 in terms of color and/or luminous efficiency.

The EML2 740A can include a first layer 742, a third layer 746 and a second layer 744 disposed sequentially between the EBL2 730 and the HBL2 750. One of the first layer 742 and the second layer 744 can emit red color and the other of the first layer 742 and the second layer 744 can emit green color. Hereinafter, the EML2 740A where the first layer 742 emits a red color and the second layer 744 emits a green color will be described in detail.

The first layer can include a red host and a red dopant. Each of the red host and the red dopant can be identical to each of the red host and the red dopant referring to FIG. 3. For example, the red dopant can include at least one of the red phosphorescent materials, the red fluorescent materials and the red delayed fluorescent materials.

The second layer can include a green host and a green dopant. Each of the green host and the green dopant can be identical to each of the green host and the green dopant referring to FIG. 3. For example, the green dopant can include at least one of the green phosphorescent materials, the green fluorescent materials and the green delayed fluorescent materials.

The third layer 746 can be a yellow-green emitting material layer. The third layer 746 can include a yellow-green host and a yellow-green dopant. As an example, the yellow-green host can include the red host and/or the green host. The yellow-green dopant can include at least one of yellow-green phosphorescent materials, yellow-green fluorescent materials and yellow-green delayed fluorescent materials.

As an example, the yellow-green dopant can include, but is not limited to, 5,6,11,12-Tetraphenylnaphthalene (Rubrene), 2,8-Di-tert-butyl-5,11-bis(4-tert-butylphenyl)-6,12-diphenyltetracene (TBRb), Bis(2-phenylbenzothiazolato)(acetylacetonate)iridium(III) (Ir(BT)₂(acac)), Bis(2-(9,9-diethyl-fluoren-2-yl)-1-phenyl-1H-benzo [d]imdiazolato)(acetylacetonate)iridium(III) (Ir(fbi)₂(acac)), Bis(2-phenylpyridine)(3-(pyridine-2-yl)-2H-chromen-2-onate)iridium(III) (fac-Ir(ppy)₂Pc), Bis(2-(2,4-difluorophenyl)quinoline)(picolinate)iridium(III) (FPQlrpic), Bis(4-phenylthieno [3,2-c]pyridinato-N,C_{2′}) (acetylacetonate) iridium(III) (PO-01) and/or combinations thereof. The third layer 746 can be omitted.

### Example 1 (Ex.1)

### Fabrication of OLED

An organic light emitting diode where a first electron transport layer including Compound 1-1 of Chemical Formula 3 of a spiro-fluorenyl substituted benzimidazole-based organic

Compound and a second electron transport layer including Compound 2-1 of Chemical Formula 6 of an anthracenyl substituted benzimidazole-based organic Compound are disposed between an emitting material layer and a cathode. A glass substrate onto which ITO (100 nm) was coated as a thin film was washed and ultrasonically cleaned by solvent such as isopropyl alcohol, acetone and dried at 100° C. oven. The substrate was transferred to a vacuum chamber for depositing emissive layer. Subsequently, an emissive layer and a cathode were deposited by evaporation from a heating boat under about 5-7×10⁻⁷ Torr as the following order:

A hole injection layer (HIL) (DNTPD (80 wt %), P-type dopant (20 wt %), 20 nm thickness); a hole transport layer (HTL) (DNTPD, 100 nm thickness); an electron blocking layer (EBL) (TCTA, 5 nm thickness); an emitting material layer (EML) (MADN (95 wt %), DABNA-1 (5 wt %), 20 nm thickness); a first electron transport layer (ETL1) (Compound of 1-1 of Chemical Formula 3, 10 nm thickness); a second electron transport layer (ETL2) (Compound of 2-1 of Chemical Formula 6, 10 nm thickness); an electron injection layer (EIL) (LiF, 1.5 nm thickness); and a cathode (Al, 100 nm thickness).

The structures of materials used in the HIL, HTL, EBL and the host and the dopant in the EML among the compounds used for preparing the OLED are illustrated in the following:

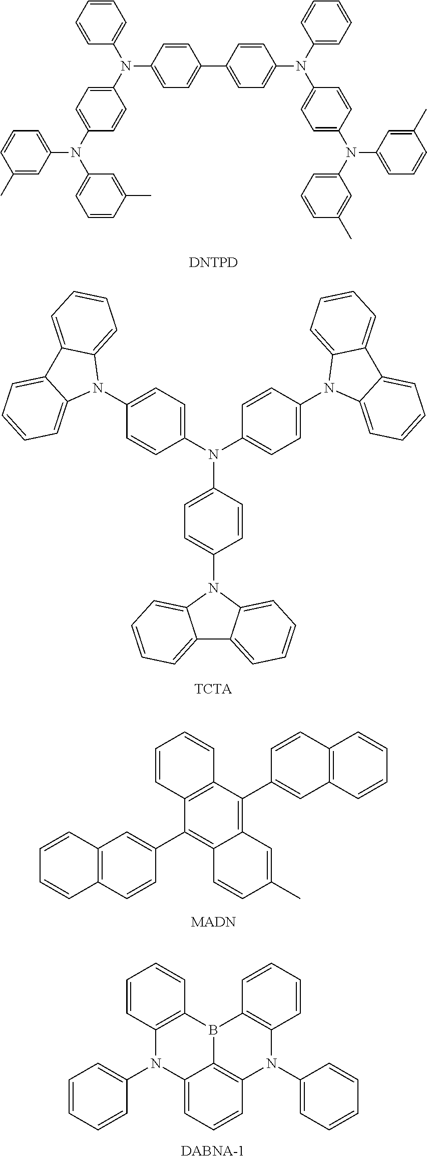

### Examples 2-9 (Ex. 2-9)

### Fabrication of OLEDs

An OLED was fabricated using the same procedure and the same material as Example 1, except that the organic compounds of the first and second electron transport layers are changed as illustrated in Table 1 below.

### Comparative Examples 1-3 (Ref. 1-3)

### Fabrication of OLED

An OLED was fabricated using the same procedure and the same material as Example 1, except that the electron transport layers are changed to a single layer (20 nm thickness) as illustrated in Table 2 below.

### Comparative Example 4-12 (Ref. 4-12)

### Fabrication of OLED

An OLED was fabricated using the same procedure and the same material as Example 1, except that the organic compounds of the first and second electron transport layer are changed as illustrated in Table 2 below.

### Experimental Example 1

### Estimation of High Temperature Stability of OLEDs

The high temperature stability of each of the OLEDs, fabricated in Examples 1 to 9 and Comparative Examples 1 to 12 was estimated by measuring blue intensity (B.I.ₒₕᵣ) of each OLED. Each of the OLEDs was thermally treated in an oven setting to a temperature of 130° C. for 1 hour, and then the blue intensity ((B.I.₁ₕᵣ) of each OLED were measured. Tables 1 and 2 below indicate measurement results of reduction rate of B.I.₁ₕᵣ relative to the B.I.ₒₕᵣ for each OLED.

TABLE 1
High Temperature Stability of OLED

| | ETL | | B Reduction |
|---|---|---|---|
| Sample | ETL1 | ETL2 | Rate* |
| Ex. 1 | Compound 1-1 | Compound 2-1 | 0.4% |
| Ex. 2 | Compound 1-1 | Compound 2-2 | 0.3% |
| Ex. 3 | Compound 1-1 | Compound 2-3 | 0.7% |
| Ex. 4 | Compound 1-2 | Compound 2-1 | 0.6% |
| Ex. 5 | Compound 1-2 | Compound 2-2 | 0.4% |
| Ex. 6 | Compound 1-2 | Compound 2-3 | 0.1% |
| Ex. 7 | Compound 1-3 | Compound 2-1 | 0.4% |
| Ex. 8 | Compound 1-3 | Compound 2-2 | 0.6% |
| Ex. 9 | Compound 1-3 | Compound 2-3 | 0.5% |

*1-B.I._{1 hr}/B.I._{0 hr}

TABLE 2
High Temperature Stability of OLED

| | ETL | | B Reduction |
|---|---|---|---|
| Sample | ETL1 | ETL2 | Rate* |
| Ref. 1 | Compound 2-1 | | 15.6% |
| Ref. 2 | Compound 2-2 | | 16.3% |
| Ref. 3 | Compound 2-3 | | 12.2% |
| Ref. 4 | Compound 2-1 | Compound 1-1 | 11.8% |
| Ref. 5 | Compound 2-1 | Compound 1-2 | 13.6% |
| Ref. 6 | Compound 2-1 | Compound 1-3 | 14.1% |
| Ref. 7 | Compound 2-2 | Compound 1-1 | 12.2% |
| Ref. 8 | Compound 2-2 | Compound 1-2 | 13.1% |
| Ref. 9 | Compound 2-2 | Compound 1-3 | 14.9% |
| Ref. 10 | Compound 2-3 | Compound 1-1 | 12.3% |
| Ref. 11 | Compound 2-3 | Compound 1-2 | 14.2% |
| Ref. 12 | Compound 2-3 | Compound 1-3 | 13.3% |

*1-B.I._{1 hr}/B.I._{0 hr}

As indicated in Tables 1 and 2, the OLEDs fabricated in Ref. 1 to Ref. 12 where the anthracene-substituted benzimidazole-based organic compounds are applied into a single-layered electron transport layer, or the anthracene-based benzimidazole-based organic compounds are applied into an ETL¹ on the EML showed 12-16% of blue light reduction rate in the high temperature stability tests. On the contrary, the OLEDs fabricated in Ex. 1 to Ex. 9 where the ETL1 including the spiro-fluorenyl substituted benzimidazole-based organic compounds are formed on the EML and the ETL2 including the anthracenyl-substituted benzimidazole-based organic compounds are formed on the ETL1 maintained their blue light intensity in the high temperature stability tests, and ensured their high temperature reliability.

### Example 10 (Ex.10)

### Fabrication of Tandem-Structured OLED

An organic light emitting diode where a first electron transport layer including Compound 1-1 of Chemical Formula 3 of a spiro-fluorenyl substituted benzimidazole-based organic Compound and a second electron transport layer including Compound 2-1 of Chemical Formula 6 of an anthracenyl substituted benzimidazole-based organic Compound are disposed between an emitting material layer of a third emitting part and a cathode. A glass substrate onto which ITO (100 nm) was coated as a thin film was washed and ultrasonically cleaned by solvent such as isopropyl alcohol, acetone and dried at 100° C. oven. The substrate was transferred to a vacuum chamber for depositing emissive layer. Subsequently, an emissive layer and a cathode were deposited by evaporation from a heating boat under about 5-7×10⁻⁷ Torr as the following order:

A hole injection layer (HIL) (MF₂, 5 nm thickness); a lower hole transport layer (B-HTL) (DNTPD, 100 nm thickness); a lower electron blocking layer (B-EBL) (TCTA, 5 nm thickness); a lower blue emitting material layer (B-EML) (MADN (95 wt %), DABNA-1 (5 wt %), 20 nm thickness); a lower electron transport layer (B-ETL) (ZADN, 5 nm thickness); a first N-type charge generation layer (N-CGL1) (Bphen (98 wt %), Li (2 wt %), 15 nm thickness); a first P-type charge generation layer (P-CGL1) (DNTPD (80 wt %), P-type dopant (20 wt %), 7 nm thickness); a middle hole transport layer (M-HTL) (BPBPA, 20 nm thickness); a red emitting material layer (R-EML) (host (BPBPA: TPBi=5:5 by weight, 95 wt %), Ir(piq)₂(acac) (5 wt %), 10 nm thickness); a yellow-green emitting material layer (YG-EML) (host (CBP: TPBi =5: 5 by weight, 85 wt %), PO-01 (15 wt %), 10 nm thickness); a green emitting material layer (G-EML) (host (CBP: TPBi=5: 5 by weight, 85 wt %), Ir(ppy)₃ (15 wt %), 20 nm thickness); a middle electron transport layer (M-ETL) (TPBi, 20 wt %); a second N-type charge generation layer (N-CGL2) (Bphen (97 wt %), Li (3 wt %), 20 nm thickness); a second P-type charge generation layer (P-CGL2) (DNTPD (80 wt %), P-type dopant (20 wt %), 10 nm thickness); an upper hole transport layer (U-HTL) (DNTPD, 100 nm thickness); an upper electron blocking layer (U-EBL) (TCTA, 5 nm thickness); an upper blue emitting material layer (U-EML) (MADN (95 wt %), DABNA-1 (5 wt %), 20 nm thickness); a first upper electron transport layer (ETL1) (Compound 1-1 of Chemical Formula 3, 10 nm thickness); a second upper electron transport layer (ETL2) (Compound 2-1 of Chemical Formula 6, 10 nm thickness); an electron injection layer (EIL) (LiF, 1.5 nm thickness); and a cathode (Al, 100 nm thickness).

The structures of materials other than materials indicated in Ex.1 to Ex. 9 among the compounds used for preparing the OLED are illustrated in the following:

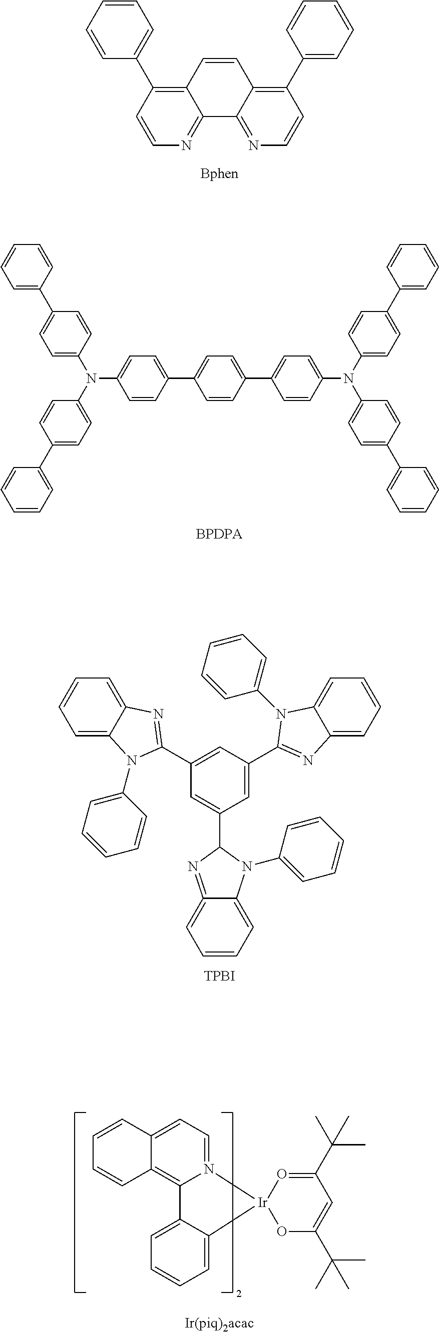

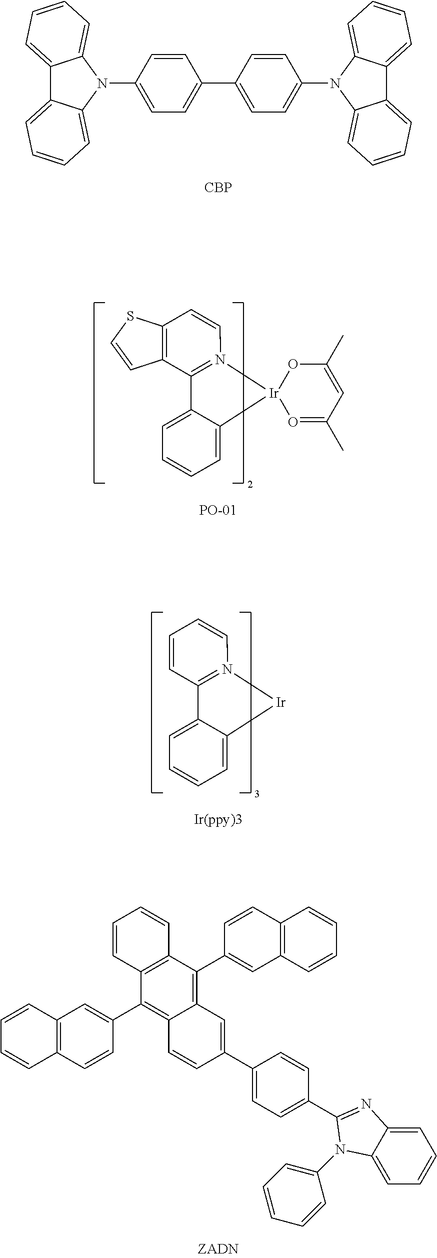

### Examples 11-18 (Ex. 11-12)

### Fabrication of OLEDs

An OLED was fabricated using the same procedure and the same material as Example 10, except that the organic compounds of the first and second electron transport layers in the third emitting part are changed as illustrated in Table 3 below.

### Comparative Example 13-21 (Ref. 13-21)

### Fabrication of OLED

An OLED was fabricated using the same procedure and the same material as Example 1, except that the organic compounds of the first and second electron transport layer in the third emitting part are changed as illustrated in Table 4 below.

### Experimental Example 2

### Estimation of High Temperature Stability of OLEDs

The high temperature stability of each of the OLEDs, fabricated in Examples 10 to 18 and Comparative Examples 13 to 21 was estimated as the Experimental Example 1 by measuring blue intensity (B.I.₀ₕᵣ), red intensity (R.I.₀ₕᵣ) and green intensity (G.I.₀ₕᵣ) of each OLED. Each of the OLEDs was thermally treated in an oven setting to a temperature of 130° C. for 1 hour, and then the blue intensity (B.I.₁ₕᵣ), red intensity(R.I.₁ₕᵣ) and green intensity (G.I.₁ₕᵣ) of each OLED were measured. Tables 3 and 4 below indicate measurement results of reduction rates of B.I.₁ₕᵣ relative to the B.I.₀ₕᵣ, R.I.₁ₕᵣ relative to the R.I.₀ₕᵣ, and G.I.₁ₕᵣ relative to the G.I.₀ₕᵣ for each OLED.

TABLE 3
High Temperature Stability of OLED

| | ETL | | B Reduction | R Reduction | G Reduction |
|---|---|---|---|---|---|
| Sample | ETL1 | ETL2 | Rate* | Rate** | Rate*** |
| Ex. 10 | Compound 1-1 | Compound 2-1 | 0.1% | 0.0% | 0.1% |
| Ex. 11 | Compound 1-1 | Compound 2-2 | 0.5% | 0.0% | 0.3% |
| Ex. 12 | Compound 1-1 | Compound 2-3 | 0.1% | 0.0% | 0.2% |
| Ex. 13 | Compound 1-2 | Compound 2-1 | 0.2% | 0.1% | 0.2% |
| Ex. 14 | Compound 1-2 | Compound 2-2 | 0.3% | 0.2% | 0.3% |
| Ex. 15 | Compound 1-2 | Compound 2-3 | 0.1% | 0.1% | 0.2% |
| Ex. 16 | Compound 1-3 | Compound 2-1 | 0.3% | 0.2% | 0.1% |
| Ex. 17 | Compound 1-3 | Compound 2-2 | 0.2% | 0.3% | 0.0% |
| Ex. 18 | Compound 1-3 | Compound 2-3 | 0.0% | 0.1% | 0.3% |

*1-B.I._{1 hr}/B.I._{0 hr};
**1-R.I._{1 hr}/R.I._{0 hr};
***1-G.I._{1 hr}/G.I._{0 hr}

TABLE 4
High Temperature Stability of OLED

| | ETL | | B Reduction | R Reduction | G Reduction |
|---|---|---|---|---|---|
| Sample | ETL1 | ETL2 | Rate* | Rate** | Rate*** |
| Ref. 13 | Compound 2-1 | Compound 1-1 | 11.1% | 0.1% | 0.2% |
| Ref. 14 | Compound 2-1 | Compound 1-2 | 12.6% | 0.0% | 0.2% |
| Ref. 15 | Compound 2-1 | Compound 1-3 | 13.2% | 0.3% | 0.1% |
| Ref. 16 | Compound 2-2 | Compound 1-1 | 14.2% | 0.2% | 0.1% |
| Ref. 17 | Compound 2-2 | Compound 1-2 | 13.5% | 0.0% | 0.3% |
| Ref. 18 | Compound 2-2 | Compound 1-3 | 13.7% | 0.1% | 0.2% |
| Ref. 19 | Compound 2-3 | Compound 1-1 | 12.9% | 0.0% | 0.1% |
| Ref. 20 | Compound 2-3 | Compound 1-2 | 11.8% | 0.1% | 0.4% |
| Ref. 21 | Compound 2-3 | Compound 1-3 | 10.9% | 0.2% | 0.0% |

*1-B.I._{1 hr}/B.I._{0 hr};
**1-R.I._{1 hr}/R.I._{0 hr};
***1-G.I._{1 hr}/G.I._{0 hr}

As indicated in Tables 3 and 4, the OLEDs fabricated in Ref. 13 to Ref. 22 where the anthracene-substituted benzimidazole-based organic compounds are applied into an ETL1 on the U-EML in the third emitting part, which were formed most adjacently to the cathode, showed 10% or more of blue light reduction rate in the high temperature stability tests. On the contrary, the OLEDs fabricated in Ex. 19 to Ex. 18 where the ETL1 including the spiro-fluorenyl substituted benzimidazole-based organic compounds are formed on the EML and the ETL2 including the anthracenyl-substituted benzimidazole-based organic compounds are formed on the ETL1 in the third emitting part maintained their blue light intensity in the high temperature stability tests.

It will be apparent to those skilled in the art that various modifications and variations may be made in the present disclosure without departing from the scope of the disclosure. Thus, it is intended that the present disclosure cover the modifications and variations of the present disclosure provided they come within the scope of the appended claims.

What is claimed is:

1. An organic light emitting diode including:
a first electrode;
a second electrode facing the first electrode; and
an emissive layer disposed between the first electrode and the second electrode, and including at least one emitting part,
wherein an emitting part located adjacently to the second electrode among the at least one emitting part includes:
at least one emitting material layer; and
an electron transport layer disposed between the at least one emitting material layer and the second electrode;
wherein the electron transport layer includes:
a first electron transport layer disposed between the at least one emitting material layer and the second electrode; and
a second electron transport layer disposed between the first electron transport layer and the second electrode,
wherein the first electron transport layer includes a first electron transporting material represented by a structure of Chemical Formula 1, and
wherein the second electron transport layer includes a second electron transporting material represented by a structure of Chemical Formula 4: 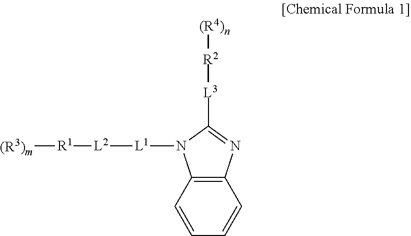
wherein, in the Chemical Formula 1,
each of R¹ and R² is independently an unsubstituted or substituted C₁-C₁₀ alkyl group, an unsubstituted or substituted C₆-C₃₀ aryl group or an unsubstituted or substituted C₃-C₃₀ hetero aryl group, and at least one of R¹ and R² is an unsubstituted or substituted spiro-fluorenyl group;
each of R³ and R⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R³ is identical to or different form each other when m is an integer of 2 or more, and where each R⁴ is identical to or different from each other when n is an integer of 2 or more;
each of L¹, L² and L³ is independently a single bond, an unsubstituted or substituted C₆-C₃₀ arylene group or an unsubstituted or substituted C₃-C₃₀ hetero arylene group; and
each of m and n is independently an integer of 0 to 7, 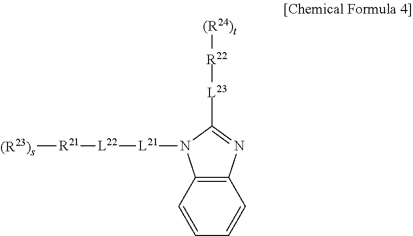
wherein, in the Chemical Formula 4,
each of R²¹ and R²² is independently an unsubstituted or substituted C₁-C₁₀ alkyl group, an unsubstituted or substituted C₆-C₃₀ aryl group or an unsubstituted or substituted C₃-C₃₀ hetero aryl group, and at least one of R²¹ and R²² is an unsubstituted or substituted anthracenyl group;
each of R²³ and R²⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R²³ is identical to or different form each other when s is an integer of 2 or more, and where each R²⁴ is identical to or different from each other when t is an integer of 2 or more;
each of L²¹, L²² and L²³ is independently a single bond, an unsubstituted or substituted C₆-C₃₀ arylene group or an unsubstituted or substituted C₃-C₃₀ hetero aryl group; and
each of s and t is independently an integer of 0 to 9.

2. The organic light emitting diode of claim 1, wherein the first electron transporting material has a structure of Chemical Formula 2: 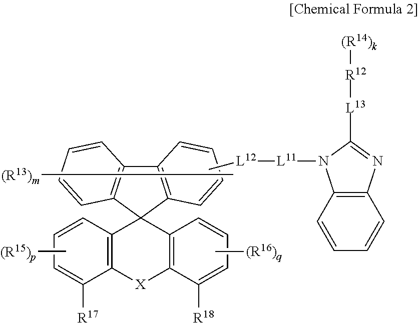
wherein, in the Chemical Formula 2,
R¹² is an unsubstituted or substituted C₁-C₁₀ alkyl group or an unsubstituted or substituted C₆-C₃₀ aryl group;
R¹³ is an unsubstituted or substituted C₁-C₁₀ alkyl group or an unsubstituted or substituted C₆-C₃₀ aryl group, and where each R¹³ is identical to or different from each other when m is an integer of 2 or more;
R¹⁴ is an unsubstituted or substituted C₁-C₁₀ alkyl group or an unsubstituted or substituted C₆-C₃₀ aryl group, and where each R¹⁴ is identical to or different from each other when k is an integer of 2 or more;
each of R¹⁵ and R¹⁶ is independently an C₁-C₁₀ alkyl group or an C₆-C₃₀ aryl group, where each R¹⁵ is identical to or different from each other when p is an integer of 2 or more, and where each R¹⁶ is identical to or different from each other when q is an integer of 2 or more;
each of R¹⁷ and R¹⁸ is independently hydrogen, an C₁-C₁₀ alkyl group or an C₆-C₃₀ aryl group, when X is a single bond, or one of R¹⁷ and R¹⁸, and X are further linked together to form an unsubstituted or substituted fused ring and the other of R¹⁷ and R¹⁸ is hydrogen, an C₁-C₁₀ alkyl group or an C₆-C₃₀ aryl group when X is nitrogen;
X is a single bond or nitrogen;
each of L¹¹, L¹² and L¹³ is independently a single bond or an unsubstituted or substituted C₆-C₃₀ arylene group;
m is a same as defined in Formula 1;
k is an integer of 0 to 3; and
each of p and q is an integer of 0 to 3.

3. The organic light emitting diode of claim 1, wherein the first electron transporting material is selected from:

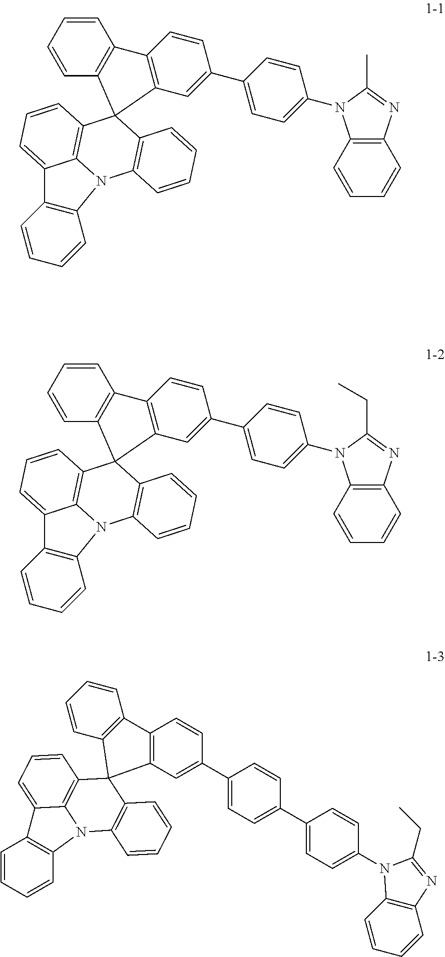 [SMILES: Cc1nc2ccccc2n1-c1ccc(-c2ccc3c(c2)C2(c4ccccc4-3)c3ccccc3-n3c4ccccc4c4cccc2c43)cc1 ; CCc1nc2ccccc2n1-c1ccc(-c2ccc3c(c2)C2(c4ccccc4-3)c3ccccc3-n3c4ccccc4c4cccc2c43)cc1 ; CCc1nc2ccccc2n1-c1ccc(-c2ccc(-c3ccc4c(c3)C3(c5ccccc5-4)c4ccccc4-n4c5ccccc5c5cccc3c54)cc2)cc1]

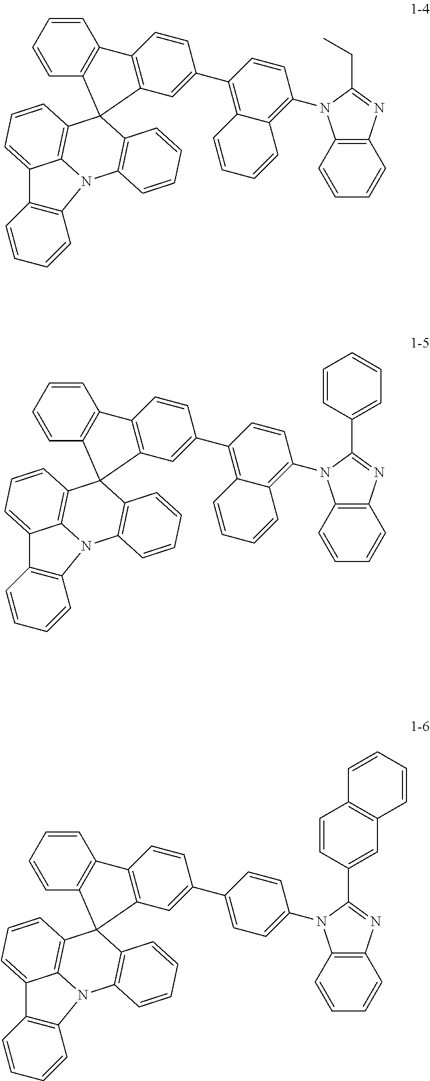 [SMILES: CCc1nc2ccccc2n1-c1ccc(-c2ccc3c(c2)C2(c4ccccc4-3)c3ccccc3-n3c4ccccc4c4cccc2c43)c2ccccc12 ; c1ccc(-c2nc3ccccc3n2-c2ccc(-c3ccc4c(c3)C3(c5ccccc5-4)c4ccccc4-n4c5ccccc5c5cccc3c54)c3ccccc23)cc1 ; c1ccc2c(c1)-c1ccc(-c3ccc(-n4c(-c5ccc6ccccc6c5)nc5ccccc54)cc3)cc1C21c2ccccc2-n2c3ccccc3c3cccc1c32]

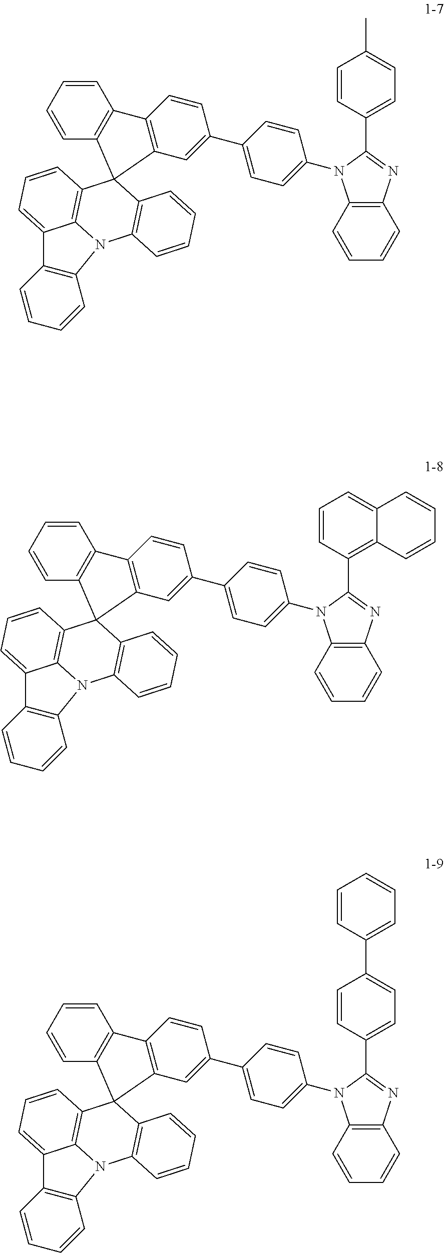 [SMILES: Cc1ccc(-c2nc3ccccc3n2-c2ccc(-c3ccc4c(c3)C3(c5ccccc5-4)c4ccccc4-n4c5ccccc5c5cccc3c54)cc2)cc1 ; c1ccc2c(c1)-c1ccc(-c3ccc(-n4c(-c5cccc6ccccc56)nc5ccccc54)cc3)cc1C21c2ccccc2-n2c3ccccc3c3cccc1c32 ; c1ccc(-c2ccc(-c3nc4ccccc4n3-c3ccc(-c4ccc5c(c4)C4(c6ccccc6-5)c5ccccc5-n5c6ccccc6c6cccc4c65)cc3)cc2)cc1]

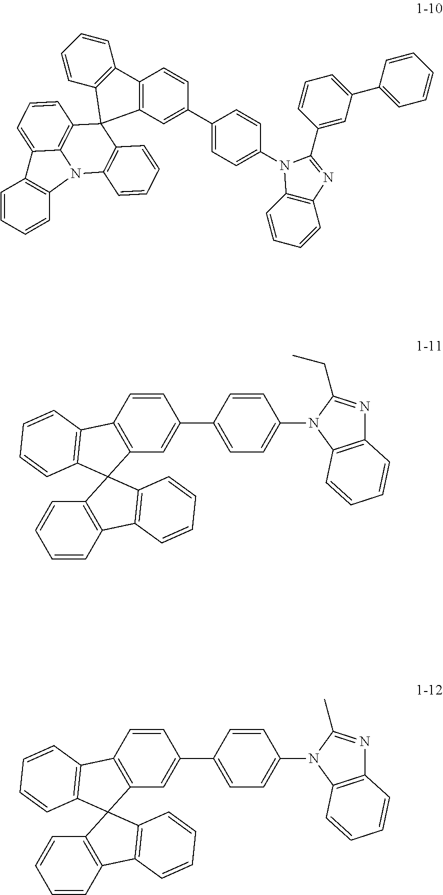 [SMILES: c1ccc(-c2cccc(-c3nc4ccccc4n3-c3ccc(-c4ccc5c(c4)C4(c6ccccc6-5)c5ccccc5-n5c6ccccc6c6cccc4c65)cc3)c2)cc1 ; CCc1nc2ccccc2n1-c1ccc(-c2ccc3c(c2)C2(c4ccccc4-c4ccccc42)c2ccccc2-3)cc1 ; Cc1nc2ccccc2n1-c1ccc(-c2ccc3c(c2)C2(c4ccccc4-c4ccccc42)c2ccccc2-3)cc1]

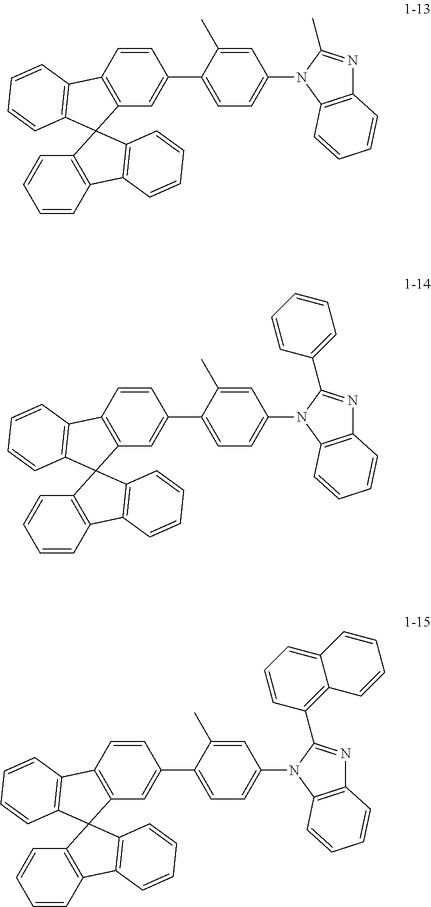 [SMILES: Cc1cc(-n2c(C)nc3ccccc32)ccc1-c1ccc2c(c1)C1(c3ccccc3-c3ccccc31)c1ccccc1-2 ; Cc1cc(-n2c(-c3ccccc3)nc3ccccc32)ccc1-c1ccc2c(c1)C1(c3ccccc3-c3ccccc31)c1ccccc1-2 ; Cc1cc(-n2c(-c3cccc4ccccc34)nc3ccccc32)ccc1-c1ccc2c(c1)C1(c3ccccc3-c3ccccc31)c1ccccc1-2]

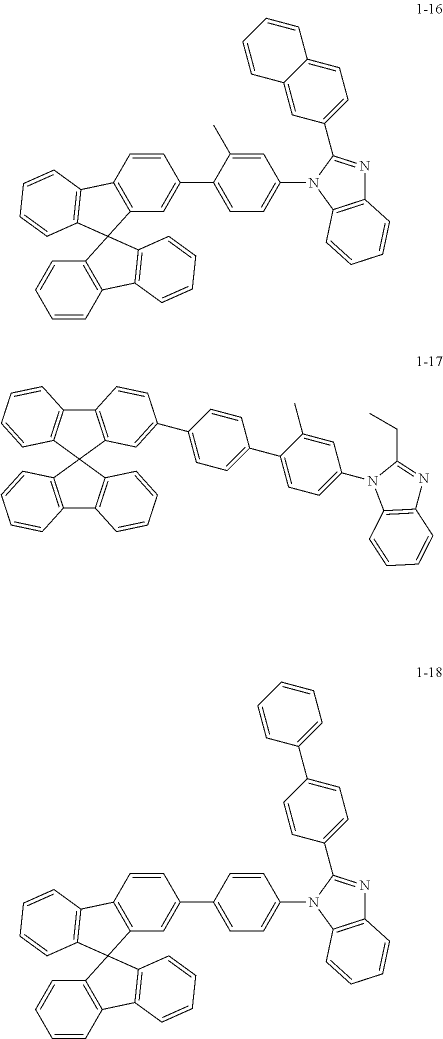 [SMILES: Cc1cc(-n2c(-c3ccc4ccccc4c3)nc3ccccc32)ccc1-c1ccc2c(c1)C1(c3ccccc3-c3ccccc31)c1ccccc1-2 ; CCc1nc2ccccc2n1-c1ccc(-c2ccc(-c3ccc4c(c3)C3(c5ccccc5-c5ccccc53)c3ccccc3-4)cc2)c(C)c1 ; c1ccc(-c2ccc(-c3nc4ccccc4n3-c3ccc(-c4ccc5c(c4)C4(c6ccccc6-c6ccccc64)c4ccccc4-5)cc3)cc2)cc1]

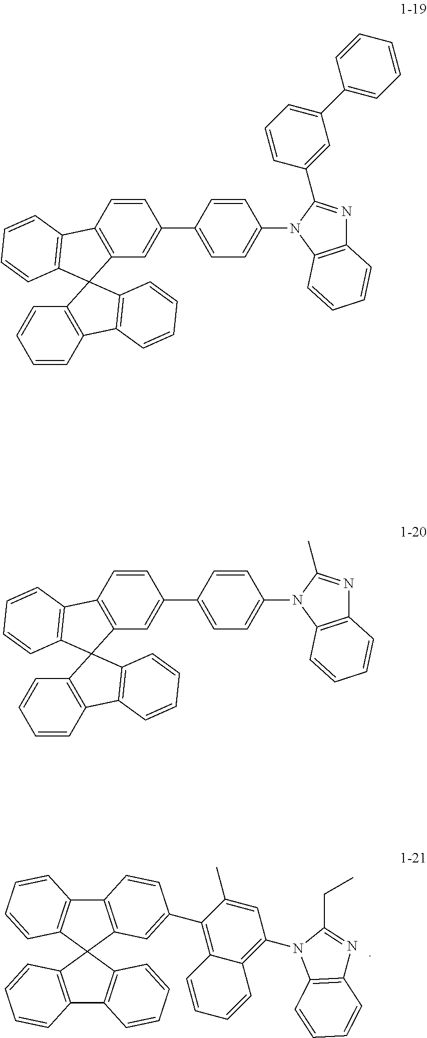 [SMILES: c1ccc(-c2cccc(-c3nc4ccccc4n3-c3ccc(-c4ccc5c(c4)C4(c6ccccc6-c6ccccc64)c4ccccc4-5)cc3)c2)cc1 ; Cc1nc2ccccc2n1-c1ccc(-c2ccc3c(c2)C2(c4ccccc4-c4ccccc42)c2ccccc2-3)cc1 ; CCc1nc2ccccc2n1-c1cc(C)c(-c2ccc3c(c2)C2(c4ccccc4-c4ccccc42)c2ccccc2-3)c2ccccc12]

4. The organic light emitting diode of claim 1, wherein the second electron transporting material has a structure of Chemical Formula 5: 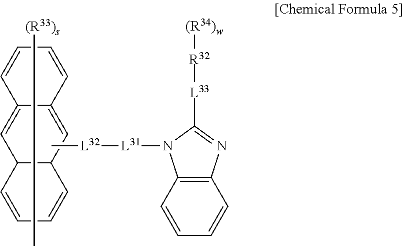
wherein, in the Chemical Formula 5,
R³³ is an unsubstituted or substituted C₁-C₁₀ alkyl group or an unsubstituted or substituted C₆-C₃₀ aryl group;
R³³ is an unsubstituted or substituted C₁-C₁₀ alkyl group or an unsubstituted or substituted C₆-C₃₀ aryl group, and where each R³³ is identical to or different from each other when s is an integer of 2 or more;
R³⁴ is an unsubstituted or substituted C₆-C₃₀ aryl group, and where each R³⁴ is identical to or different from each other when w is an integer of 2 or more;
each of L³¹, L³² and L³³ is independently a single bond or an unsubstituted or substituted C₆-C₃₀ arylene group;
s is a same as defined in Formula 4; and
w is 0 or 1.

5. The organic light emitting diode of claim 1, wherein the second electron transporting material is selected from:

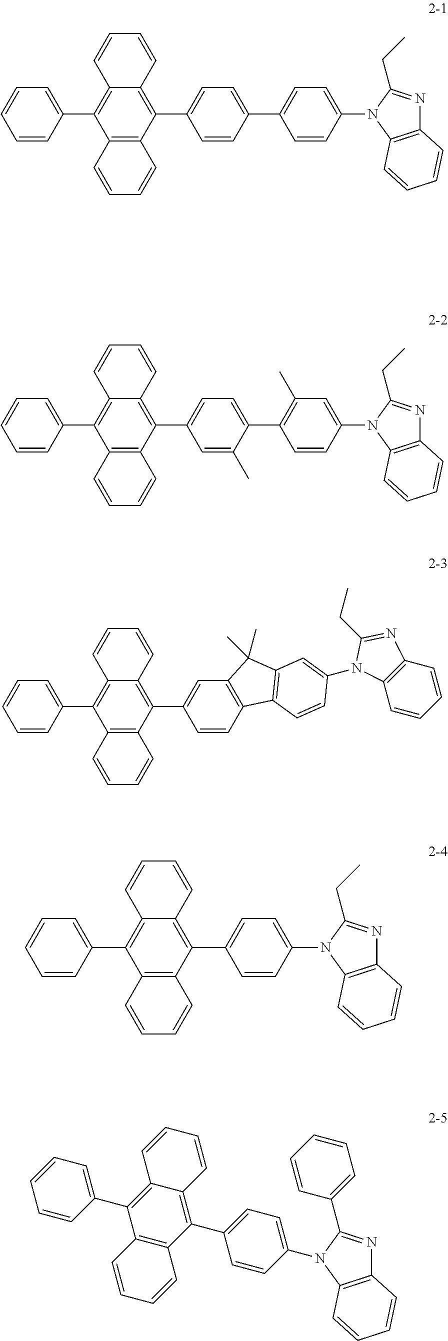 [SMILES: CCc1nc2ccccc2n1-c1ccc(-c2ccc(-c3c4ccccc4c(-c4ccccc4)c4ccccc34)cc2)cc1 ; CCc1nc2ccccc2n1-c1ccc(-c2ccc(-c3c4ccccc4c(-c4ccccc4)c4ccccc34)cc2C)c(C)c1 ; CCc1nc2ccccc2n1-c1ccc2c(c1)C(C)(C)c1cc(-c3c4ccccc4c(-c4ccccc4)c4ccccc34)ccc1-2 ; CCc1nc2ccccc2n1-c1ccc(-c2c3ccccc3c(-c3ccccc3)c3ccccc23)cc1 ; c1ccc(-c2c3ccccc3c(-c3ccc(-n4c(-c5ccccc5)nc5ccccc54)cc3)c3ccccc23)cc1]

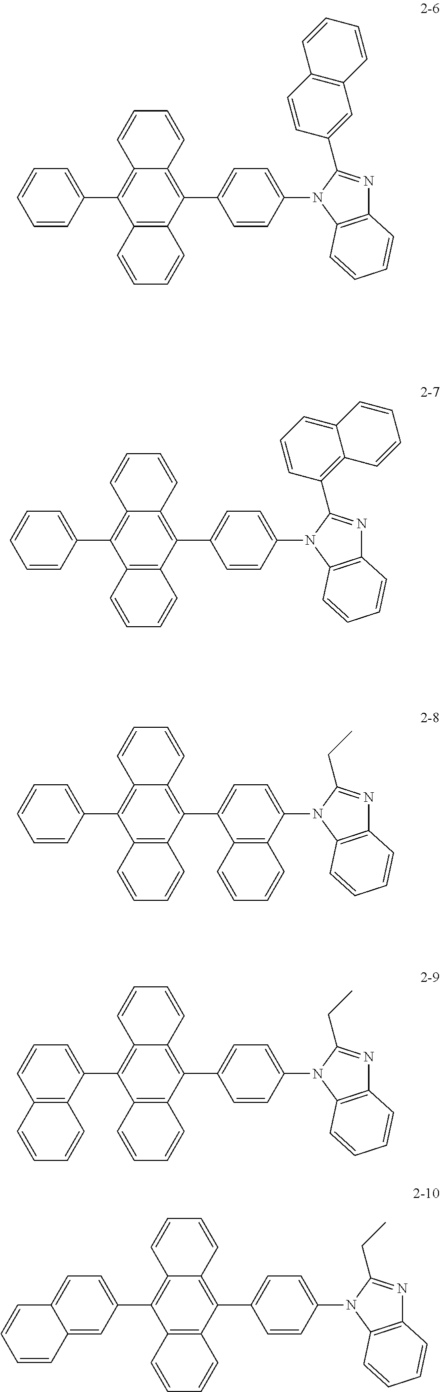 [SMILES: c1ccc(-c2c3ccccc3c(-c3ccc(-n4c(-c5ccc6ccccc6c5)nc5ccccc54)cc3)c3ccccc23)cc1 ; c1ccc(-c2c3ccccc3c(-c3ccc(-n4c(-c5cccc6ccccc56)nc5ccccc54)cc3)c3ccccc23)cc1 ; CCc1nc2ccccc2n1-c1ccc(-c2c3ccccc3c(-c3ccccc3)c3ccccc23)c2ccccc12 ; CCc1nc2ccccc2n1-c1ccc(-c2c3ccccc3c(-c3cccc4ccccc34)c3ccccc23)cc1 ; CCc1nc2ccccc2n1-c1ccc(-c2c3ccccc3c(-c3ccc4ccccc4c3)c3ccccc23)cc1]

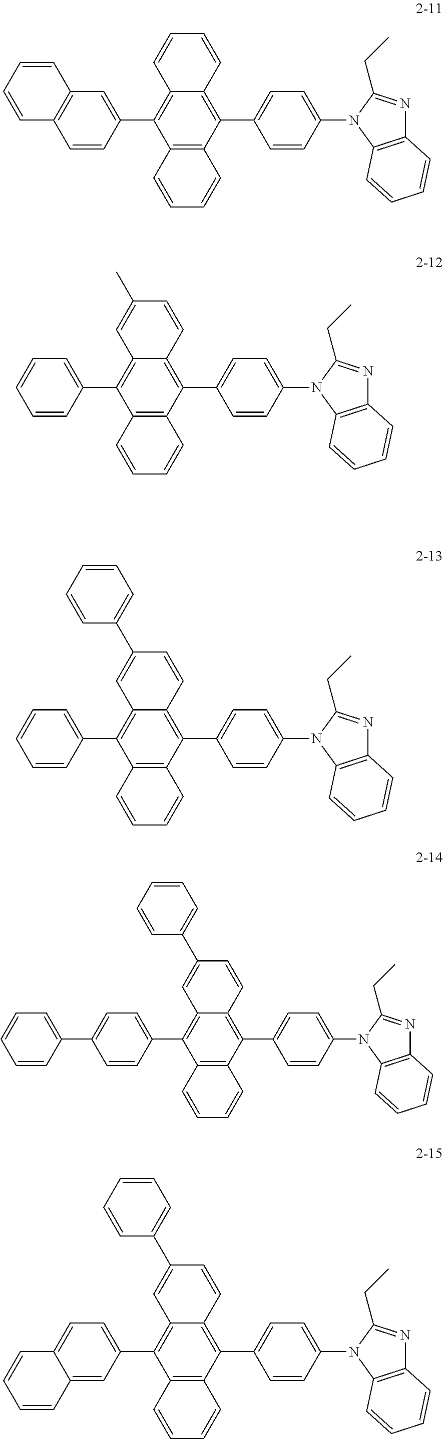 [SMILES: CCc1nc2ccccc2n1-c1ccc(-c2c3ccccc3c(-c3ccc4ccccc4c3)c3ccccc23)cc1 ; CCc1nc2ccccc2n1-c1ccc(-c2c3ccccc3c(-c3ccccc3)c3cc(C)ccc23)cc1 ; CCc1nc2ccccc2n1-c1ccc(-c2c3ccccc3c(-c3ccccc3)c3cc(-c4ccccc4)ccc23)cc1 ; CCc1nc2ccccc2n1-c1ccc(-c2c3ccccc3c(-c3ccc(-c4ccccc4)cc3)c3cc(-c4ccccc4)ccc23)cc1 ; CCc1nc2ccccc2n1-c1ccc(-c2c3ccccc3c(-c3ccc4ccccc4c3)c3cc(-c4ccccc4)ccc23)cc1]

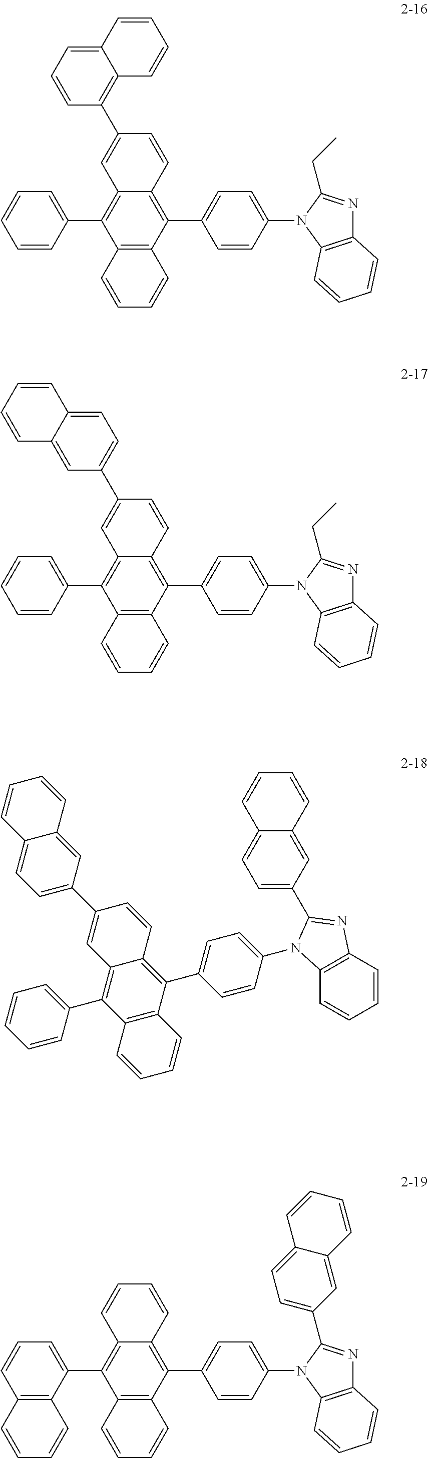 [SMILES: CCc1nc2ccccc2n1-c1ccc(-c2c3ccccc3c(-c3ccccc3)c3cc(-c4cccc5ccccc45)ccc23)cc1 ; CCc1nc2ccccc2n1-c1ccc(-c2c3ccccc3c(-c3ccccc3)c3cc(-c4ccc5ccccc5c4)ccc23)cc1 ; c1ccc(-c2c3ccccc3c(-c3ccc(-n4c(-c5ccc6ccccc6c5)nc5ccccc54)cc3)c3ccc(-c4ccc5ccccc5c4)cc23)cc1 ; c1ccc2cc(-c3nc4ccccc4n3-c3ccc(-c4c5ccccc5c(-c5cccc6ccccc56)c5ccccc45)cc3)ccc2c1]

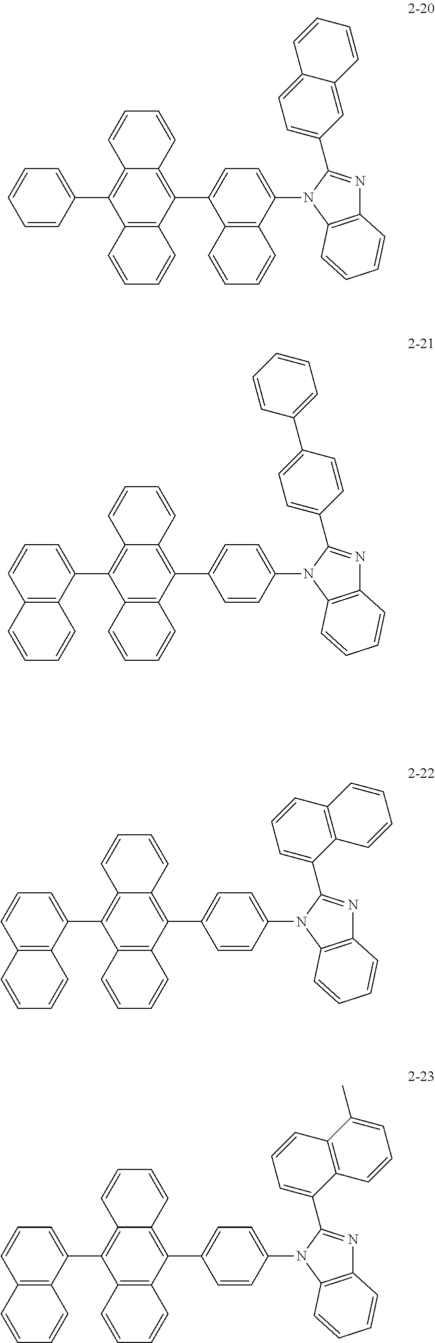 [SMILES: c1ccc(-c2c3ccccc3c(-c3ccc(-n4c(-c5ccc6ccccc6c5)nc5ccccc54)c4ccccc34)c3ccccc23)cc1 ; c1ccc(-c2ccc(-c3nc4ccccc4n3-c3ccc(-c4c5ccccc5c(-c5cccc6ccccc56)c5ccccc45)cc3)cc2)cc1 ; c1ccc2c(-c3c4ccccc4c(-c4ccc(-n5c(-c6cccc7ccccc67)nc6ccccc65)cc4)c4ccccc34)cccc2c1 ; Cc1cccc2c(-c3nc4ccccc4n3-c3ccc(-c4c5ccccc5c(-c5cccc6ccccc56)c5ccccc45)cc3)cccc12]

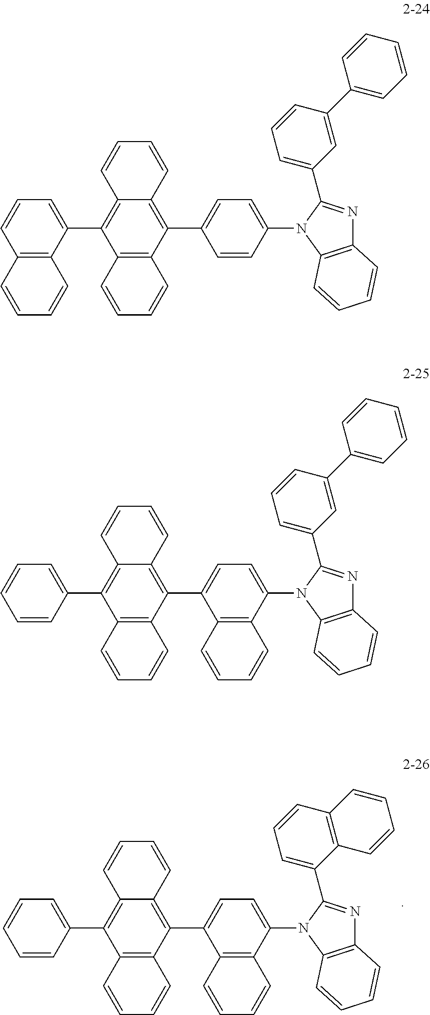 [SMILES: c1ccc(-c2cccc(-c3nc4ccccc4n3-c3ccc(-c4c5ccccc5c(-c5cccc6ccccc56)c5ccccc45)cc3)c2)cc1 ; c1ccc(-c2cccc(-c3nc4ccccc4n3-c3ccc(-c4c5ccccc5c(-c5ccccc5)c5ccccc45)c4ccccc34)c2)cc1 ; c1ccc(-c2c3ccccc3c(-c3ccc(-n4c(-c5cccc6ccccc56)nc5ccccc54)c4ccccc34)c3ccccc23)cc1]

6. The organic light emitting diode of claim 1, wherein the emissive layer has a single emitting part.

7. The organic light emitting diode of claim 1, wherein the at least one emitting material layer emits at least one of a red color, a green color, a blue color and a yellow-green color.

8. The organic light emitting diode of claim 1, wherein the emissive layer includes:
a first emitting part disposed between the first electrode and the second electrode;
a second emitting part disposed between the first emitting part and the second electrode; and
a charge generation layer disposed between the first emitting part and the second emitting part, and
wherein the second emitting part includes the at least one emitting material layer and the electron transport layer.

9. The organic light emitting diode of claim 8, wherein the at least one emitting material layer includes:
a first layer disposed between the charge generation layer and the second electrode; and
a second layer disposed between the first layer and the second electrode.

10. The organic light emitting diode of claim 9, wherein the at least one emitting material layer further includes a third layer disposed between the first layer and the second layer.

11. The organic light emitting diode of claim 1, wherein the emissive layer includes:
a first emitting part disposed between the first electrode and the second electrode;
a second emitting part disposed between the first emitting part and the second electrode;
a third emitting part disposed between the second emitting part and the second electrode;
a first charge generation layer disposed between the first emitting part and the second emitting part; and
a second charge generation layer disposed between the second emitting part and the third emitting part, and
wherein the third emitting part includes the at least one emitting material layer and the electron transport layer.

12. An organic light emitting diode including:
a first electrode;
a second electrode facing the first electrode; and
an emissive layer disposed between the first electrode and the second electrode, and including at least one emitting part,
wherein an emitting part located adjacently to the second electrode among the at least one emitting part includes:
a blue emitting material layer; and
an electron transport layer disposed between the blue emitting material layer and the second electrode,
wherein the electron transport layer includes a first electron transport layer and a second electron transport layer disposed sequentially between the at least one emitting material layer and the second electrode,
wherein the first electron transport layer includes a first electron transporting material represented by a structure of Chemical Formula 1, and
wherein the second electron transport layer includes a second electron transporting material represented by a structure of Chemical Formula 4: 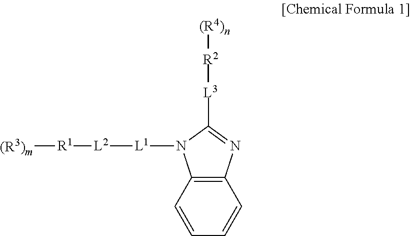
wherein, in the Chemical Formula 1,
each of R¹ and R² is independently an unsubstituted or substituted C₁-C₁₀ alkyl group, an unsubstituted or substituted C₆-C₃₀ aryl group or an unsubstituted or substituted C₃-C₃₀ hetero aryl group, and at least one of R¹ and R² is an unsubstituted or substituted spiro-fluorenyl group;
each of R³ and R⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R³ is identical to or different form each other when m is an integer of 2 or more, and where each R⁴ is identical to or different from each other when n is an integer of 2 or more;
each of L¹, L² and L³ is independently a single bond, an unsubstituted or substituted C₆-C₃₀ arylene group or an unsubstituted or substituted C₃-C₃₀ hetero arylene group; and
each of m and n is independently an integer of 0 to 7, 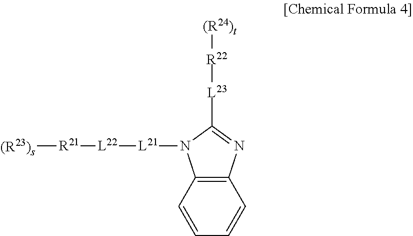
wherein, in the Chemical Formula 4,
each of R²¹ and R²² is independently an unsubstituted or substituted C₁-C₁₀ alkyl group, an unsubstituted or substituted C₆-C₃₀ aryl group or an unsubstituted or substituted C₃-C₃₀ hetero aryl group, and at least one of R²¹ and R²² is an unsubstituted or substituted anthracenyl group;
each of R²³ and R²⁴ is independently an unsubstituted or substituted C₁-C₁₀ alkyl group, an unsubstituted or substituted C₆-C₃₀ aryl group or an unsubstituted or substituted C₃-C₃₀ hetero aryl group, where each R²³ is identical to or different form each other when s is an integer of 2 or more, and where each R²⁴ is identical to or different from each other when t is an integer of 2 or more;
each of L²¹, L²² and L²³ is independently a single bond, an unsubstituted or substituted C₆-C₃₀ arylene group or an unsubstituted or substituted C₃-C₃₀ hetero arylene group; and
each of s and t is independently an integer of 0 to 9.

13. The organic light emitting diode of claim 12, wherein the first electron transporting material has a structure of Chemical Formula 2: 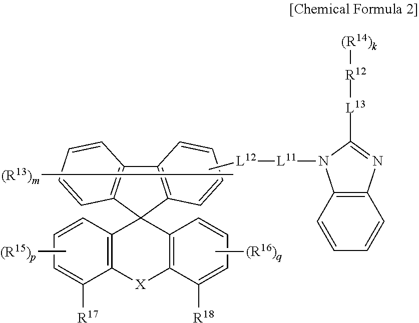
wherein, in the Chemical Formula 2,
R¹² is an unsubstituted or substituted C₁-C₁₀ alkyl group or an unsubstituted or substituted C₆-C₃₀ aryl group;
R¹³ is an unsubstituted or substituted C₁-C₁₀ alkyl group or an unsubstituted or substituted C₆-C₃₀ aryl group, and where each R¹³ is identical to or different from each other when m is an integer of 2 or more;
R¹⁴ is an unsubstituted or substituted C₁-C₁₀ alkyl group or an unsubstituted or substituted C₆-C₃₀ aryl group, and where each R¹⁴ is identical to or different from each other when k is an integer of 2 or more;
each of R¹⁵ and R¹⁶ is independently an C₁-C₁₀ alkyl group or an C₆-C₃₀ aryl group, where each R¹⁵ is identical to or different from each other when p is an integer of 2 or more, and where each R¹⁶ is identical to or different from each other when q is an integer of 2 or more;
each of R¹⁷ and R¹⁸ is independently hydrogen, an C₁-C₁₀ alkyl group or an C₆-C₃₀ aryl group, when X is a single bond, or one of R¹⁷ and R¹⁸, and X are further linked together to form an unsubstituted or substituted fused ring and the other of R¹⁷ and R¹⁸ is hydrogen, an C₁-C₁₀ alkyl group or an C₆-C₃₀ aryl group when X is nitrogen;
X is a single bond or nitrogen;
each of L¹¹, L¹² and L¹³ is independently a single bond or an unsubstituted or substituted C₆-C₃₀ arylene group;
m is a same as defined in Formula 1;
k is an integer of 0 to 3; and
each of p and q is an integer of 0 to 3.

14. The organic light emitting diode of claim 12, wherein the first electron transporting material is selected from:

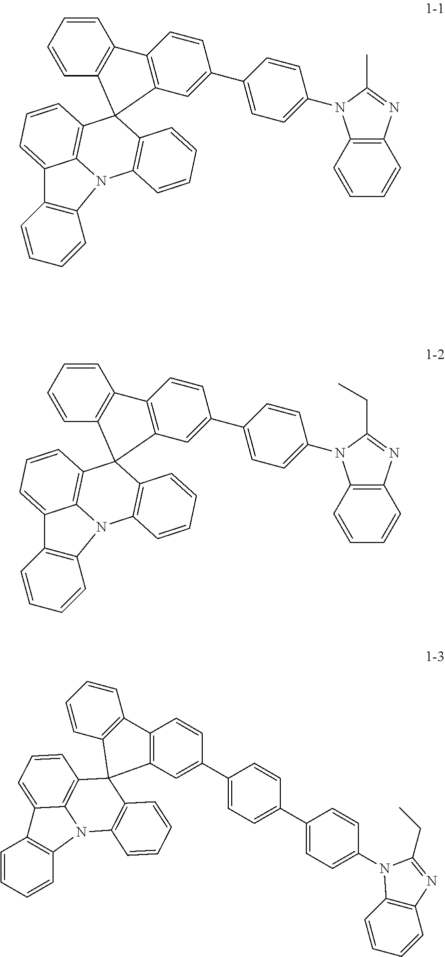 [SMILES: Cc1nc2ccccc2n1-c1ccc(-c2ccc3c(c2)C2(c4ccccc4-3)c3ccccc3-n3c4ccccc4c4cccc2c43)cc1 ; CCc1nc2ccccc2n1-c1ccc(-c2ccc3c(c2)C2(c4ccccc4-3)c3ccccc3-n3c4ccccc4c4cccc2c43)cc1 ; CCc1nc2ccccc2n1-c1ccc(-c2ccc(-c3ccc4c(c3)C3(c5ccccc5-4)c4ccccc4-n4c5ccccc5c5cccc3c54)cc2)cc1]

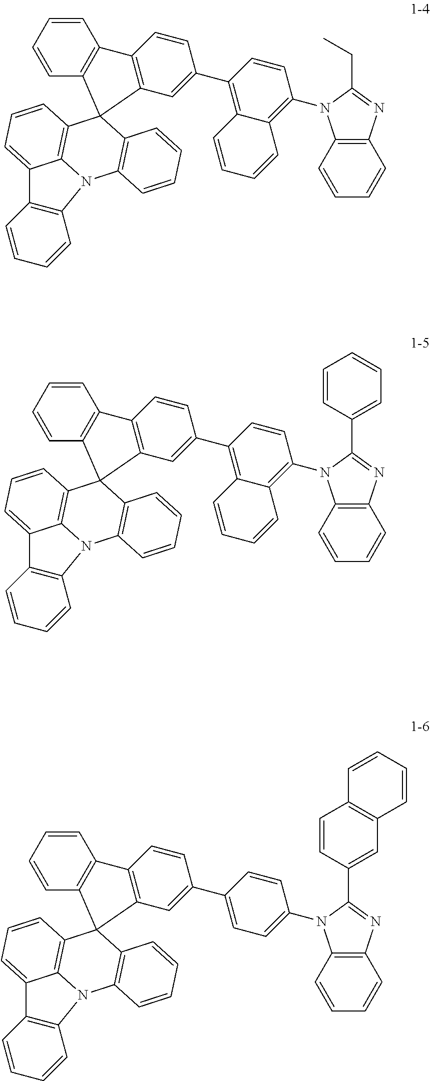 [SMILES: CCc1nc2ccccc2n1-c1ccc(-c2ccc3c(c2)C2(c4ccccc4-3)c3ccccc3-n3c4ccccc4c4cccc2c43)c2ccccc12 ; c1ccc(-c2nc3ccccc3n2-c2ccc(-c3ccc4c(c3)C3(c5ccccc5-4)c4ccccc4-n4c5ccccc5c5cccc3c54)c3ccccc23)cc1 ; c1ccc2c(c1)-c1ccc(-c3ccc(-n4c(-c5ccc6ccccc6c5)nc5ccccc54)cc3)cc1C21c2ccccc2-n2c3ccccc3c3cccc1c32]

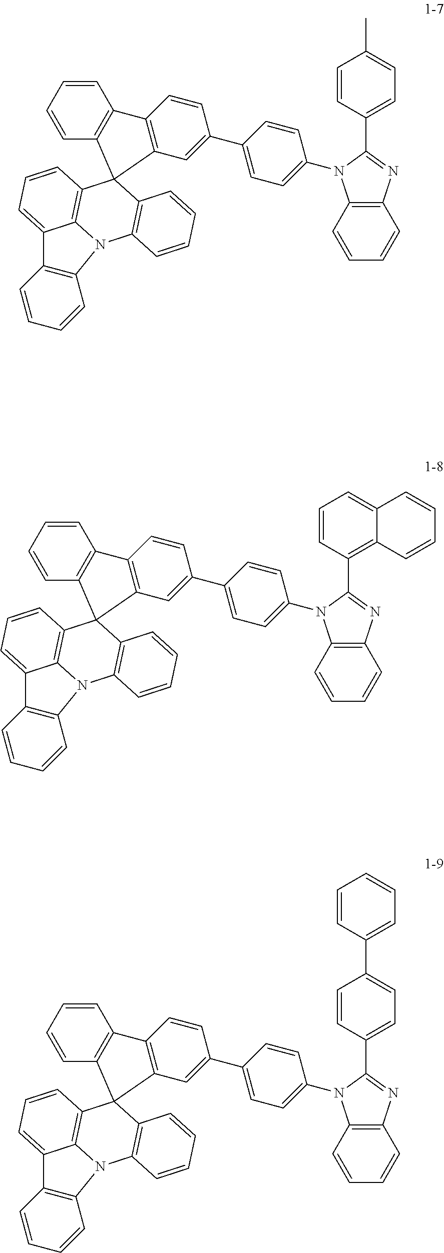 [SMILES: Cc1ccc(-c2nc3ccccc3n2-c2ccc(-c3ccc4c(c3)C3(c5ccccc5-4)c4ccccc4-n4c5ccccc5c5cccc3c54)cc2)cc1 ; c1ccc2c(c1)-c1ccc(-c3ccc(-n4c(-c5cccc6ccccc56)nc5ccccc54)cc3)cc1C21c2ccccc2-n2c3ccccc3c3cccc1c32 ; c1ccc(-c2ccc(-c3nc4ccccc4n3-c3ccc(-c4ccc5c(c4)C4(c6ccccc6-5)c5ccccc5-n5c6ccccc6c6cccc4c65)cc3)cc2)cc1]

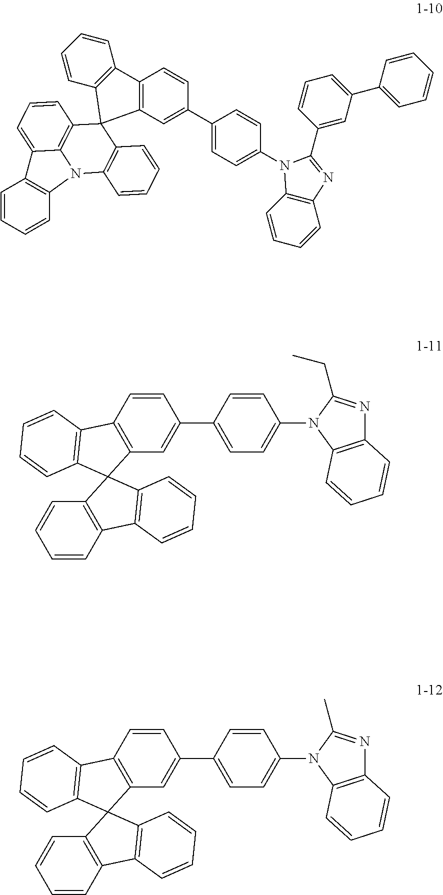 [SMILES: c1ccc(-c2cccc(-c3nc4ccccc4n3-c3ccc(-c4ccc5c(c4)C4(c6ccccc6-5)c5ccccc5-n5c6ccccc6c6cccc4c65)cc3)c2)cc1 ; CCc1nc2ccccc2n1-c1ccc(-c2ccc3c(c2)C2(c4ccccc4-c4ccccc42)c2ccccc2-3)cc1 ; Cc1nc2ccccc2n1-c1ccc(-c2ccc3c(c2)C2(c4ccccc4-c4ccccc42)c2ccccc2-3)cc1]

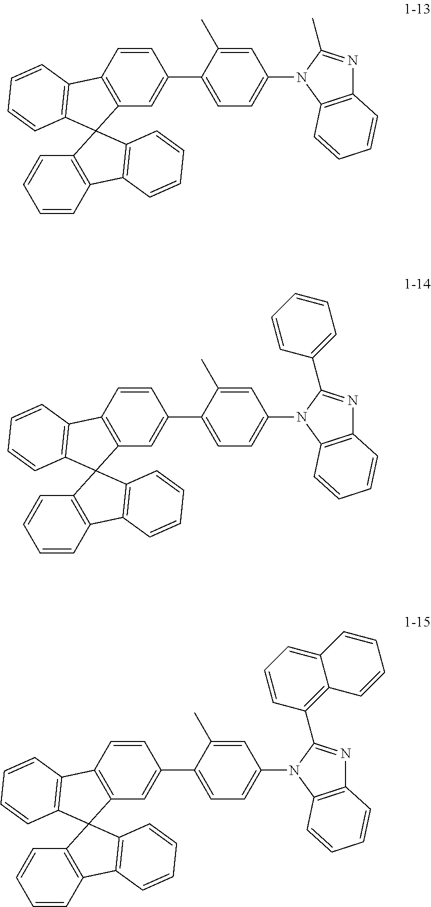 [SMILES: Cc1cc(-n2c(C)nc3ccccc32)ccc1-c1ccc2c(c1)C1(c3ccccc3-c3ccccc31)c1ccccc1-2 ; Cc1cc(-n2c(-c3ccccc3)nc3ccccc32)ccc1-c1ccc2c(c1)C1(c3ccccc3-c3ccccc31)c1ccccc1-2 ; Cc1cc(-n2c(-c3cccc4ccccc34)nc3ccccc32)ccc1-c1ccc2c(c1)C1(c3ccccc3-c3ccccc31)c1ccccc1-2]

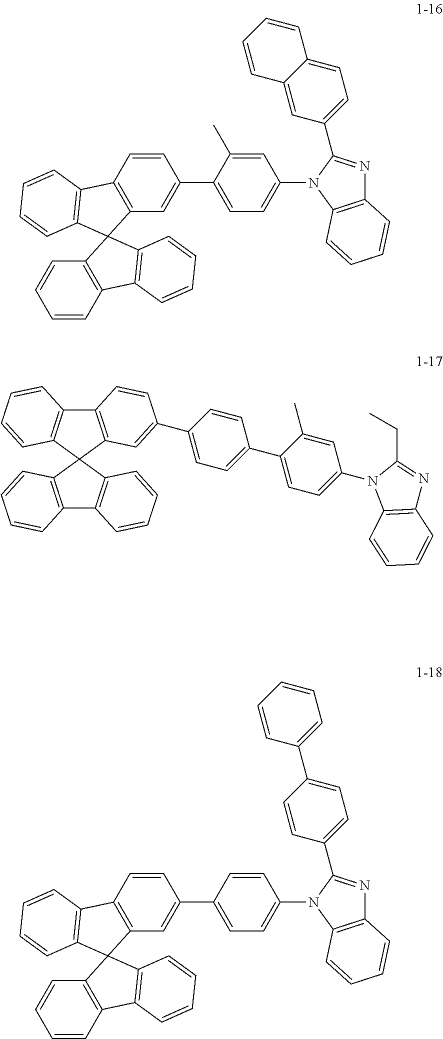 [SMILES: Cc1cc(-n2c(-c3ccc4ccccc4c3)nc3ccccc32)ccc1-c1ccc2c(c1)C1(c3ccccc3-c3ccccc31)c1ccccc1-2 ; CCc1nc2ccccc2n1-c1ccc(-c2ccc(-c3ccc4c(c3)C3(c5ccccc5-c5ccccc53)c3ccccc3-4)cc2)c(C)c1 ; c1ccc(-c2ccc(-c3nc4ccccc4n3-c3ccc(-c4ccc5c(c4)C4(c6ccccc6-c6ccccc64)c4ccccc4-5)cc3)cc2)cc1]

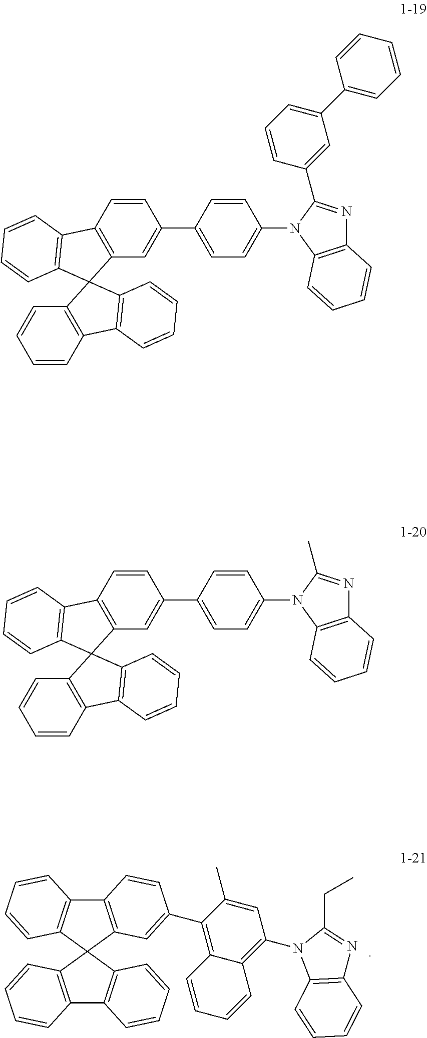 [SMILES: c1ccc(-c2cccc(-c3nc4ccccc4n3-c3ccc(-c4ccc5c(c4)C4(c6ccccc6-c6ccccc64)c4ccccc4-5)cc3)c2)cc1 ; Cc1nc2ccccc2n1-c1ccc(-c2ccc3c(c2)C2(c4ccccc4-c4ccccc42)c2ccccc2-3)cc1 ; CCc1nc2ccccc2n1-c1cc(C)c(-c2ccc3c(c2)C2(c4ccccc4-c4ccccc42)c2ccccc2-3)c2ccccc12]

15. The organic light emitting diode of claim 12, wherein the second electron transporting material has a structure of Chemical Formula 5: 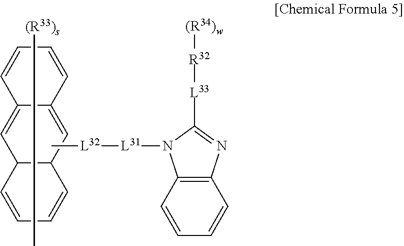
wherein, in the Chemical Formula 5,
R³² is an unsubstituted or substituted C₁-C₁₀ alkyl group or an unsubstituted or substituted C₆-C₃₀ aryl group;
R³³ is an unsubstituted or substituted C₁-C₁₀ alkyl group or an unsubstituted or substituted C₆-C₃₀ aryl group, and where each R³³ is identical to or different from each other when s is an integer of 2 or more;
R³⁴ is an unsubstituted or substituted C₆-C₃₀ aryl group, and where each R³⁴ is identical to or different from each other when w is an integer of 2 or more;
each of L³¹, L³² and L³³ is independently a single bond or an unsubstituted or substituted C₆-C₃₀ arylene group;
s is a same as defined in Formula 4; and
w is 0 or 1.

16. The organic light emitting diode of claim 12, wherein the second electron transporting material is selected from:

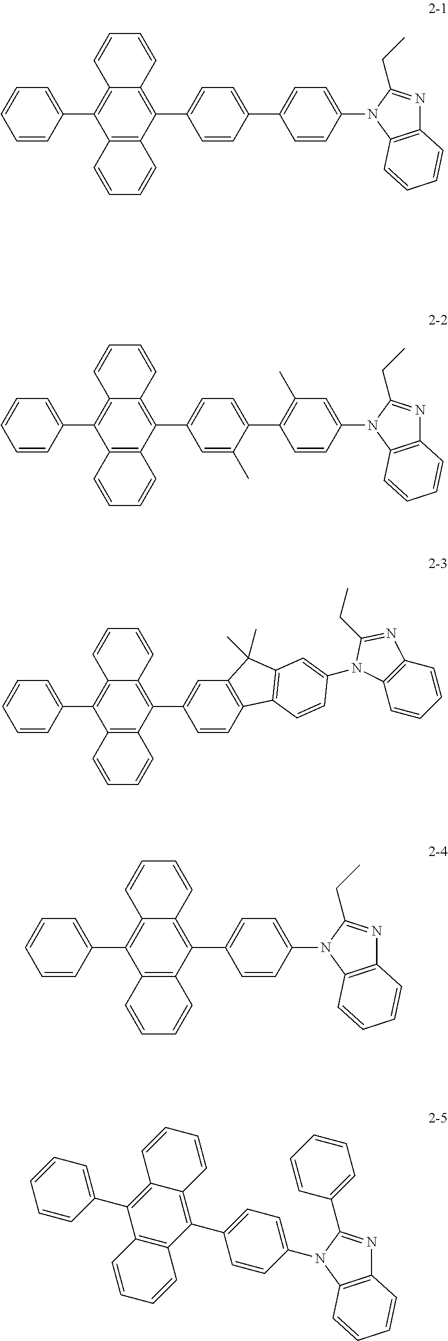 [SMILES: CCc1nc2ccccc2n1-c1ccc(-c2ccc(-c3c4ccccc4c(-c4ccccc4)c4ccccc34)cc2)cc1 ; CCc1nc2ccccc2n1-c1ccc(-c2ccc(-c3c4ccccc4c(-c4ccccc4)c4ccccc34)cc2C)c(C)c1 ; CCc1nc2ccccc2n1-c1ccc2c(c1)C(C)(C)c1cc(-c3c4ccccc4c(-c4ccccc4)c4ccccc34)ccc1-2 ; CCc1nc2ccccc2n1-c1ccc(-c2c3ccccc3c(-c3ccccc3)c3ccccc23)cc1 ; c1ccc(-c2c3ccccc3c(-c3ccc(-n4c(-c5ccccc5)nc5ccccc54)cc3)c3ccccc23)cc1]

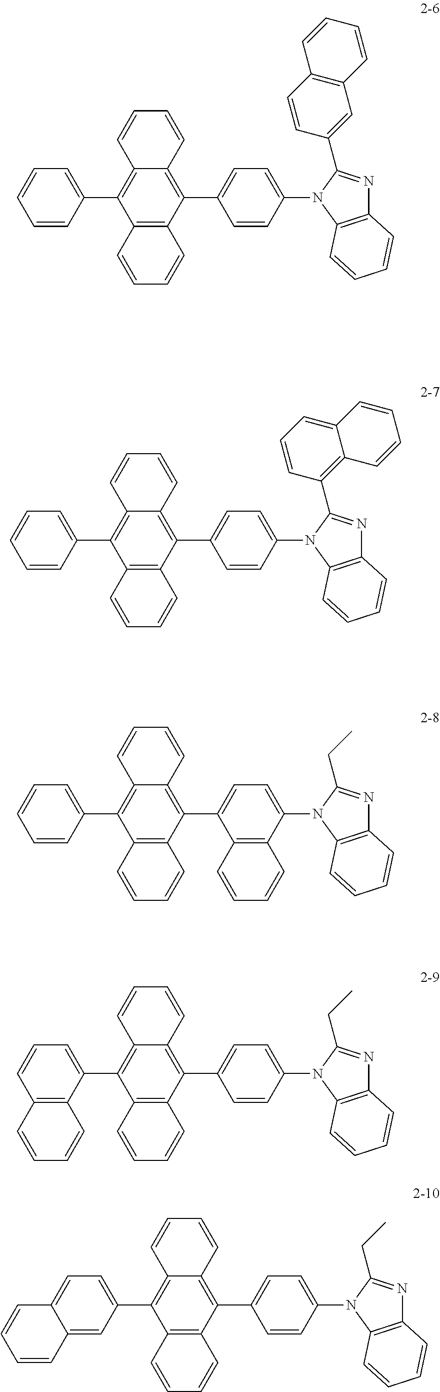 [SMILES: c1ccc(-c2c3ccccc3c(-c3ccc(-n4c(-c5ccc6ccccc6c5)nc5ccccc54)cc3)c3ccccc23)cc1 ; c1ccc(-c2c3ccccc3c(-c3ccc(-n4c(-c5cccc6ccccc56)nc5ccccc54)cc3)c3ccccc23)cc1 ; CCc1nc2ccccc2n1-c1ccc(-c2c3ccccc3c(-c3ccccc3)c3ccccc23)c2ccccc12 ; CCc1nc2ccccc2n1-c1ccc(-c2c3ccccc3c(-c3cccc4ccccc34)c3ccccc23)cc1 ; CCc1nc2ccccc2n1-c1ccc(-c2c3ccccc3c(-c3ccc4ccccc4c3)c3ccccc23)cc1]

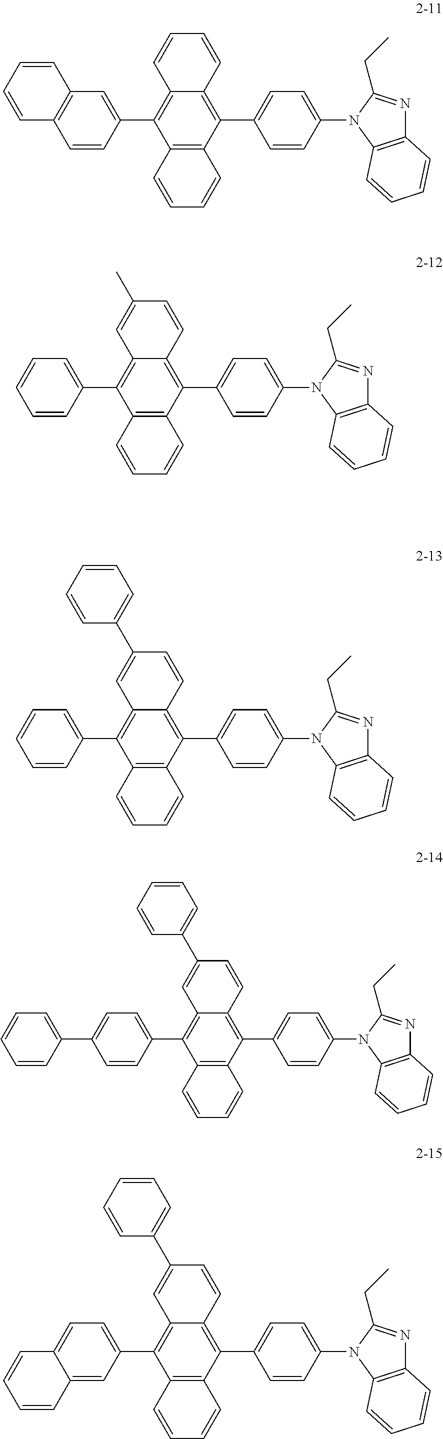 [SMILES: CCc1nc2ccccc2n1-c1ccc(-c2c3ccccc3c(-c3ccc4ccccc4c3)c3ccccc23)cc1 ; CCc1nc2ccccc2n1-c1ccc(-c2c3ccccc3c(-c3ccccc3)c3cc(C)ccc23)cc1 ; CCc1nc2ccccc2n1-c1ccc(-c2c3ccccc3c(-c3ccccc3)c3cc(-c4ccccc4)ccc23)cc1 ; CCc1nc2ccccc2n1-c1ccc(-c2c3ccccc3c(-c3ccc(-c4ccccc4)cc3)c3cc(-c4ccccc4)ccc23)cc1 ; CCc1nc2ccccc2n1-c1ccc(-c2c3ccccc3c(-c3ccc4ccccc4c3)c3cc(-c4ccccc4)ccc23)cc1]

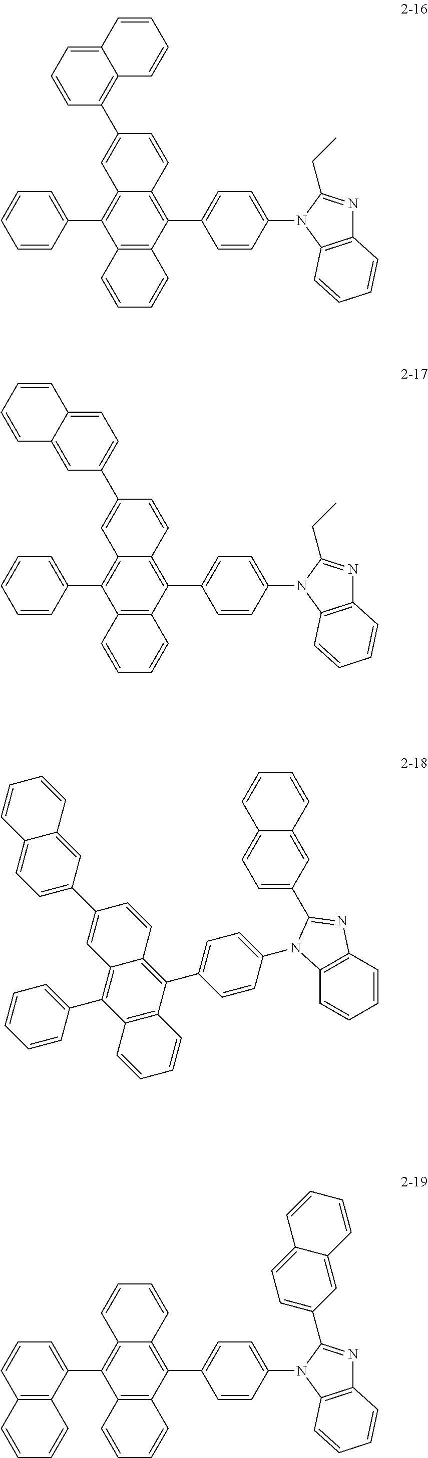 [SMILES: CCc1nc2ccccc2n1-c1ccc(-c2c3ccccc3c(-c3ccccc3)c3cc(-c4cccc5ccccc45)ccc23)cc1 ; CCc1nc2ccccc2n1-c1ccc(-c2c3ccccc3c(-c3ccccc3)c3cc(-c4ccc5ccccc5c4)ccc23)cc1 ; c1ccc(-c2c3ccccc3c(-c3ccc(-n4c(-c5ccc6ccccc6c5)nc5ccccc54)cc3)c3ccc(-c4ccc5ccccc5c4)cc23)cc1 ; c1ccc2cc(-c3nc4ccccc4n3-c3ccc(-c4c5ccccc5c(-c5cccc6ccccc56)c5ccccc45)cc3)ccc2c1]

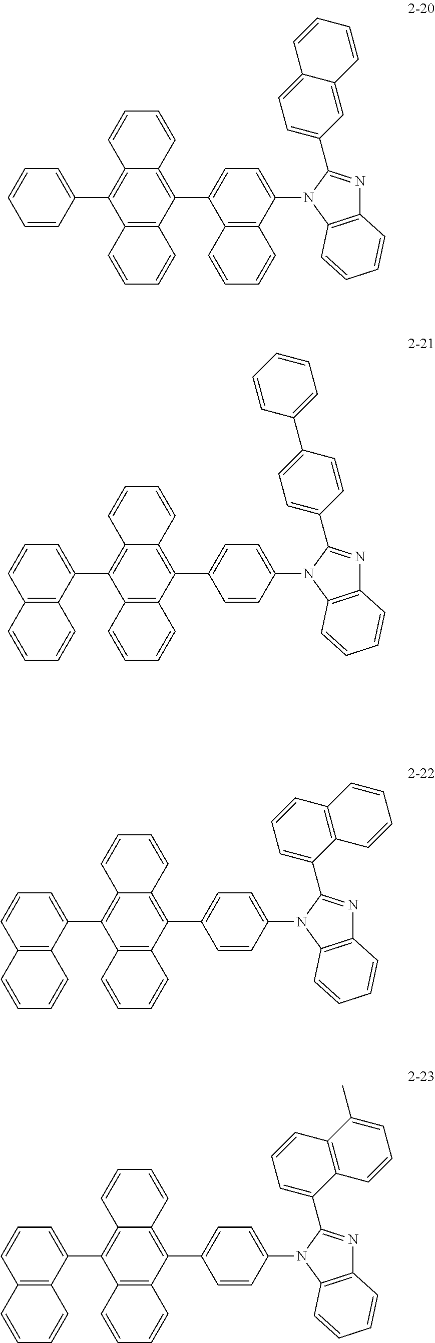 [SMILES: c1ccc(-c2c3ccccc3c(-c3ccc(-n4c(-c5ccc6ccccc6c5)nc5ccccc54)c4ccccc34)c3ccccc23)cc1 ; c1ccc(-c2ccc(-c3nc4ccccc4n3-c3ccc(-c4c5ccccc5c(-c5cccc6ccccc56)c5ccccc45)cc3)cc2)cc1 ; c1ccc2c(-c3c4ccccc4c(-c4ccc(-n5c(-c6cccc7ccccc67)nc6ccccc65)cc4)c4ccccc34)cccc2c1 ; Cc1cccc2c(-c3nc4ccccc4n3-c3ccc(-c4c5ccccc5c(-c5cccc6ccccc56)c5ccccc45)cc3)cccc12]

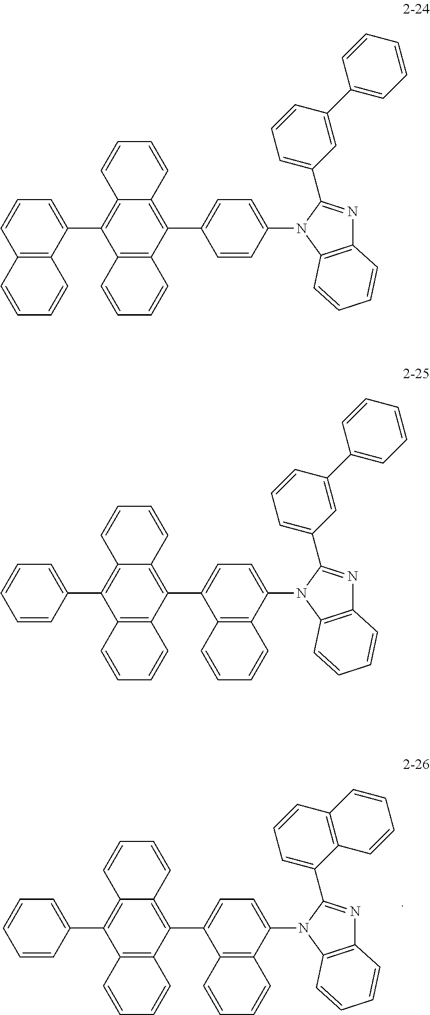 [SMILES: c1ccc(-c2cccc(-c3nc4ccccc4n3-c3ccc(-c4c5ccccc5c(-c5cccc6ccccc56)c5ccccc45)cc3)c2)cc1 ; c1ccc(-c2cccc(-c3nc4ccccc4n3-c3ccc(-c4c5ccccc5c(-c5ccccc5)c5ccccc45)c4ccccc34)c2)cc1 ; c1ccc(-c2c3ccccc3c(-c3ccc(-n4c(-c5cccc6ccccc56)nc5ccccc54)c4ccccc34)c3ccccc23)cc1]

17. The organic light emitting diode of claim 12, wherein the emissive layer has a single emitting part.

18. The organic light emitting diode of claim 12, wherein the emissive layer includes:
a first emitting part disposed between the first electrode and the second electrode;
a second emitting part disposed between the first emitting part and the second electrode;
a third emitting part disposed between the second emitting part and the second electrode;
a first charge generation layer disposed between the first emitting part and the second emitting part; and
a second charge generation layer disposed between the second emitting part and the third emitting part, and
wherein the third emitting part includes the blue emitting material layer and the electron transport layer.

19. The organic light emitting diode of claim 18, wherein the second emitting part includes at least one emitting material layer, and wherein the at least one emitting material layer includes:
a first layer disposed between the first charge generation layer and the second charge generation layer; and
a second layer disposed between the first layer and the second charge generation layer.

20. The organic light emitting diode of claim 19, wherein one of the first layer and the second layer is a red emitting material layer and the other of the first and second layers is a green emitting material layer.

21. The organic light emitting diode of claim 20, wherein the at least one emitting material layer further includes a third layer of a yellow-green emitting material layer disposed between the first layer and the second layer.

22. An organic light emitting device including:
a substrate; and
the organic light emitting diode of claim 1 disposed over the substrate.

23. An organic light emitting device including:
a substrate; and
the organic light emitting diode of claim 12 disposed over the substrate.
